(19) United States
(12) Patent Application Publication
(10) Pub. No.: US 2007/0053934 A1
(43) Pub. Date: Mar. 8, 2007

(54) Polyvalent multimeric composition containing active polypeptides, pharmaceutical compositions and methods of using the same

(76) Inventors: Neville Kallenbach, Philadelphia, PA (US); Daniel Malamud, New York, NY (US); Zhigang Liu, New York, NY (US); William Abrams, Merion Station, PA (US); Paramjit Arora, New York, NY (US)

Correspondence Address: KLAUBER & JACKSON, 411 HACKENSACK AVENUE, HACKENSACK, NJ, 07601, US

(21) Appl. No.: 11/510,493

(22) Filed: Aug. 25, 2006

Related U.S. Application Data
(60) Provisional application No. 60/711,794, filed on Aug. 26, 2005.
(60) Provisional application No. 60/788,514, filed on Mar. 31, 2006.

(51) Int. Cl. A61K 31/70; A61K 39/12; A01N 43/04

(52) U.S. Cl. 424/204.1; 514/44

(57) ABSTRACT

The invention is directed to compositions comprising polyvalent complexes containing a biocompatible polymer backbone to which is attached a plurality of monomeric anti-microbial peptides and to methods for using such complexes to stabilize anti-microbial peptides for treating or preventing a disease or condition resulting from infection with a microbe. Multivalent derivatives of existing antimicrobial peptides in which several peptides are covalently linked have been investigated. The resulting construct may contain up to 30 or more units, and exhibits a significant enhancement of anti-microbial effect relative to the free peptides: on the order of a ten fold improvement in effectiveness, suggesting that higher multimerization can indeed lead to more effective agents. The invention is also directed to the use of such complexes for delivery of anti-viral peptides, in particular, peptides and peptide fragments obtained from salivary agglutinin protein (gp-340) for use in treating or preventing infection with HIV.

### CROSS-REFERENCE TO RELATED APPLICATIONS

This application is a non-provisional application, which claims priority to provisional application Ser. No. 60/711,794, filed Aug. 26, 2005 and to provisional application Ser. No. 60/788,514, filed Mar. 31, 2006, both of which are incorporated herein by reference in their entireties. Applicants claim the benefits of these applications under 35 U.S.C. § 119(e).

### GOVERNMENT RIGHTS CLAUSE

The research leading to the present invention was supported by National Institutes of Health Grant No. DE 14825. Accordingly, the Government has certain rights in the invention.

### FIELD OF THE INVENTION

This invention relates to novel compositions containing active polypeptides, and particularly, such peptides as demonstrate antimicrobial, antifungal or antiviral activity. The invention also relates to methods for the preparation of the compositions, and their use in preventing and/or treating conditions resulting from the unwanted presence of microbial, fungal or viral activity.

### BACKGROUND OF THE INVENTION

For the last few decades it has been known that a wide range of antimicrobial peptides are secreted by all manner of multicellular organisms in response to infection by foreign viruses, bacteria or fungi. Current research focuses on the mechanism by which the peptides kill, and synthetic design strategies which can enhance the activity of the peptides to a useful therapeutic level.

A wide range of antimicrobial peptides is secreted in plants and animals to challenge attack by foreign viruses, bacteria or fungi (Boman, H. G. Antibacterial ueutides: basic facts and emerging concepts, J. htern. Med. 2003, 254 (3), 197-215). These form part of the innate immune in response to infection, which is short term and fast acting relative to humoral immunity (Medzhitov, 2000). These cationic antimicrobial peptides have been considered as prospective antibiotics agents because their effect is rapid, broad spectrum and indifferent to resistance to standard antibiotics such as penicillins (Fischetti, V. A. Novel method to control pathogenic bacteria on human mucous membranes. Ann. N.Y. Acad. Sci. 2003, 987, 207-214; Hancock, R. E. Host defence (cationic) peptides: what is their future clinical potential? Drugs 1999, 57 (4), 469-473). However, their success thus far has been limited, and is believed to be due to the requirement that they be present in a fairly high concentration to achieve killing (Hancock, 2000, PNAS), which is believed to exert a potentially cytotoxic effect on human erythrocytes as well as other cells and tissues. For these reasons current applications of these peptides are mostly topical.

Hundreds of such antimicrobial peptides have been studied extensively in order to understand the relationship between the structural features of the peptides and their antimicrobial activity, for the purpose of designing a new generation of antibiotics. Such known antimicrobial peptides are listed at (http://aps.unmc.edu/AP/database/antiV.php) and the content and disclosure of this site is incorporated herein by reference in its entirety. Representative peptides listed at the site are set forth hereinbelow by way of illustration and not limitation. Known antimicrobial peptides differ strikingly in size, sequence and structure, sharing only amphipathicity and positive charge (Hancock, R. E. Host defence (cationic) peptides: what is their future clinical potential? Drugs 1999, 57 (4), 469473; Zasloff, M. Antimicrobial peptides of multicellular organisms. Nature 2002, 415 (6870), 389-395). While the external cell wall may be the initial target, several lines of evidence suggest that antimicrobial peptides act by lysing bacterial membranes. Cells become permeable following exposure to peptides, and their membrane potential is correspondingly reduced. While the actual target and mode of action of antimicrobial peptides are incompletely understood, proposed models emphasize the need to coat or cover a significant part of the membrane in order to produce a lethal effect. In “barrel-stave” models, several peptide monomers need to bind before formation of an aggregate that inserts itself into the bilayer to form a transmembrane pore. (Ehrenstein, G.; Lecar, H. Electrically gated ionic channels in lipid bilayers. Q. Rev. Biophys. 1977, 10 (I), 1-34). In a somewhat different view, known as the “carpet model,” peptide monomers must coat the target membrane surface extensively before sections of the membrane split off as vesicles, thereby destroying the integrity of the membrane (Shai. Y.; Oren, Z. From “carpet” mechanism to de-novo designed diastereomeric cell-selective antimicrobial peptides. Peptides 2001, 22 (10), 1629-1641). Both mechanisms account for the observed threshold concentration required for peptides to achieve lethality differently. In many cases this threshold is close to that for inflicting damage on host cells or tissues, as detected by hemolysis assays for example. Thus peptides have not found wide applications except as topical agents.

Several strategies have been pursued in efforts to increase the effectiveness of antimicrobial peptides (Tam, J. P.; Lu, Y. A,; Yang, J. L. Antimicrobial dendrimeric peptides. Eur. J. Biochem. 2002, 269 (3), 923-932; Janiszewska, J.; Swieton, J.; Lipkowski, A. W.; Urbanczyk-Lipkowska, Z. Low molecular mass peptide dendrimers that express antimicrobial properties. Bioorg. Med. Chem. Lett. 2003, 13 (21). 3711-3713; Tam 2000; Dathe 2004; Tang, 1999; Dempsey, 2003; Epand, 2004; Papo, 2004). Sequence changes in natural peptides can notably reduce hemolysis while preserving activity (Staubitz, P., Peschel, A, Nieuwenhuizen, W. F., Otto, M., Gotz, F., Jung, G. and Jack, R W. (2001). Structure-function relationships in the tryptophan-rich, antimicrobial peptide indolicidin. J Pept Sci 7(10): 552-564). Inserting unnatural D-amino acids or beta-amino acids into peptide sequences, combinatorial designs based on linear or cyclic sequences (Ghadiri et al., Nature (1994), May 26; 369(6478):301-304), synthetic chemical mimetics (DeGrado et al. Protein Science, (2003)<April; 12(4):647-665; Tew et al., PNAS, (2002), April 16; 99(8):5110-5114), and multivalent dendrimeric constructs of short peptides (Janiszewska, J.; Swieton, J.; Lipkowski, A. W.; Urbanczyk-Lipkowska, Z. Low molecular mass peptide dendrimers that express antimicrobial properties. Bioorg. Med. Chem. Lett. 2003, 13 (21). 3711-3713; Xing, 2003) are other alternatives. In some cases improved solubility, salt resistance, stability and toxicity have been reported, with some reduction in IC₅₀ (Tam, et al. Antimicrobial dendrimeric peptides, Eur. J. Biochem. 2002, 269 (3), 923-932).

Accordingly, many different designs for therapeutics have been reported, seeking to develop or improve activity under physiological conditions, low toxicity and proteolytic stability. Among promising approaches, polyvalent or multivalent antimicrobial polymers offers promise for enhancing the efficacy of existing antimicrobial monomer peptide and minimizing the problems accompanying conventional antimicrobial peptides by reducing the toxicity of the residue, increasing their efficiency and selectivity, and prolonging the lifetime of the effect. Especially, these include their ability to amplify cationic charges and hydrophobic clusters as the number of monomer increases. (Tam, et al. Antimicrobial dendrimeric peptides, Eur. J. Biochem. 2002, 269 (3), 923-932). For example, the multivalency of peptides incorporated with fragments of known antibacterial peptides in dendrimers has appeared to demonstrate good activity in the design of membranolytic peptides for therapeutic applications (Tam, J. P.; Lu, Y. A,; Yang, J. L. Antimicrobial dendrimeric peptides. Eur. J. Biochem. 2002, 269 (3), 923-932).

In this connection, U.S. Pat. No. 5,229,490 to Tam discloses a particular polymeric construction formed by the binding of multiple antigens to a dendritic core or backbone, the objective of which is to potentiate the concentration of antigen within a more economical and efficient molecule. While this construction has demonstrated advantages, greater activity and corresponding stability of the construct is still an important objective that is not fulfilled therein.

U.S. Pat. No. 3,679,653 to Schuck et al. discloses the preparation and use of polymer-based protein complexes, and particularly, relates to the preparation of such complexes with hormones such as bovine growth hormone, insulin and the like. Schuck et al. however, prepare complexes with full length native hormones, and bind the native material to the polymer backbone for the purpose of improving the delivery and availability of such hormones. The inventors qualify that the level of activity of the resulting complexes are somewhat uncertain, and in any event, do not represent that any dramatic improvements in such activity are either anticipated or realized.

Moreover, the need for and appropriateness of developing an anti-HIV microbicide in 2006 are readily apparent. The HIV pandemic continues to spread, increasingly among women and children. A vaccine, perhaps the “ideal” solution, remains elusive. In the past 20 years over 50 compounds have been considered as potential microbicides, beginning with Nonoxynol-9 (N-9). More recently investigators have explored a series of relatively simple compounds (e.g., carageenan, dextrin or cellulose sulfate, and cellulose acetate 1,2-benzenedicarboxylate (Ratterree et al., 2005), “traditional” anti-retrovirals (AZT-derived NRTIs, NNRTIs, CCR5 inhibitors) and several delivery systems (e.g., bacteria, dendrimers, vaginal rings). Overall, these investigations have provided hope and guidance, but it is clear that additional approaches are still warranted. Possibilities include new drug entities, combination microbicides, new molecular targets, as well as, novel delivery systems.

The innate immune system is an ideal place to search for novel microbicidal agents. This system is designed to function at mucosal surfaces to defend against invading pathogens, and since the molecules in this system already exist at these sites, toxicity is likely to be low.

The initial studies presented here serve as a basis for further discovery and development of novel microbicidal agents, in particular anti-HIV agents. Perhaps the ultimate failure of N-9 could have been predicted, but one can still learn from the N-9 clinical trials in terms of formulation, recruitment of patients, frequency of drug application, compliance issues, and the importance of utilizing sensitive assays for cytotoxicity such as increased release of pro-inflammatory cytokines (Fichorova, R. N., Bajpai, M., Chandra, N., Hsiu, J. G., Spangler, M., Ratnam, V. and Doncel, G. F. (2004). Interleukin (IL)-1, IL-6, and IL-8 predict mucosal toxicity of vaginal microbicidal contraceptives. BioI Reprod 71(3): 761-769). Potential microbicide candidates require extensive studies to understand their mechanisms of action as well as to ascertain that the molecules are safe to mucosal surfaces and that they do not disrupt endogenous protective microflora (Patton, D. L., Sweeney, Y. C., Rabe, L. K. and Hillier, S. L. (1996). The vaginal microflora of pig-tailed macaques and the effects of chlorhexidine and benzalkonium on this ecosystem. Sex Transm Dis 23(6): 489-493; Clarke, J. G., Peipert, J. F., Hillier, S. L., Heber, W., Boardman, L., Moench, T. R. and Mayer, K. (2002). Microflora changes with the use of a vaginal microbicide. Sex Transm Dis 29(5): 288-293; Zaneveld, L. J., Waller, D. P., Anderson, R A, Chany, C., 2nd, Rencher, W. F., Feathergill, K., Diao, X. H., Doncel, G. F., Herold, B. and Cooper, M. (2002). Efficacy and safety of a new vaginal contraceptive antimicrobial formulation containing high molecular weight poly(sodium 4-styrenesulfonate). BioI Reprod 66(4): 886-894).

In addition, for a microbicide to be successful it must be affordable. Early in any research program designed to develop a microbicide, detailed consideration must be given to ways to produce the material at a cost that assures availability in the regions of the world where the epidemic is most pronounced and where these drugs are needed most. It is with respect to the development of cost effective anti-HIV agents with low toxicity that the present invention is directed.

From the above, it remains that a continuing need exists for the development of modalities that can deliver effective antibiotic peptides in a manner that confers both improved stability and economy of the therapeutic, but importantly, significantly improves the therapeutic efficacy and strength of the resultant molecule. It is toward the fulfillment of these and other related objectives that the present invention is directed.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention.

### SUMMARY OF THE INVENTION

It has now been found that a complex of a biocompatible polymer backbone and a plurality of monomeric antibiotic peptide molecules covalently bound thereto, may be prepared, that provides enhanced stability, such as resistance to enzymatic digestion, along with dramatically increased activity of the antibiotic active. In this latter regard, increases in activity on the order of ten-fold or more, over the same peptides in conventional form, are attained. This finding leads to novel compounds having therapeutic value. It also leads to pharmaceutical compositions having the polymer-peptide complex of the present invention as an active ingredient and to their use to treat, prevent or ameliorate a range of conditions in mammals of various genesis or etiology, however, primarily caused by bacteria, viruses, or fungi.

Accordingly, in a first aspect of the invention, a polyvalent polymeric product is disclosed which comprises a polymer backbone to which are covalently bonded a plurality of peptide monomers having activity against agents selected from bacteria, viruses and fungi, to generate a polyvalent effect against such bacteria, viruses or fungi. The peptide component of the complex may include any sequence of amino acids that has a lethal effect on bacteria, viruses or fungi in its monomeric form. More particularly, the peptides may be any antimicrobial peptides, including natural products found in organisms, fragments of natural peptides, and any synthetic analogs or de novo designs. These peptides can accordingly include nonnatural amino acids: beta-amino acids, d-amino acids and/or non-indigenous amino acids.

In a particular aspect the peptide of the invention may be selected from Antiviral protein Y3, Alloferon 1, Lactoferricin B, hexapeptide, Tricyclic peptide RP, indolicidin, GNCP-1, GNCP-2, HNP-1 Defensin, HNP-2 Defensin, HNP-3 Defensin, CORTICOSTATIN III (MCP-1), CORTICOSTATIN IV (MCP-2), NP-3A defensin, Protegrin 2, Protegrin 3, Protegrin 4, Protegrin 5, RatNP-1, RatNP-2, RatNP-3, RatNP-4, Caerin 1.1, Circulin A (CIRA), Circulin B (CIRB), Cyclopsychotride A (CPT), Ginkobilobin, Alpha-basrubrin. The forgoing representative listing is taken from (http://aps.unmc.edu/AP/database/antiV.php) and the content and disclosure of this site is incorporated herein by reference in its entirety. Representative peptides listed at the site are set forth herein by way of illustration and not limitation, and the present invention is considered to include the use of all such peptides within its scope.

In a more particular embodiment, the antimicrobial peptide may be selected from those containing at least one combination of one or more recurring units of arginine (R) and tryptophan (W), such as those described in the examples, and in any orientation with respect to each other. The RW peptides may be protected or unprotected, and if protected, may be protected, for example, by an amide group. The antimicrobial peptide may be linear or branched. The antimicrobial peptide may range in size from about 2 to more than about 50 amino acids. More particularly, the antimicrobial peptide may range in size from about 10 to about 25 amino acids, and more particularly, from about 4 to about 16 amino acids. In another particular embodiment, the antimicrobial peptide may comprise salivary agglutinin protein, gp340, and any one or more active fragments, mimics, analogs or derivatives thereof, or a combination of any of the foregoing. In another particular embodiment, the antimicrobial peptide is an antiviral peptide, which consists essentially of a segment of SAG (gp340) that binds to the gp120 envelope of a retrovirus, such as HIV. In a more particular embodiment, the antimicrobial peptide is an antiviral peptide derived from SAG (gp340), and more particularly, a peptide selected from any one of SEQ ID NOS: 1, 2, 3, 4 and 7, or a combination thereof.

Suitable polymers may include linear polymers or copolymers such as those described in greater detail hereinafter, or nonlinear structures, such as dendrimers. Any molecule with multiple reactive sites can be a suitable scaffold for the peptide monomers that are a part of the complexes of the invention. In a particular embodiment, the polymers for use in the preparation of the polymeric backbone may comprise a polymer (a) comprising chains of repeating caxboxylic acid or carboxylic acid anhydride units, or (b) comprising units of carboxylic acid or carboxylic acid anhydride groups separated by carbon chains of at least one and not more than four carbon atoms, said carbon chains being part of a unit which contains a maximum of eighteen carbon atoms, said polymer being formed by polymerization of polymerizable acids or anhydrides or by copolymerizing a polymerizable acid or anhydride with another copolymerizable monomer, and preferably wherein the starting acid or anhydride and any additional copolymerizable monomer are unsaturated and such polymerization or copolymerization comprises addition type polymerization or copolymerization involving such unsaturation. As indicated, particular polymers include maleic anhydride homo- and copolymers, such as polymaleic anhydride (PMA) and ethylene/maleic anhydride copolymers (PEMA).

The present invention provides novel products in which a plurality of monomeric antimicrobial/antiviral/antifungal peptides are covalently bonded to a selected polymer and a process for their production. These novel polymer-peptide products have valuable diagnostic and therapeutic properties.

Unexpectedly it has been found that the polymer-peptide complexes of the invention can be prepared which not only offer increased stability relative to the individual antimicrobial peptide, but demonstrate many orders of magnitude enhancement of activity over the peptide alone.

In a further embodiment of the invention, the method for the preparation of the composition or complex comprises covalently grafting or attaching monomeric peptides on a polymer scaffold. More particularly, the method of preparation comprises the reaction of the monomeric peptide material with the polymer backbone, as by diazo coupling, to form the resultant polymer complex. The number of monomer peptides on polymer scaffold depends on the number of reactive sites on each polymer.

In a further aspect, the polymer-peptide complexes of the invention may be used to treat microbial or fungal conditions affecting lower animals, and possibly, plants. The complexes could be designed and assembled to include the peptides pertinent for the treatment of a particular microbe or fungus of interest, and then formulated into appropriate compositions and dosage forms for administration or application to an affected host.

In a further aspect, the present invention provides pharmaceutical compositions comprising a polymer-peptide complex of the invention, and a pharmaceutical carrier, excipient or diluent. In this aspect of the invention, the pharmaceutical composition can comprise one or more variant complexes, prepared, for example, with a differing array of peptide monomers, to afford a more comprehensive treatment in the instance where a multiplicity of microbial/viral/fungal antigens are known to be present.

In a further aspect of the invention, a method is disclosed for treating mammals, including humans, as well as lower mammalian species, susceptible to or afflicted with a condition attributable to or resulting from a bacterial, viral or fungal infection, which method comprises administering an effective amount of a pharmaceutical composition containing or comprising the polymer-peptide complex just described.

In additional aspects, this invention provides methods for synthesizing the complexes of the invention, with representative synthetic protocols and pathways disclosed later on herein.

In another aspect of the invention, this invention relates to the identification of fragments of the salivary agglutinin protein, gp340, that exhibit anti-retroviral activity. More particularly, a peptide comprising the amino acid sequence of SEQ ID NO: 1 has been shown to inhibit the infectivity of HIV and thus prevent its spread. This activity is due to its ability to bind to the gp120 envelope of the HIV virus, more particularly to the stem of the V3 loop of the envelope protein. Two smaller peptides, an 11 mer and a 16 mer, obtained from this larger peptide, have been identified as having the capability to bind to the same site on the stem of the V3 loop of the HIV gp120 envelope protein. These two peptides have the amino acid sequences as set forth in SEQ ID NOS: 2 and 3. The present invention also provides methods of stabilizing the active anti-HIV peptide fragments through particular chemical modifications or by attaching the peptides or analogs thereof to a biocompatible polymeric scaffold or presenting the peptides or fragments by way of a dendrimeric complex. Such modifications may provide for prevention of enzymatic breakdown of the peptides and thus aid in enhancing the anti-viral activity by prolonging the half-life of the proteins. The fragments or analogs or mimics thereof may be formulated in a pharmaceutical compositions for delivery via the oral or parenteral routes. They may be formulated for topical delivery, more particularly, for delivery to a mucosal surface, including the oral mucosa, the vaginal mucosa or the rectal mucosa. The soluble gp340 peptide fragments and pharmaceutical compositions comprising these fragments or mimics thereof may be administered prophylactically or therapeutically to a person suffering from an HIV infection or to a person at risk for acquiring an HIV infection. Since the peptides obtained from gp340 bind to the gp120 envelope of HIV, it is also contemplated that these peptides or derivatives, fragments or mimics thereof may be used to purify blood products to eliminate any possible contamination of the blood product with a retrovirus prior to use of the blood product for transfusions. Alternatively, the peptides, or fragments or derivatives or mimics thereof may be used in plasmapheresis procedures to clear the blood of patients having or suspected of having a retroviral infection.

Another aspect of the invention provides a method of inhibiting the infectivity of a retrovirus by contacting a retrovirus virion with a substantially purified preparation of a soluble fragment of gp340, or an analog, or mimic thereof and incubating the virion with the soluble fragment of gp340 or analog or mimic thereof for a period of time sufficient to inhibit the infectivity of the retrovirus.

Another aspect of the invention provides a method of inhibiting or preventing gp 120-mediated binding or spread of a retrovirus to a host cell, comprising contacting a retrovirus virion with a composition comprising a soluble gp340 fragment, or analog, or mimic thereof for a period of time sufficient to inhibit the spread of the retrovirus to the host cell.

Another aspect of the invention provides a method of inhibiting infectivity of a retrovirus, comprising treating a subject with a pharmaceutical composition comprising a therapeutically effective amount of a soluble gp340 fragment, or analog, or mimic thereof, and a pharmaceutically acceptable carrier.

In one particular embodiment, the soluble fragment consists essentially of a segment of gp340 that binds to the gp120 envelope glycoprotein of a retrovirus. In a more particular embodiment, the fragment consists essentially of the N-terminal sequence of gp340, as set forth in SEQ ID NO: 1. In another particular embodiment, the fragment consists essentially of a consensus scavenger receptor cysteine rich (SRCR) sequence consisting essentially of the amino acid sequence as set forth in SEQ ID NO: 4. In a more particular embodiment, the fragment consists essentially of the amino acid sequences as set forth in SEQ ID NOS: 2, 3 or 7. In yet another particular embodiment, the soluble gp340 fragment consisting essentially of the sequence of any one of SEQ ID NOs: 1, 2, 3, 4 or 7 may contain one or more conservative amino acid substitutions. In yet another particular embodiment, the soluble peptide fragment of the gp340 protein is administered topically. In yet another particular embodiment, the soluble peptide fragment of the gp340 protein is administered to a mucosal surface. In a more particular embodiment, the mucosal surface is selected from the oral mucosa, the rectal mucosa, and the vaginal mucosa. In yet another particular embodiment, the soluble gp340 fragment, analog or mimic thereof may be administered prophylactically or therapeutically. In yet another particular embodiment, the soluble peptide fragment of the gp340 protein possesses virucidal activity. In yet another particular embodiment, the soluble gp340 fragment, analog or mimic thereof is effective against multiple clades of human immunodeficiency virus or against multi-drug resistant strains of human immunodeficiency virus.

In yet another particular embodiment, the retrovirus is a human immunodeficiency virus. In a more particular embodiment, the human immunodeficiency virus is HIV-1.

Another aspect of the invention provides for methods for stabilizing the soluble gp340 fragments, or analogs or mimics thereof.

In one embodiment, the gp340 fragment, analog or mimic thereof is stabilized by covalent modification of the peptide structure to incorporate one or more disulfide or olefinic H-bonds into the peptide structure. In another particular embodiment, the soluble gp340 fragment, analog or mimic thereof is stabilized by attachment to a biocompatible polymeric scaffold or presented in a polyvalent dendrimeric complex. In another particular embodiment, the biocompatible polymeric scaffold comprises a polymaleic anhydride and poly(ethylene/maleic anhydride) copolymer, wherein the copolymer has a molecular weight in the range of about 1,000 to about 1,000,000. In another particular embodiment, the polyvalent dendrimeric complex comprises a biocompatible polymer selected from the group consisting of three or more lysine residues and an optional spacer molecule, polyamidoamine, and a plurality of monomeric peptides covalently bound thereto, wherein the peptides are selected from the group consisting of a soluble gp340 fragment, analog or mimic thereof as set forth in SEQ ID NOS: 1, 2, 3, 4, or 7 and wherein the polyvalent dendrimeric complex exhibits activity that ranges up to on the order of at least 2 times the activity demonstrated by the peptides alone, more preferably about 5 to 10 times the activity demonstrated by the peptides alone. In yet another particular embodiment, the covalent modification results in stabilization of the peptide structure without the need for introducing side claims that alter the affinity of the peptide for the target.

Another aspect of the invention provides a conformationally restricted protein-secondary structure mimetic of a soluble gp340 fragment that exhibits anti-HIV activity.

In one embodiment, the mimetic exhibits anti-HIV activity that ranges up to or on the order of about 2 to 10 times the activity of the parental anti-HIV peptide, and wherein the parental anti-HIV peptide consists essentially of the sequence of any one of SEQ ID NOs: 1, 2, 3, 4 or 7. The mimetic is identified on the basis of its ability to bind to the stem of the V3 loop of the HIV envelope protein and prevent the infectivity or spread of the HIV virus.

Another aspect of the invention provides a pharmaceutical composition comprising a peptide or peptide fragment of soluble gp340, or an analog or mimic thereof and a pharmaceutically acceptable carrier. The composition may comprise the gp340 peptide fragment in its original form, or it may contain a stabilized form of the soluble gp340 peptide fragment or the gp340 peptide fragment attached to a biocompatible scaffold or presented in a dendrimeric complex.

In one particular embodiment, the composition comprising the peptide or peptide fragment, analog or mimic thereof consists essentially of the gp120 binding domain of gp340. The gp120 binding domain of gp340 consists essentially of an N-terminal sequence of gp340 as set forth in SEQ ID NO: 1 or a peptide of about 11 to 16 amino acids in length as set forth in the amino acid sequence of any one of SEQ ID NOs: 2, 3 or 7. The peptide or peptide fragment, analog or mimic thereof inhibits infectivity of a retrovirus, more particularly, human immunodeficiency virus, most particularly, HIV-1. The composition comprising one of the amino acid sequences of SEQ ID NOs: 1, 2, 3, 4 or 7 may contain one or more conservative amino acid substitutions. The pharmaceutical composition containing the gp340 fragment, analog or mimic thereof exhibits virucidal activity. The pharmaceutical composition comprising the soluble gp340 fragment, analog or mimic thereof may be effective against multiple clades of human immunodeficiency virus or against multi-drug resistant strains of human immunodeficiency virus.

In yet another particular embodiment, the pharmaceutical composition is formulated for topical delivery. In yet another particular embodiment, the pharmaceutical composition is formulated for delivery to a mucosal surface. In yet another particular embodiment, the mucosal surface is selected from the group consisting of the oral mucosa, the rectal mucosa, and the vaginal mucosa.

In yet another particular embodiment, the gp340 fragment, analog or mimic thereof within the pharmaceutical composition, may be stabilized by covalent modification of the peptide structure to incorporate one or more disulfide or olefinic H-bonds into the peptide structure. Alternatively, in yet another particular embodiment, the soluble gp340 fragment, or analog, or mimic thereof within the pharmaceutical composition is attached to a biocompatible polymeric scaffold or presented in a polyvalent dendrimeric complex. In yet another particular embodiment, the biocompatible polymeric scaffold comprises a polymaleic anhydride and poly(ethylene/maleic anhydride) copolymer, and the copolymer has a molecular weight in the range of about 1,000 to about 1,000,000. The polyvalent dendrimeric complex comprises a biocompatible polymer selected from the group consisting of three or more lysine residues and an optional spacer molecule, polyamidoamine, and a plurality of monomeric peptides covalently bound thereto. The peptides are selected from the group consisting of a soluble gp340 fragment, analog or mimic thereof as set forth in SEQ ID NOS: 1, 2, 3, 4 and 7, and the polyvalent dendrimeric complex exhibits activity that ranges up to on the order of 2 to 10 times the activity demonstrated by the peptides alone. In yet another particular embodiment, the pharmaceutical composition comprises a conformationally restricted protein-secondary structure mimetic having anti-HIV activity, wherein the mimetic exhibits anti-HIV activity that ranges up to or on the order of 2 to 10 times the activity of the parental anti-HIV peptide, wherein the parental anti-HIV peptide consists essentially of the sequence of any one of SEQ ID NOs: 1, 2, 3, 4, or 7 and wherein the mimetic is identified on the basis of its ability to bind to the stem of the V3 loop of the HIV envelope protein (SEQ ID NOS: 6 or 8) and prevent the infectivity or spread of the HIV virus.

Another aspect of the invention provides a means for stabilizing the gp340 fragment or mimic thereof. In one particular embodiment, covalent modifications of sequences that have been identified to stabilize secondary structure and affinity via disulfide and H-bond surrogate chemistries will be employed. In another particular embodiment, the gp340 peptides have the amino acid sequences as set forth in SEQ ID NOS: 1, 2, 3, 4 or 7. In yet another particular embodiment, multivalent complexes of peptides and analogs are prepared to enhance avidity for the gp120 target by multivalency and increase the half-life of activity. In yet another particular embodiment, a pentameric structure is proposed to provide a rigid display of molecules having anti-HIV activity.

In another particular embodiment, one means of enhancing the stability of the soluble gp340 fragments or analogs or mimics thereof is to provide a polyvalent complex of a biocompatible polymer, wherein the biocompatible polymer is selected from a polymaleic anhydride and poly (ethylene/maleic anhydride) copolymer, and wherein the biocompatible polymer has a molecular weight in the range of about 1,000 to about 1,000,000; and a plurality of monomeric peptides that inhibit HIV infectivity covalently bound thereto. The peptides are selected from the group consisting of SEQ ID NOs: 1, 2, 3, 4, or 7 or fragments, analogs or mimics thereof, and the polyvalent complex exhibits activity that ranges up to on the order of 10 times the activity demonstrated by the peptides alone.

In yet another particular embodiment, another means of enhancing the stability of the soluble gp340 fragments or analogs or mimics thereof is to provide a polyvalent dendrimeric complex, wherein the complex comprising a biocompatible polymer is selected from the group consisting of three or more lysine residues and an optional spacer molecule, or polyamidoamine, and a plurality of monomeric peptides covalently bound thereto. The peptides are selected from the group consisting of soluble gp340 fragments, analogs or mimics thereof as set forth in SEQ ID NOS: 1, 2, 3 and 4, and the polyvalent dendrimeric complex exhibits activity that ranges up to on the order of about 2 to 10 times the activity demonstrated by the peptides alone.

In yet another particular embodiment, the methods provided for inhibiting HIV-1 infectivity comprise contacting the HIV-1 virion with the pharmaceutical composition containing any one of the gp340 fragments or analogs or mimics thereof in their original form or in a more stable configuration for a time period sufficient to prevent or inhibit HIV-1 infectivity. The prevention or inhibition of HIV-1 infectivity is achieved by the ability of the gp340 peptide fragments in the composition to bind to the stem of the V3 loop of the gp120 HIV-1 envelope. Moreover, the binding of the gp120 envelope of HIV-1 by the gp340 peptide fragments occurs in the stem of the V3 loop of the envelope protein.

A further aspect of the invention provides for the design of chemically modified peptides from the HIV binding region of gp340. In one embodiment the invention provides for the sequence analysis of the HIV binding interface, development of conformationally restricted analogs, synthesis of multivalent molecules from active peptide analogs, and the synthesis of dendrimeric constructs. In another embodiment, truncated and sequence variants of free peptides derived by small library (phage display) syntheses of analogs of SRCR1 to identify variants with increased binding and anti-HIV activity will be employed.

Another aspect of the invention provides for quantitative assessment of the SRCR analogs and multivalent complexes for anti-HIV-1 activity, binding activity to target peptides from gp120, potential in vitro cytotoxicity, and an evaluation matrix.

As evidenced from the studies presented herein, gp340 is an excellent candidate to serve as a model for an HIV microbicide. It is present at most mucosal surfaces, it binds to HIV-1 at a conserved sequence at the stem of the V3 loop, and it is effective against a wide range of HIV-1 strains (See U.S. Pat. No. 6,287,605). Accordingly, the invention provides for preparation and identification of a series of low molecular weight mimetics based on an identified amino acid sequence present in gp340, a glycoprotein present in the innate immune system. As noted previously, gp340 belongs to the SRCR (scavenger receptor cysteine rich) superfamily sharing a common domain of about 110 amino acids. gp340 appears to be the major inhibitor of HIV infection present in human saliva. Analysis revealed that this glycoprotein is present on mucosal surfaces throughout the body (tears, saliva, bronchial alveolar fluid, cervical/vaginal fluid). gp340 consists of 13 highly conserved SRCR domains, separated by short non-SRCR regions, a CUB domain, a ZP domain and a final SRCR domain. Inhibition of HIV-1 infection involves binding to Env at a conserved sequence in the stem of the gp120 V3 loop. Cloning of the single N-terminal SRCR identified a truncated molecule that maintained the ability to bind to the same sites on gp120 as the intact gp340 and inhibit HIV-1 infectivity. Within this single SRCR, the present invention provides for the identification of an 11 amino acid sequence which also binds to the same amino acid sequence on gp120, and which has low, but measurable anti-HIV activity. It is proposed that the low anti-viral activity is due to either a loss of 3D structure in the 11 mer, or to the requirement for multiple entities. Accordingly, in another aspect of the invention, methods are provided for generating (1) a series of mimetics using a phage display library of the 11 mer sequence, (2) chemical stabilization of the 11 mer via S—S or H-bond surrogates, and (3) multimeric constructs with linear polymers and dendrimers. The invention further provides for methods for evaluation of the mimetics for their ability to bind to gp120 and to inhibit HIV-1 infectivity. The invention further provides for methods whereby the structures with high efficacy (as determined by ED50) and low cytotoxicity (LD50, and cytokine release) and hence a high therapeutic index, will be used to generate gel formulations and measure release of active drug, stability, anti-HIV activity, and cytotoxicity in cells and ex vivo tissues. The invention further provides for methods of determining the ability of the microbicides to inhibit multiple clades, multi-drug resistant HIV-1 strains, and function in the presence of serum and cervical vaginal lavage (CVL). Candidate microbicides with the best performance will be advanced into animal toxicity studies, and ultimately into human phase 1 safety trials.

Another aspect of the invention provides for the use of the gp340 peptides, or fragments, derivatives or mimics thereof as a means of purifying blood or blood products to aid in the possible elimination of HIV contaminants in the blood or blood products. Moreover, it is envisioned that due to the capability of the gp340 peptides to bind the gp120 envelope protein of HIV, one may contemplate the use of gp340 peptides, fragments, derivatives or mimics thereof for use in plasmapheresis to help eliminate viral particles from the blood to aid in reduction of HIV viral load.

Other objects and advantages will become apparent to those skilled in the art from a review of the following description which proceeds with reference to the following illustrative drawings.

### BRIEF DESCRIPTION OF THE DRAWINGS

FIG. 1. Expression of recombinant gp-340 and inhibition of HIV-1 infection. (a) Culture supernatants of transiently transfected 293/T cells were analyzed by SDS-PAGE and Western blot and stained with polyclonal anti-serum 1527. SAG purified from saliva served as positive control and culture supernatants from 293/T cells or 293/T cells transfected with plasmid without DMBT-1 served as negative control. (b) IIIB virus was pre-treated with culture supernatant collected from 293/T cells transiently transfected with either a DMBT-1 expression construct or the vector only construct before the virus was added to indicator cells, H9. 10 nM AZT served as a control for inhibition of viral replication. After 6 days of infection, culture supernatant was collected for p24 determination. Results shown are % inhibition+/−SEM for triplicate determinations.

FIG. 2. (a). Schematic presentation of the N-SRCR construct (b) Western blot analyses of the Ni+-chelate column purified N-SRCR using an Mab specific for the gD tag (c) Coomassie Blue staining of the purified eluate.

FIG. 3. Antibody reactivity with purified N-SRCR protein. (a) Purified N-SRCR or SAG protein immobilized onto an ELISA plate was detected with antibodies specific for SAG (Ab1527 and Ab1529, Mab143) or gp-340 (Mab213-06) or HSV gD (anti-HSV). Results shown are the average +/−SEM for triplicate samples; (b) Purified N-SRCR or SAG was reduced with 50 mM DTT and analyzed on SDS-PAGE and Western blot, stained with Ab1527 polyclonal antibody raised against native SAG, Ab1529 polyclonal antibody raised against denatured SAG, or Ab-9D which detects the HSV tag on N-SRCR.

FIG. 4. N-SRCR interaction with HIV-1 gp120 and env peptides. (a) an equal molar concentration of N-SRCR or gp340 was incubated with gp120_{BaL} captured by a N terminus-specific antibody, D7324, and the binding was detected with a gp-340-specific Mab 213-06; (b, c) N-SRCR protein was allowed to bind to HIV-1 gp120_{MN} peptide (P6283) immobilized onto 96-well plate in the presence of either 4 mM Ca++ or 5 mM EDTA. The bound protein was detected with either anti-gD Mab for N-SRCR or Ab 1527 for SAG and alkaline phosphatase-conjugated secondary antibodies. (d) N-SRCR was allowed to bind captured gp 120BaL following sCD4 binding to gp120; CD41 Mab 17b binding indicated the gp120 conformational change induced by sCD4. The affinity purified pooled HIV+ human IgG (HIVIG) served as a control for the amount of a gp120 immobilized. The dotted line indicates the normalized binding of each antibody in the absence of sCD4.

FIG. 5. Purified N-SRCR or gp-340 reactivity with gp12O_{MN} peptides. N-SRCR or gp-340 was incubated with immobilized peptides and the binding was detected with gp-340-specific Mab 213-06. The OD₄₁₀ values were normalized against P6283 that was indicated by the dotted line.

FIG. 6. Differential reactivity of antibodies with N-SRCR bound to immobilized P6283. Purified N-SRCR protein was allowed to bind immobilized P6283 and detected with serial dilutions of antibodies specific for conformational epitope(s) (Ab527) or gD tag (anti-HSV). The OD410 values are the means of duplicate wells.

FIG. 7. Inhibition of HIV-1 infection by N-SRCR. 5 ng/ml virus was pre-incubated for 60 min at 37° C. with serial dilutions of purified N-SRCR protein before H9 cells (for NL4-3 and IIIB) or IL-2 stimulated human PBMCs (for BaL) were infected. The inhibitory effect of N-SRCR was determined by measuring p24 in the culture supernatant. The assay was conducted in triplicate wells and the values reflected the averages +/−SEM of the readout. Inhibition of infection was expressed as the percent of p24 reduction of experimental wells vs. the controls.

FIG. 8. Effect of gp-340 or N-SRCR on HIV-1 attachment and infection of cells. The H9 cells were treated with increasing concentrations of gp-340 (A and B) or N-SRCR (C) before and during HIV-1 exposure. After 4 hours, the cells were extensively washed of virus and either treated with or without trypsin at room temperature for 10 min and lysed for total cell-associated p24 measurement or re-fed with fresh medium to continue culture for 4 days to determine infection (data only for gp-340 shown). The values were average of triplicate wells and bars represent standard errors.

FIG. 9. Expression of recombinant gp340 and inhibition of HIV-1 infection. (A) Culture supernatants of transfected 293/T cells were analyzed by SDS-PAGE and Western blot and stained with polyclonal antibody 1527. SAG purified from saliva served as positive control and culture supernatants from 293/T cells or 293/T cells transfected with plasmid without gp340 served as negative control. (B) IIIB virus was pre-treated with culture supernatant from 293/T cells transfected with gp340 construct or vector only then added to H9 cells. AZT served as a positive control. After 6 days, culture supernatant was collected for p24 determination. Results shown are % inhibition ±SEM for triplicate determinations, and inhibition of HIV-1 infection. (A) Culture supernatants of transfected 293/T cells were analyzed by SDS-PAGE and Western blot and stained with polyclonal antibody 1527. SAG purified from saliva served as positive control and culture supernatants from 293/T cells or 293/T cells transfected with plasmid without gp340 served as negative control. (B) IIIB virus was pre-treated with culture supernatant from 293/T cells transfected with gp340 construct or vector only then added to H9 cells. AZT served as a positive control. After 6 days, culture supernatant was collected for p24 determination. Results shown are % inhibition ±SEM for triplicate determinations.

FIG. 10. Domain structure of gp340. Modified from Holmskov et al. (Holmskov, U., Mollenhauer, J., Madsen, J., Vitved, L., Gronlund, J., Tornoe, I., Kliem, A., Reid, K. B., Poustka, A. and Skjodt, K. (1999). Cloning of gp-340, a putative opsonin receptor for lung surfactant protein D. Proc Natl Acad Sci USA 96(19): 10794-10799).

FIG. 11. A. Schematic showing the expression construct for the first SRCR domain from gp340. PCR primers with restriction sites at the 5′ ends spanning the leader sequence, the first SRCR, and one half of the first SID were used on the gp340 plasmid. The resulting PCR product was digested and ligated into the pTriEx 3 expression vector containing a CMV promoter and a G418 selection marker (Novagen, San Diego, Calif.) so that the C-terminal His-6 and HSV gD sequence tags were in frame. The plasmid was sequenced and transfected into 293/T cells using the Fugene 6 reagent per manufacturer's instructions (Roche, Alameda, Calif.) selected in G418 at an early time point and high producing stable clones were generated by limiting dilution cloning and screened by SDS gel electrophoresis and Western blots, probed with a polyclonal anti-serum raised against gp340, Ab-1527, or a commercial Mab against the HSV tag, anti-gD (Wu et al., 2006). B. Inhibition of HIV-1 infection by N-SRCR. 5 ng/ml virus was pre-incubated for 60 min at 37° C. with serial dilutions of purified NSRCR protein before H9 cells (for NL4-3 and IIIB) or IL-2 stimulated human PBMCs (for BaL) were infected. The inhibitory effect of N-SRCR was determined by measuring p24 in the culture supernatant 3-4 days later. The assay was conducted in triplicate wells and the values reflected the averages ±SEM of the readout. Inhibition of infection was expressed as the percent of p24 reduction of experimental wells vs. the samples with cells and virus only ±SEM. C. Cartoon of HIV-1 gp120 secondary structure (green) with the SRCR binding domains highlighted in yellow and the primary site at the stem of the V3 loop in red (arrow).

FIG. 12. Binding of gp340, 16 mer or 11 mer peptide to S. mutans KPSK2. Note that the concentration of gp340 (nM) is 3 orders of magnitude less than that of the peptides (pM).

FIG. 13. Structural model for the bioactive domain of SRCR based on the crystal structure of M2BP, a single SRCR domain. Residues in red (VEVL •••• W) were those required for binding to streptococci, based on alanine scanning of the 16 mer.

FIG. 14. Far UV CD spectra of 11 mer and 16 mer SRCR peptides in 100 mM Na carbonate buffer, pH 9.6, 20° C. from 190-260 nm, are the average of 10 scans. The green curve represents a subtraction of the spectrum for the 11 mer from that of the 16 mer. The results are expressed in terms of molar residue CD.

FIG. 15. Observation of fluorescent quenching upon mixing gp340 16 mer with gp120 target peptide (A) or a control peptide (B).

FIG. 16. Monomers and oligomeric complexes of the antimicrobial peptide indolicidin and its retro-sequence analog R-indolicidin.

FIG. 17. Summary of the research phases of the invention. Studies were initiated with gp340, the HIV inhibitor present at mucosal surfaces, and then progressed to a truncated 35 kDa N-SRCR domain and an internal 11 mer HIV-1 binding structure. The invention provides for methods to stabilize and or multiply the SRCR derived 11 mer. Active mimetics are selected to advance into microbicide development.

FIG. 18. Outline of panning procedure to enrich a library of M13 phage fused to randomized 7 mer inserts for binding to a protein target.

FIG. 19. Three conformationally restricted candidates to be evaluated for gp120 binding. The sequence shown in Structure 1 is the 11 mer from SCRC that shows streptococcal and gp120 binding activity. Structure 2 shows a version of the same sequence immobilized by disulfides. Structure 3 is a hydrogen bond surrogate (HBS) analog of peptide 1.

FIG. 20. Synthesis of HBS 13-sheet, Structure 3.

FIG. 21. Structure of tetravalent peptide with and without spacers. Structures of peptide backbone and alternative spacer molecules (3-alanine and polyethylene glycol (PEG), where the peptide(s) are derived from SRCR active sequences.

FIG. 22. Synthetic scheme for constructing dendrimeric antiviral peptides based on free or constrained peptide sequences and a GO-PAMAM. PAMAM generations GO, G1, G2 have 4, 8 and 16 amino groups, respectively.

FIG. 23. Hemolytic index (HI) values. HI defined as ratio of HD₅₀ to IC₅₀ indicates the selectivity of (RW)ₙ—NH₂ peptides (E. coli, black bars; S. aueris, gray bars).

FIG. 24. CD spectra of (RW)ₙ—NH₂ (n=1, 2, 3, 4 and 5) peptides in aqueous solution (solid line), and in the presence of the model lipids systems POPC (dashed line), and POPG (dashed-dotted line), in 20 mM phosphate buffer (pH 7.4, 100 mM NaCl). Data are expressed as mean residue ellipticity.

FIG. 25. Relative fluorescence intensity of (RW)ₙ—NH₂ peptides in aqueous solution and the presence of SUV's (buffer, black bars; POPG, gray bars; POPC, white bars). Fluorescence intensity in aqueous solution was used as reference. The peptide and lipid concentrations were 10 μM and 500 μM, respectively, for a lipid-to-peptide ratio of 50.

FIG. 26. Correlation between antimicrobial activity and chain length of (RW)ₙ—NH₂ peptides. A power-law fit y=cx^{a} (a and c are constants) means that log(y)=log(c)+alog(x). Here we have log(IC₅₀)=log(c)+a log(n) (n=1, 2, 3, 4 and 5; IC₅₀ is replaced by HD₅₀ in the correlation for red blood cell). The a values are −4.5 (r²=0.93), −4.3 (r²=0.95) and −3.3(r²=0.92), for E. coli (solid ring), S. aureus (open ring) and red blood cell (triangle), respectively.

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an” and “the” include plural references unless the context clearly dictates otherwise. Thus, for example, references to “the method” includes one or more methods, and/or steps of the type described herein and/or which will become apparent to those persons skilled in the art upon reading this disclosure and so forth in their entirety.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entireties.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Definitions

The terms used herein have the meanings recognized and known to those of skill in the art, however, for convenience and completeness, when describing the compounds, pharmaceutical compositions containing such compounds and methods of using such compounds and compositions, the following terms have the following meanings unless otherwise indicated.

“PMA” is a polymer of maleic anhydride, and usually refers to a homopolymer thereof.

“EMA” is a polymer of ethylene and maleic achydride. Polymers of this type are of great value according to the present invention.

“EMA-type” polymer is defined hereinafter.

“EMA-peptide” or “EMA/peptide” is a copolymer of ethylene and maleic anhydride having the antimicrobial/antiviral/antifungal peptide monomer covalently bonded thereto. The product is the same whether the peptide is reacted directly with an anhydride group of the maleic anhydride homopolymer or ethylene-maleic anhydride copolymer, or with a carboxyl group thereof, whether or not using an intermediate activating mechanism for carboxyl groups of the polymer. Anhydride groups not participating in the reaction by which the product is produced in aqueous medium are present in the product as carboxyl or carboxylate groups. Such non-participating groups may, however, be converted to amide, imide, ester, et cetera, groups, as can be present in MA/EMA-type polymers, as hereinafter defined.

“Peptide” means a chain of amino acid residues having between 2 and about 100 amino acid residues, and includes peptides which are purified from naturally occurring products, or produced by synthetic or recombinant DNA methods. Amino acid chains having greater than about 100 amino acid residues if present herein, are referred to as polypeptides. Synthetic peptides, prepared using the well known techniques of solid phase, liquid phase, or peptide condensation techniques, or any combination thereof, can include natural and unnatural amino acids. “Unnatural amino acids” are those that are not included in the standard 20 known amino acids. The standard known amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, as noted above. “Unnatural amino acids” means amino acids and corresponding peptides that are synthesized from single amino acid starting materials. Such unnatural amino acids may be prepared and used individually in accordance with the present invention, or may be incorporated into existing proteins. This method may be used to create analogs with unnatural amino acids. A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). The unnatural amino acids may include for example, the D-form of a known amino acid or derivatives of such amino acids, such as for example L-ornithine, L-homocysteine, L-homoserine, L-citrulline, 3-sulfino-L-alanine, N-(L-arginino)succinate, 3,4-dihydroxy-L-phenylalanine, 3-iodo-L-tyrosine, 3,5-diiodo-L-tyrosine, triiodothyronine, L-thyroxine, L-selenocysteine, beta-alanine, N-(L-arginino)taurine, 4-aminobutylate, (R,S)-3-amino-2-methylpropanoate, D-alanine, D-valine, D-leucine, D-isoleucine, D-aspartate, D-asparagine, D-glutamate, D-glutamine, D-serine, D-threonine, D-methionine, D-cysteine, D-lysine, D-arginine, D-histidine, D-proline, D-phenylalanine, D-tyrosine, and D-tryptophan. The molecule described in the present application may have some of these unnatural amino acids, as well as other unusual amino acid derivatives. Amino acids used for peptide synthesis may be standard Boc (N-amino protected N_t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile N-amino protected 9-fluorenylmethoxycarbonyl (Fmoc) amino acids first described by Carpino and Han (1972, J. Org. Chem. 37:3403-3409). Thus, polypeptide of the invention may comprise D-amino acids, a combination of D- and L-amino acids, and various “designer” amino acids (e.g.,. -methyl amino acids, [symbol] -methyl amino acids, and N-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps,. -helices,. turns,. sheets,. -turns, and cyclic peptides can be generated.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Subject” includes humans. The terms “human,” “patient” and “subject” are used interchangeably herein.

“Therapeutically effective amount” means the amount of the complex, or the salivary agglutinin peptides and fragments, analogs, mimics, derivatives or variants of the invention, that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder. In the present invention, the treatments using the agents and compositions and/or means of delivery described may be provided to inhibit infectivity by a microbial agent, either of bacterial, viral or fungal origin. In one particular embodiment of the invention, the treatments may be provided to inhibit retrovirus infectivity, more particularly, to prevent the spread of HIV. The treating may also be for the purpose of reducing or diminishing the symptoms or progression of an immunodeficiency disease or to reduce the viral load in the patient. Furthermore, in treating a subject, the compounds of the invention may be administered to a subject already suffering from HIV or other retroviral disease as provided herein or to prevent or inhibit the spread of the disease or the occurrence of the symptoms or sequelae of such viral infection, including susceptibility to other infections such as cytomegalovirus infections or Kaposi's sarcoma.

“Analog” as used herein, refers to a chemical compound, a nucleotide, a protein, or a polypeptide that possesses similar or identical activity or function(s) as the chemical compounds, nucleotides, proteins or polypeptides having the desired activity and therapeutic effect of the present invention (e.g. to inhibit retrovirus infectivity, in particular, human immunodeficiency virus (HIV)), but need not necessarily comprise a compound or sequence that is similar or identical to those compounds or sequences of the preferred embodiment, such as that of SEQ ID NOS: 1, 2, 3, 4 and 7. As used herein, a nucleic acid or nucleotide sequence, or an amino acid sequence of a protein or polypeptide is “similar” to that of a nucleic acid, nucleotide or protein or polypeptide having the desired activity if it satisfies at least one of the following criteria: (a) the nucleic acid, nucleotide, protein or polypeptide has a sequence that is at least 70%, at least 75%, at least 80%, at least 85%, at least 90%, at least 95% or at least 99%) identical to the nucleic acid, nucleotide, protein or polypeptide sequences having the desired activity as described herein (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the AAPI; or (c) the polypeptide is encoded by a nucleotide sequence that is at least 70%, at least 75%, at least 80%, at least 85%, at least 90%, at least 95% or at least 99%) identical to the nucleotide sequence encoding the polypeptides of the present invention having the desired therapeutic effect. As used herein, a polypeptide with “similar structure” to that of the preferred embodiments of the invention refers to a polypeptide that has a similar secondary, tertiary or quaternary structure as that of the preferred embodiment (e.g. SEQ ID NOs: 1, 2, 3, 4 and 7). The structure of a polypeptide can determined by methods known to those skilled in the art, including but not limited to, X-ray crystallography, nuclear magnetic resonance, and circular dichroism.

“Derivative” refers to the chemical modification of molecules, either synthetic organic molecules or proteins, nucleic acids, or any class of small molecules such as fatty acids, or other small molecules that are prepared either synthetically or isolated from a natural source, such as a plant, that retain at least one function of the active parent molecule, but may be structurally different. Chemical modifications may include, for example, replacement of hydrogen by an alkyl, acyl, or amino group. It may also refer to chemically similar compounds which have been chemically altered to increase bioavailability, absorption, or to decrease toxicity. A derivative polypeptide is one modified by, for example, pegylation, or any similar process that retains at least one biological or immunological function of the polypeptide from which it was derived.

A “variant” of polynucleotides or polypeptides, as the term is used herein, are polynucleotides or polypeptides that are different from a reference polynucleotide or polypeptide, respectively. Variant polynucleotides are generally limited so that the nucleotide sequence of the reference and the variant are closely related overall and, in many regions, identical. Changes in the nucleotide sequence of the variant may be silent. That is, they may not alter the amino acid sequence encoded by the polynucleotide. Where alterations are limited to silent changes of this type a variant will encode a polypeptide with the same amino acid sequence as the reference. Alternatively, changes in the nucleotide sequence of the variant may alter the amino acid sequence of a polypeptide encoded by the reference polynucleotide. Such nucleotide changes may result in amino acid substitutions, additions, deletions, fusions, and truncations in the polypeptide encoded by the reference sequence. Variant polypeptides are generally limited so that the sequences of the reference and the variant are that are closely similar overall and, in many regions, identical. For example, a variant and reference polypeptide may differ in amino acid sequence by one or more substitutions, additions, deletions, fusions, and truncations, which may be present or absent in any combination. Such variants can differ in their amino acid composition (e.g. as a result of allelic or natural variation in the amino acid sequence, e.g. as a result of alternative mRNA or pre-mRNA processing, e.g. alternative splicing or limited proteolysis) and in addition, or in the alternative, may arise from differential post-translational modification (e.g., glycosylation, phosphorylation, isoprenylation, lipidation).

“Fragment” refers to either a protein or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide. In a preferred embodiment, the fragment possesses at least partial, or a low amount of the functional activity of the parent nucleic acid or protein.

The “percent identity” of two amino acid sequences or of two nucleic acid sequences can be or is generally determined by aligning the sequences for optimal comparison purposes (e.g., gaps can be introduced in either sequences for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

The terms “mimic” or “mimetic” are used interchangeably herein and the terms are used to describe a molecule, which may or may not be proteinaceous in nature and extends to analogs, derivatives or variants of soluble gp340 and fragments thereof, as exemplified in SEQ ID NO: 1, 2, 3 and 4. The “mimics” or “mimetics” mimic the functional activity of gp340 as related to inhibition of retrovirus infectivity or prevention of virus spread and these mimics or mimetics may be either naturally derived or synthetically prepared. A peptide which mimics gp340 is preferably at least three amino acids, although peptides of any length are within the scope of the invention. Accordingly, peptides and non-peptide mimics displaying substantially equivalent or altered activity are likewise contemplated. These modifications may be deliberate, for example, such as modifications obtained through site-directed mutagenesis, or may be accidental, such as those obtained through screening for mimics or mimetics using the methods and assays provided and described herein. Also, the terms mimic or mimetic are intended to include within their scope those peptides specifically recited herein as well as all variants, analogs and active fragments thereof, including substantially homologous variants and analogs. The identity or location of one or more amino acid residues may be changed or modified to include, for example, active fragments such as deletions containing less than all of the residues specified for the peptide, variants wherein one or more residues are replaced or substituted by other residues or wherein one or more amino acid residues are added to a terminal or medial portion of the peptide, and analogs wherein one or more residues are replaced or substituted with unnatural amino acids, L-amino acids, various “designer” amino acids (for example P-methyl amino acids, Ca methyl amino acids, and Na methyl amino acids), nonclassical amino acids or synthetic amino acids. Analogs further encompass cyclic peptides, which can be generated by any of recognized methods in the art.

The amino acid residues described herein are preferred to be in the “L” isomeric form. However, residues in the “D” isomeric form can be substituted for any L-amino acid residue, as long as the desired functional property, for example, anti-retroviral activity, is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table 1 of Correspondence:

TABLE 1 OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

As used herein, “RW” refers to peptides wherein R (arginine) and W (tryptophan) are present as a single unit or as multiple units, and in any orientation, for example, RW, WR, RWRW, WRWR, RRWW, WWRR, RWWR, WRRW, (RW)ₙ, (WR)ₙ, (WRWR)ₙ, (RWRW)ₙ, (RRWW)ₙ, (RWWR)ₙ, (WRRW)ₙ, (WWRR)ₙ. N may be from about 2 to about 20, preferably about 4 to about 15, and more preferably about 5 to 10.

In addition, the present invention envisions preparing peptides that have more well defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a molecule would be resistant to peptide bond hydrolysis, e.g., protease activity. Such peptides would provide ligands with unique function and activity, such as extended half-lives in vivo due to resistance to metabolic breakdown, or protease activity. Furthermore, it is well known that in certain systems constrained peptides show enhanced functional activity (Hruby, 1982, Life Sciences 31:189-199; Hruby et al., 1990, Biochem J. 268:249-262); the present invention provides a method to produce a constrained peptide that incorporates random sequences at all other positions.

A “constrained”, “cyclic” or “rigidized” peptide may be prepared synthetically, provided that in at least two positions in the sequence of the peptide an amino acid or amino acid analog is inserted that provides a chemical functional group capable of cross-linking to constrain, cyclize or rigidize the peptide after treatment to form the cross-link. Cyclization will be favored when a turn-inducing amino acid is incorporated. Examples of amino acids capable of cross-linking a peptide are cysteine to form disulfide, aspartic acid to form a lactone or a lactase, and a chelator such as α-carboxyl-glutamic acid (Gla) (Bachem) to chelate a transition metal and form a cross-link. Protected α-carboxyl glutamic acid may be prepared by modifying the synthesis described by Zee-Cheng and Olson (1980, Biophys. Biochem. Res. Commun. 94:1128-1132). A peptide in which the peptide sequence comprises at least two amino acids capable of cross-linking may be treated, e.g., by oxidation of cysteine residues to form a disulfide or addition of a metal ion to form a chelate, so as to cross-link the peptide and form a constrained, cyclic or rigidized peptide.

“Contacting”, as used herein, refers to the interaction between the gp340 protein or fragment, analog, variant or derivative thereof and the gp120 envelope of a retrovirus for a sufficient period of time to permit binding of the gp340 protein or fragment, analog, variant or derivative thereof to the gp120 envelope of the retrovirus virion, as can be easily measured by methods routine in the art.

“Phage display library”, as used herein, refers to a multiplicity of phage which express random amino acid sequences of less than 100 amino acids in length, less than 75 amino acids, less than 50 amino acids, and particularly within the range of 3 to 25 amino acids at a location which may be bound by an antibody.

A “small molecule” refers to a composition that has a molecular weight of less than 3 kilodaltons (kDa), and preferably less than 1.5 kilodaltons, and more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodaltons, and preferably less than 1.5 kilodaltons, and more preferably less than about 1 kDa.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

An individual “at risk” may or may not have detectable disease, and may or may not have displayed detectable disease prior to the treatment methods described herein. “At risk” denotes that an individual who is determined to be more likely to develop a symptom or disease based on conventional risk assessment methods or has one or more risk factors that correlate with development of an immunodeficiency disease, for example, HIV or sequelae associated with HIV, such as risk for developing other infections. An individual having one or more of these risk factors has a higher probability of acquiring or developing an immunodeficiency disease than an individual without these risk factors.

As used herein, the term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” as used herein is a compound, such as a protein or polypeptide, which is homogeneously pure, for example, where at least 95% of the total protein (by volume, by wet or dry weight, or by mole percent or mole fraction) in a sample is the protein or polypeptide of interest.

As used herein, “parenteral” administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

A “conservative amino acid substitution” refers to the substitution of one or more of the amino acid residues of a protein with other amino acid residues having similar physical and/or chemical properties. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

As used herein, the term “specific binding” or “specifically binds” means a protein, such as a glycoprotein, which recognizes and binds the gp120 protein of HIV, but does not substantially recognize or bind other molecules in a sample. The term also means an antibody which recognizes and binds a salivary glycoprotein or a ligand thereof, but does not substantially recognize or bind other molecules in a sample.

General Description

The Complexes

As set forth herein, the polymer-peptide monomer complexes comprise antimicrobial/antiviral/antifungal peptide monomers covalently bound to a polymer backbone. The peptide component of the complex may be and include any sequence of amino acids that has a lethal effect on bacteria, viruses or fungi in its monomeric form. More particularly, the peptides may be any antimicrobial peptides, including natural products found in organisms, fragments of natural peptides, and any synthetic analogs or de novo designs. These peptides can accordingly include nonnatural amino acids: beta-amino acids, d-amino acids and/or non-indigenous amino acids.

Numerous antimicrobial and antiviral peptides have been identified and are known, and representative peptides may be selected from Antiviral protein Y3, Alloferon 1, Lactoferricin B, hexapeptide, Tricyclic peptide RP, indolicidin, GNCP-1, GNCP-2, HNP-1 Defensin, HNP-2 Defensin, HNP-3 Defensin, CORTICOSTATIN III (MCP-1), CORTICOSTATIN IV (MCP-2), NP-3A defensin, Protegrin 2, Protegrin 3, Protegrin 4, Protegrin 5, RatNP-1, RatNP-2, RatNP-3, RatNP-4, Caerin 1.1, Circulin A (CIRA), Circulin B (CIRB), Cyclopsychotride A (CPT), Ginkobilobin, and Alpha-basrubrin. The forgoing representative listing is taken from (http://aps.unmc.edu/AP/database/antiV.php) and the content and disclosure of this site is incorporated herein by reference in its entirety. Representative peptides listed at the site are set forth herein by way of illustration and not limitation, and the present invention is considered to include the use of all such peptides within its scope. In one particular embodiment of the invention, the peptides are selected from the “RW” peptides described herein. In another particular embodiment, the peptides may be derived from salivary agglutinin protein, including fragments, analogs, mimics, derivatives or variants thereof, including, but not limited to, those amino acid sequences set forth in SEQ ID NOs: 1, 2, 3, 4 and 7. In addition to the peptides just listed, peptides may be derived from de novo synthesis, such as from the preparation of unnatural amino acids, and the like, and the present invention is intended to include all such peptides and like materials within its scope.

Suitable polymers may include linear polymers or copolymers such as those described in greater detail hereinafter, or nonlinear structures, such as dendrimers. Any molecule with multiple reactive sites can be a suitable scaffold for the peptide monomers that are a part of the complexes of the invention. In a particular embodiment, the polymers may preferably contain carboxyl or anhydride linkages, especially where the peptide contains an amino, hydroxyl (including phenolic hydroxyl), or sulfhydryl group not essential for its activity. Where the peptide contains a carboxyl group not essential for activity, the polymer can contain free hydroxyl or amine groups for reaction, therewith. The polymer is preferably EMA or an EMA-type polymer, but it can be any of those types disclosed for coupling or reaction with a peptide, and in any event it is adapted to effect covalent bonding with the peptide to produce a peptide-polymer product either directly or indirectly using an activating agent. Inasmuch as the antimicrobial/antiviral/antifungal activity of the starting peptide is desired to be not only retained but significantly increased in the final product by several orders of magnitude over the peptide alone, it is, of course, firstly necessary that bonding of the peptide to the polymer be through a group which will not result in inactivation of an active site on the peptide. Among the various reactive groups of peptides may be mentioned, besides amino and sulfhydryl, also hydroxyl (including phenolic hydroxyl), carboxyl and imidazolyl. Such groups are present in free or unbound form in inactive portions of peptide molecules, as in a lysine, cysteine, serine, aspartic acid, glutamic acid, threonine, histidine, or tyrosine moiety, where the particular moiety in question is not considered essential for activity. Therefore, attachment to the polymer molecule is through reaction of the polymer with such groups so as to avoid inactivation of the peptide during attachment to the polymer molecule.

Generally, the linkage is an amide, imide, ester, thioester, or disulfide group, such as formed by the carboxyl or anhydride of the polymer with an amine or hydroxyl group in a non-essential moiety of the peptide chain. Amides are formed by reacting pendant amino groups of the peptide with carboxylic anhydride groups of the carrier polymer in water, in aqueous buffer media, or in mixed solvents. Amides, imides and esters are readily formed by activating carboxyl groups of the polymer, or alternatively pendant carboxyls of the peptide, and reacting them with respective hydroxyl, amine or mercaptan groups on the other reactant. Such activation may tie effected using various carbodiimides, carbodiimidazoles, Woodward's or Sheehan's reagent, or the like, to form highly active intermediates capable of reacting with other groups mentioned above under mild conditions, the latter favoring retention of relevant peptidic activity.

The polymer selected for such reaction can therefore be said to be adapted to couple or react with the peptide, either directly or indirectly through use of an activating agent, as already indicated, and in any event to effect covalent bonding with the peptide. The attachment procedures given are conducted by techniques adapted to include any requisite protection for the peptide, which may include a reversible blocking of any active site or sites.

In its broadest context, the polymer to which the peptides are coupled contains carboxyl or anhydride linkages, especially where the peptides contain an amino, hydroxyl, or sulfhydryl group not essential for their activity. Where a peptide contains a carboxyl group not essential for activity, the polymer can contain hydroxyl or amine groups for reaction herewith. The polymer may be EMA or an EMA-type polymer, or be any of those types previously disclosed for coupling or reaction with the peptide component, and in any event it is adapted to couple or react with the peptides to effect covalent bonding and production of the desired monomeric peptide-polymer complex or product.

Since covalent bonding is desired, it is understood that the carrier polymer is tailored to contain at least one reactive site for each polymer molecule with which the peptides can react, either directly or indirectly, to produce a covalent bond. According to the instant invention, this reactive site (or sites) is preferably a carboxyl or carboxylic anhydride group.

In a particular embodiment, the polymeric reactant is a polymer (a) comprising chains of carboxylic acid or carboxylic acid anhydride units, or (b) comprising units of carboxylic acid or carboxylic acid anhydride groups separated by carbon chains of at least one and not more than four carbon atoms, said carbon chains being part of a unit which contains a maximum of eighteen carbon atoms, said polymers being formed by polymerization of polymerizable acids or anhydrides or by copolymerizing a polymerizable acid or anhydride with another copolymerizable monomer, and preferably wherein the starting acid or anhydride and any additional copolymerizable monomer are unsaturated and such polymerization or copolymerization comprises addition type polymerization or copolymerization involving such unsaturation.

Among the polymers suitable for the practice of the instant invention, polymeric polyelectrolytes having units of the formula 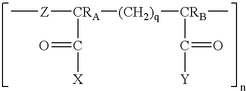
wherein:
R_{A} and R_{B} are selected from the group consisting of hydrogen, halogen (preferably chlorine), alkyl of 1 to 4 carbon atoms (preferably methyl), cyano, phenyl, or mixtures thereof; provided that not more than one of R_{A} and R_{B} is phenyl;
Z is a bivalent radical (preferably alkylene, phenylalkylene, lower-alkoxyalkylene, and lower-aliphatic acyloxyalkylene) of 1 to 18 carbon atoms, inclusive, and preferably comprising a bivalent carbon chain having 1 to 4 carbon atoms, inclusive, said carbon chain being a part of a unit which contains 1 to 18 carbon atoms, inclusive,
q is zero or one,
X and Y are selected from hydroxy, —O alkali metal, OR, —OH, —NH₃—OH, —R₃N, —OH, —R₂NH, —OH—RNH₂, —NRR′, —(O)ₚ—W—(NR′R′)ₓ, and —(O)ₚ—W—(—OH)ₓ, wherein x is 1 to 4 and p is zero or one, wherein R is selected from the group consisting of alkyl, phenylalkyl, or phenyl, in each case of 1 to 18 carbon atoms, wherein R′ is H or R, wherein Q is oxygen or —NR′—, and wherein W is a bivalent radical preferably selected from lower-alkylene, phenyl, phenylalkyl, phenylalkylphenyl, and alkylphenylalkyl having up to 20 carbon atoms, X and Y taken together can be an oxygen atom, and at least one of X and Y being hydroxyl or X and Y together constituting an oxygen atom, are preferred.

Many of these polymers are commercially available and others are simple derivatives of commercially available products, which can be readily prepared either prior to or simultaneously with the hormone attachment reaction, or produced as a minor modification of the basic polymer after attachment. Such polymers containing the above-described EMA-type units are hereinafter referred to as an “EMA-type polymer.”

As pointed out hereinafter, preferably the units of the formula given are recurring, n being at least 8. When the units are recurring, the symbols in the various recur-ring units do not necessarily stand for the same thing in all of the recurring units. Moreover, where the units are recurring, some of the X and Y groups may have meanings besides hydroxy or oxygen. For example, some, but not all, of them may be present in the form of imide groups, that is, groups in which X and Y together are —NR— or —N—W—(—NR′R′)ₓ, wherein R, W and R′ have the values previously assigned. A preferred type of polymeric material useful in the practice of the invention is the polymer of an olefinically unsaturated polycarboxylic acid or derivative with itself or in approximately equimolar proportions with at least one other monomer copolymerizable therewith. The polycarboxylic acid derivative can be of the non-vicinal type, including acrylic acid, acrylic anhydride, methacrylic acid, crotonic acid or their respective derivatives, including partial salts, amides and esters or of the vicinal type, including maleic, itaconic, citraconic, α,α-dimethyl maleic, α-butyl maleic, α-phenyl maleic, fumaric, aconitic, α-chloromaleic, α-bromomaleic, α-cyanomaleic acids including their partial salts, amides and esters. Anhydrides of any of the foregoing acids are advantageously employed.

Co-monomers suitable for use with the above functional monomers include α-olefins such as ethylene, propylene, isobutylene, 1- or 2-butene, 1-hexene, 1-octene, 1-decene, 1-dodecene, 1-octadecene, and other vinyl monomers such as styrene, α-methyl styrene, vinyl toluene, vinyl acetate, vinyl amine, vinyl chloride, vinyl formate, vinyl propionate, vinyl alkyl ethers, e.g., methylvinylether, alkyl acrylate, alkyl methacrylates, acrylamides and alkylacryl amides, or mixtures of these monomers. Reactivity of some functional groups in the copolymers resulting from some of these monomers permits formation of other useful functional groups in the formed copolymer including hydroxy, lactone, amine and lactam groups.

Any of the polybasic acid derivatives may be copolymerized with any of the other monomers described above, and any other monomer which forms a copolymer with dibasic acid derivatives. As an additional modification, the polybasic acid derivatives can be copolymers with a plurality of comonomers, in which case the total amount of the comonomers will preferably be about equimolar with respect to the polybasic acid derivatives. Although these copolymers can be prepared by direct polymerization of the various monomers, frequently they are more easily prepared by an after-reaction modification of an existing copolymer.

Copolymers of anhydrides and another monomer can be converted to carboxyl-containing copolymers by reaction with water, and to ammonium, alkali and alkaline earth metal and alkylamine salts thereof by reaction with alkali metal compounds, alkaline earth metal compounds, amines or ammonia, either prior to, during, or subsequent to peptide attachment, etc. Other suitable derivatives of the above polymers include the partial alkyl or other esters and partial amides, alkyl amides, dialkyl amides, phenyl alkyl amides or phenyl amides prepared by reacting carboxyl groups on the polymer chain with the selected amines or alkyl or phenylalkyl alcohol as well as amino esters, amino amides, hydroxy amides and hydroxy esters, wherein the functional groups are separated by lower alkylene, phenyl, phenylalkyl, phenylalkylphenyl, or alkylphenylalkyl, which are prepared in the same manner in each case with due consideration of preservation of peptide attachment sites as previously stated. Other aryl groups may be present in place of phenyl groups. Particularly useful derivatives are those in which negatively charged carboxyl groups are partially replaced with amine or amine salt groups. These are formed by reaction of said carboxyls with polyamines such as dimethylamino propylamine or dialkylaminoalcohols such as dimethylaminoethanol, the former forming an amide linkage with the polymer and the latter an ester linkage. Suitable selection of the above derivatives permits control of several parameters of performance for the peptide-polymer complexes of the invention.

Representative dibasic acid or anhydride-olefin polymers, especially maleic acid or anhydride-olefin polymers, of the foregoing type (EMA-type) are known, for example, from U.S. Pat. Nos. 2,378,629, 2,396,785, 3,157,595 and 3,340,680. Generally, the copolymers are prepared by reacting ethylene or other unsaturated monomer or mixtures thereof, as previously described, with the acid anhydride in the presence of a peroxide catalyst in an aliphatic or aromatic hydrocarbon solvent for the monomers but nonsolvent for the interpolymer formed. Suitable solvents include benzene, toluene, xylene, chlorinated benzene and the like. While benzoyl peroxide is usually the preferred catalyst, other peroxides such as acetyl peroxide, butyryl peroxide, di-tertiary butyl peroxide, lauroyl peroxide and the like, or any of the numerous azo catalysts, are satisfactory since they are soluble in organic solvents. The copolymer preferably contains substantially equimolar quantities of the olefin residue and the anhydride residue. Generally, it will have a degree of polymerization of 8 to 10,000, preferably about 100 to 5,000, and a molecular weight of about 1,000 to 1,000,000, preferably about 10,000 to 500,000. The properties of the polymer, such as molecular weight, for example, are regulated by proper choice of the catalyst and control of one or more of the variables such as ratio of reactants, temperature, and catalyst concentration or the addition of regulating chain transfer agents, such as diisopropyl benzene; propionic acid, alkyl aldehydes, or the like. The product is obtained in solid form and is recovered by filtration, centrifugation or the like. Removal of any residual or adherent solvent can be effected by evaporation using moderate heating. Numerous of these polymers are commercially available. Particularly valuable copolymers are homopolymers of maleic anhydride (PMA), and those derived from ethylene and maleic anhydride (PEMA) in approximately equimolar proportions. Both products are commercially available.

The maleic anhydride homopolymers and copolymers thus obtained have repeating anhydride linkages in the molecule, which are readily hydrolyzed by water to yield the acid form of the copolymer, rate of hydrolysis being proportional to temperature. In view of the fact that the attachment or coupling reactions of the present invention are carried out in aqueous solutions or suspensions, or using water-solvent mixtures, the product of the covalent bonding of the hormone to PMA or PEMA has carboxyl or carboxylate groups attached to its chains adjacent the attached hormone in stead of anhydride groups, due to hydrolysis of the anhydride groups, which do not react with the hormone, during the reaction. The same is true of nonreacting anhydride groups present in other polymers, such as EMA type polymers, which hydrolyze to carboxyl or carboxylate groups during the reaction.

In certain aspects and where appropriate, the present invention extends to the preparation of prodrugs and derivatives of the complexes of the invention. Prodrugs are derivatives which have cleavable groups and become by solvolysis or under physiological conditions the peptide components of the complexes of the invention, which are pharmaceutically active, in vivo.

Pharmaceutical Compositions and Formulations Comprising the Complexes of the Invention

When employed as pharmaceuticals, the polymer-peptide complexes of this invention are typically administered in the form of a pharmaceutical composition. Such compositions can be prepared in a manner well known in the pharmaceutical art and comprise at least one active complex.

Generally, the complex of this invention is administered in a pharmaceutically effective amount. The amount of the complex actually administered will typically be determined by a physician, in the light of the relevant circumstances, including the condition to be treated, the chosen route of administration, the actual complex administered, the age, weight, and response of the individual patient, the severity of the patient's symptoms, and the like.

The pharmaceutical compositions of this invention can be administered by a variety of routes including by way of non limiting example, oral, rectal, transdermal, subcutaneous, intravenous, intramuscular and intranasal. Depending upon the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions of this invention. The present invention, however, is not limited to the following pharmaceutical compositions.

### Formulation 1—Tablets

A compound containing a complex of the invention is admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

### Formulation 2—Capsules

A compound containing a complex of the invention is admixed as a dry powder with a starch diluent in an approximate 1:1 weight ratio. The mixture is filled into 250 mg capsules (125 mg of active compound per capsule).

### Formulation 3—Liquid

A compound containing a complex of the invention (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4—Tablets

A compound containing a complex of the invention is admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5—Injection

A compound containing a complex of the invention is dissolved or suspended in a buffered sterile saline injectable aqueous medium to a concentration of approximately 5 mg/ml.

### Formulation 6—Topical

Stearyl alcohol (250 g) and a white petrolatum (250 g) are melted at about 75° C. and then a mixture of a compound of formula 1 (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

Methods of Treatment

The present complexes may be used as therapeutic agents for the treatment of conditions in mammals. Accordingly, the complexes and pharmaceutical compositions of this invention find use as therapeutics for preventing and/or treating infections and like maladies resulting from bacterial, viral or fungal attack, and related conditions in mammals including humans.

In a method of treatment aspect, this invention provides a method of treating a mammal susceptible to or afflicted with a condition associated with or resulting from bacterial, viral or fungal attack or infection, which method comprises administering an effective amount of one or more of the pharmaceutical compositions just described.

In additional method of treatment aspects, this invention provides methods of treating a mammal susceptible to or afflicted with a variety of bacteria or other infections, including strains which have developed resistance to traditional antibiotics, such as, for example, Staphylococcus aureus. The method comprises administering an effective condition-treating or condition-preventing amount of one or more of the pharmaceutical compositions just described.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as viral or microbial conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound or its derivative, with preferred doses each providing from about 0.1 to about 10 mg/kg and especially about 1 to about 5 mg/kg.

Transdermal doses are generally selected to provide similar or lower blood levels than are achieved using injection doses.

The complexes of this invention can be administered as the sole active agent or they can be administered in combination with other agents, including other active derivatives.

General Synthetic Procedures

The complexes of this invention can be prepared from readily available starting materials using the general methods and procedures described earlier and illustrated schematically in the examples that follow. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

Description of Exemplary Microbicides for Use in the Invention

It is envisioned that the complexes of the invention may be used with any microbicide, in particular, those having anti-bacterial activity, as well as, anti-viral activity or anti-fungal activity. While advances in the design and development of microbicides that have anti-HIV activity have accelerated, currently the 5 candidate microbicides in Phase 3 clinical trials belong to only 2 chemical classes: surface active agents and high molecular weight sulfated polymers. Closely behind in development are a variety of agents, some of which are based on systemic drugs that target key steps in the HIV life cycle, e.g., reverse transcriptase or entry inhibitors (Wilkins, C., Dishongh, R, Moore, S. C., Whitt, M. A, Chow, M. and Machaca, K. (2005). RNA interference is an antiviral defence mechanism in Caenorhabditis elegans. Nature 436(7053): 1044-1047).

The innate immune system, a first line of defense against invading pathogens, plays a dominant role at the mucosal surface (Levy, J. A (2001). The importance of the innate immune system in controlling HIV infection and disease. Trends Immunol 22(6): 312-316). Amongst the soluble components of this system are gp340, cytokines, chemokines, lectin-binding proteins, defensins, and complement. gp340 was originally identified as a salivary agglutinin for bacteria (Demuth, D. R., Davis, C. A, Corner, A M., Lamont, R. J., Leboy, P. S. and Malamud, D. (1988). Cloning and expression of a Streptococcus sanguis surface antigen that interacts with a human salivary agglutinin. Infect Immun 56(9): 2484-2490; Prakobphol A, Feng X, Hoang V M, et al.: Salivary agglutinin, which binds to S. mutans and H. pylori, is the lung scavenger receptor cysteine-rich protein gp340. J. Biol. Chem. 2000; 275:39860-39866), and later identified as a member of the scavenger-receptor cysteine rich (SRCR) superfamily, an alternatively spliced form of the tumor suppressor protein DMBT1 (Mollenhauer J, Wiemann S, Scheurlen W, et al.: DMBT-1, a new member of the SRCR superfamily, on chromosome 10q25.3-26.1 is deleted in malignant brain tumours. Nut Genet 1997; 17:32-39). gp340 is found in tears, saliva, bronchial alveolar fluid (Bikker F J, Ligtenberg A J, van der Wal J E, et al.: Immunohistochemical detection of salivary agglutininlgp-340 in human parotid, submandibular, and labial salivary glands. J Dent Res 2002; 81: 134-139; Schulz, B. L., Oxley, D., Packer, N. H. and Karlsson, N. G. (2002). Identification of two highly sialylated human tear-fluid DMBT1 isoforms: the major high-molecular-mass glycoproteins in human tears. Biochem J 366(Pt 2): 511-520), where it may function as a modulator of microbial infection, and also in trachea, and small intestine (Holmskov U, Mollenhauer J, Madsen J, et al.: Cloning of gp340, a putative opsonin for surfactant protein D. Proc. Natl. Acad. Sci. 1999; 96: 10794-10799) and on the surface of cells in the female reproductive tract where it may play other functional roles.

Previous studies using the human salivary agglutinin (SAG/gp-340) demonstrated its anti-HIV activity. The present studies were conducted to determine which portion of the SAG molecule was responsible for binding to gp120, thereby interfering or preventing the infectivity of HIV-1. In a particular embodiment, it was determined that the HIV-1 inhibitory activity of SAG is mediated by interaction with a highly conserved sequence located at the N-terminus of the viral envelop V3 loop. A number of amino acids within this V3 sequence are also involved in gp120 binding to CCR5, a co-receptor for HIV-1 and point mutation of these residues decreased or abolished viral infectivity. Due to the broad specificity of gp-340 for both CCR5 and CXCR4 viruses and the highly conserved nature of the gp120 sequence involved with binding, gp-340 is an interesting candidate for further exploration as an anti-HIV-1 agent. SAG is identical to gp-340, an alternatively spliced form of DMBT-1 (Holmskov U, Mollenhauer J, Madsen J, et al.: Cloning of gp340, a putative opsonin for surfactant protein D. Proc. Natl. Acad. Sci. 1999; 96: 10794-10799; Prakobphol A, Feng X, Hoang V M, et al.: Salivary agglutinin, which binds to S. mutans and H. pylori, is the lung scavenger receptor cysteine-rich protein gp340. J. Biol. Chem. 2000; 275:39860-39866; Ligtenberg T J M, Bikker F J, Groenink J, et al.: Human salivary agglutinin binds to lung surfactant protein-D and is identical with scavenger receptor protein gp340. Biochem. J. 2001; 359:243-248), a gene initially identified as a brain tumor suppressor and more recently found to be present ubiquitously on many mucosal surfaces, including lung, trachea, salivary gland, and the gastrointestinal tract, for example, the small intestines. Gp-340 exists in both cell membrane-bound and secreted forms. The cell-associated form has been identified in many cell types with immune or host defense functions, including epithelial cells and alveolar macrophages. Gp-340 is believed to play important roles in innate immunity by acting as an opsonin receptor for surfactant proteins A and D in the lung. This binding enhances phagocytosis and killing of pathogenic microorganisms by neutrophils and alveolar macrophages.

AIDS Research and Human Retroviruses

The secreted form of gp-340 agglutinates bacteria (Ericson T and Rundegren J: Characterization of a salivary agglutinin reacting with a serotype c strain of Streptococcus mutans. Eur. J. Biochem. 1975; 133:255-261) and is thought to play important roles in innate immune defense (Resnick D, Pearson A and Krieger M: The SRCR superfamily: a family reminiscent of the Ig superfamily. Trends Biochem Sci 1994; 195-8). For purposes of this invention, the salivary agglutinin or SAG, is used interchangeably with gp340 and DMBT-1. DMBT-1 encodes a protein with 14 highly homologous SRCR domains separated by SRCR-interspersed domains (SID), two CUB domains, and a ZP domain at the C-terminus (Holmskov U, Mollenhauer J, Madsen J, et al.: Cloning of gp340, a putative opsonin for surfactant protein D. Proc. Natl. Acad. Sci. 1999; 96: 10794-10799). Each SRCR domain contains 100-110 amino acids rich in cysteines with characteristic spacing. Sequence analysis showed that the first 13 SRCRs share 88-100% sequence identity, and the 14 SRCRs shares 65% identity with the others. All gp-340 SRCRs contain 8 cysteines absolutely conserved in both numbers and positions, suggesting their importance for structural integrity. Mature gp-340 is a highly glycosylated protein with an apparent molecular weight of 340 kDa, almost half of the mass contributed by glycans (Demuth D R, Larnmey M S, Huck M, Lally E T and Malamud D: Comparison of Streptococcus mutans and Streptococcus sanguis receptors for human salivary agglutinin. Microb Pathog 1990; 9: 199-211; Demuth D R, Golub E E and Malamud D: Streptococcal-host intractions: Structural and functional analysis of a S. sanguis receptor for a human salivary glycoprotein. J Biol Chem 1990:265:7 120-7 126). Ten out of 14 potential N-glycosylation sites are located in the non-SRCR sequences near the C-terminus of the protein. SRCR-4, -6, -13 and -14 each has one potential N-glycosylation site (Holmskov U, Mollenhauer J, Madsen J, et al.: Cloning of gp340, a putative opsonin for surfactant protein D. Proc. Natl. Acad. Sci. 1999; 96: 10794-10799). There are also a number of potential sites for O-linked glycosylation but the extent of their usage is not known. Functional studies suggested that SAG and gp-340 exhibit similar in vitro biologic activities (Ligtenberg T J M, Bikker F J, Groenink J, et al.: Human salivary agglutinin binds to lung surfactant protein-D and is identical with scavenger receptor protein gp340. Biochem. J. 2001; 359:243-248). The salivary glycoprotein agglutinates S. mutans, the causative agent of dental caries (Demuth D R, Larnmey M S, Huck M, Lally E T and Malamud D: Comparison of Streptococcus mutans and Streptococcus sanguis receptors for human salivary agglutinin. Microb Pathog 1990; 9: 199-211) and both carbohydrates and amino acid sequences have been implicated in the bacterial aggregation (Demuth D R, Golub E E and Malamud D: Streptococcal-host intractions: Structural and functional analysis of a S. sanguis receptor for a human salivary glycoprotein. J Biol Chem 1990:265:7 120-7 126). In addition to S. mutans, a variety of other oral and non-oral bacteria are agglutinated by SAG, including S. oralis, S. gordonii, S. aureus and H. pylori. Although the anti-HIV-1 activity of SAG is well documented (Nagashunmugam T, Friedman H M, Davis C, Kennedy S, Goldstein L T and Malamud D: Human submandibular saliva specifically inhibits HIV-1. AIDS Res. Human Retro. 13 1997; 371-376; Wu Z W, Van Ryke D, Davis C, et al.: Salivary agglutinin (SAG) inhibits HIV-1 infectivity through interaction with the envelope glycoprotein. AIDS Res and Human Retroviruses 2003; 19:201-209; Wu Z W, Golub E, Abrams W R and Malamud D: gp340 (SAG) binds to the V3 sequence of gp120 important for chemokine receptor interaction. AIDS Res and Human Retroviruses 2004; 20:600-607), the anti-HIV-1 activity of recombinant gp-340 has not been reported. Direct evidence is lacking on which domains of gp-340 are involved in interacting with HIV-1. However, Bikker et al. (Bikker F J, Ligtenberg A J, van der Wal J E, et al.: Immunohistochemical detection of salivary agglutininlgp-340 in human parotid, submandibular, and labial salivary glands. J Dent Res 2002; 81: 134-139; Bikker F J, Ligtenberg A J M, End C, et al.: Bacteria binding by DMBTIISAGI gp-340 is confined to the VEVLXXXXW motif in its scavenger receptor cysteine rich domains. J Biol. Chem. 2004; 279:47699-47703) recently identified a 16-mer peptide derived from a consensus SRCR sequence that agglutinated S. mutans and other bacteria. The studies presented herein demonstrated the anti-HIV-1 activities of the recombinant SRCR protein expressed in a mammalian expression system. Furthermore, these studies also show that the N-terminal SRCR recombinant protein displays many properties of its parental gp-340. In addition, an 11 and 16 amino acid protein has been identified as having low level of anti-HIV activity. Methods are described here to stabilize these peptides having anti-HIV activity.

Moreover, the studies presented here demonstrated that soluble gp340 efficiently blocks HIV-1 infection in vitro by binding to a unique sequence in the HIV-1 Env. gp340 has been identified on the cell surface of vaginal and cervical cells where it can bind HIV and deliver infectious virus to CD4 positive cells (Weissman, Unpublished results). This cell surface interaction of HIV with gp340 may be similar to CD4 or DC-SIGN, since soluble CD4 can act as an HIV inhibitor, while cell surface CD4 serves as a receptor that promotes viral infection and whereas DC-SIGN on the cell surface, like gp340, can bind HIV and transmit infectious virus to susceptible target cells.

While a drug based on a 340,000 Dalton glycoprotein might appear to be a futile exercise, we have localized the active HIV-1 binding site of gp340 to a conserved peptide sequence that is amenable to development as a microbicide. Accordingly, the present invention relates to the development of analogs or derivatives of the soluble, inhibitory form of gp340, which when used as a microbicide, will inhibit HIV infectivity by neutralizing Env and which could also block interaction of virus with genital epithelium by competing with surface expressed gp-340. Moreover, the invention relates to the generation of a series of small molecular HIV inhibitors, or multimeric complexes of these inhibitors, and evaluates their bioactivities based on efficacy and cytotoxicity.

In one aspect, the invention provides fragments of the salivary agglutinin protein, gp340, (SEQ ID NO: 5) which exhibit anti-retroviral activity. More particularly, peptides comprising the amino acid sequences of SEQ ID NO: 1, 2, 3, 4 and 7 have been shown to inhibit the infectivity of HIV and thus prevent its spread. Given the size of the smaller fragments of gp340, the present invention also provides methods of stabilizing the active anti-HIV peptide fragments through particular chemical modifications or by attaching the peptides or analogs thereof to a biocompatible polymeric scaffold or presenting the peptides or fragments by way of a dendrimeric complex. Such modifications may provide for prevention of enzymatic breakdown of the peptides and thus aid in enhancing the anti-viral activity by prolonging the half-life of the proteins. The fragments or analogs or mimics thereof may be formulated in a pharmaceutical compositions for delivery via the oral or parenteral routes. They may be formulated for topical delivery, more particularly, for delivery to a mucosal surface, including the oral mucosa, the vaginal mucosa or the rectal mucosa. The soluble gp340 peptide fragments and pharmaceutical compositions comprising these fragments or mimics thereof may be administered prophylactically or therapeutically to a person suffering from an HIV infection or to a person at risk for acquiring an HIV infection. Since the peptides obtained from gp340 bind to the gp120 envelope of HIV, it is also contemplated that these peptides or derivatives, fragments or mimics thereof may be used to purify blood products to eliminate any possible contamination of the blood product with a retrovirus prior to use of the blood product for transfusions. Alternatively, the peptides, or fragments or derivatives or mimics thereof may be used in plasmapheresis procedures to clear the blood of patients having or suspected of having a retroviral infection.

Compositions comprising substantially purified gp340 peptides, derivatives, fragments or mimics thereof may include gp340 alone, or in combination with other salivary proteins or other proteins. gp340 peptides, derivatives, fragments or mimics thereof may be substantially purified by any of the methods well known to those skilled in the art. Substantially pure peptides may be purified by following known procedures for protein purification, wherein an immunological, chromatographic, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The gp340 peptides, or derivatives, fragments or mimics thereof, of the invention are, in one embodiment, a component of a pharmaceutical composition which may also comprise buffers, salts, other proteins, and other ingredients acceptable as a pharmaceutical composition. These compositions may be used in a method for prevention of HIV-1 infection of a patient or in the inhibition of HIV-1 infectivity of biological fluids. For example, the gp340 peptides may be used in lieu of the entire gp340 protein in a pharmaceutical composition. Use of the peptides, or derivatives, fragments or mimics thereof instead of the full gp340 protein results in simplified synthesis and purification procedures in the preparation of a pharmaceutical composition for use as a treatment against HIV-1 infection.

The peptides may be obtained from any portion of the gp340 salivary agglutinin protein that demonstrates specific binding with gp120 protein of HIV-1. However, the studies presented herein show particular peptides having anti-HIV activity. The sequences for these exemplary anti-HIV peptides derived from the full length salivary agglutinin or gp340 protein, are set forth in SEQ ID NOS: 1, 2, 3, 4 and 7. The HIV inhibitory effects of these peptides can be assessed as described in the assays presented in the Experimental Examples section herein.

The peptides, derivatives, fragments or mimics thereof may be used alone to inhibit HIV infectivity or may include other components, such as other proteins or polymers which may be covalently linked to the peptide, or may be non-covalently associated with the peptide or the peptides may be combined with other polymeric scaffolds or presented as a dendrimeric complex. The peptides may be used as monomers or multimers.

The peptides, derivatives, or fragments thereof may be generated by enzymatic digestion of the full gp340 protein. Enzymatic digestion methods may include, for example, digestion using any number of known proteases to release peptides. The role of the peptides in specific binding to gp120 protein of HIV-1 can be confirmed using the assays as described herein in the Experimental Examples.

In another embodiment, the peptides of the invention may be prepared using a biochemical synthesis method. Biochemical methods for synthesizing peptides are well known to those skilled in the art. The peptides may also be produced by recombinant means using procedures known to the skilled artisan and described herein in the Experimental Examples.

In a preferred embodiment, the peptides comprising the anti-viral compositions of the invention are capable of binding to the stem of the V3 loop of the viral envelope protein, gp120, from an HIV-1 virion. The ability to bind to gp120 protein from an HIV-1 virion may be assessed using assays described herein. Also described are the methods for demonstrating the ability of these peptides of preventing HIV infectivity. The peptides of the invention may be administered to a patient to inhibit HIV-1 infectivity or to prevent infection by HIV-1. The inhibition of infectivity of HIV-1 by the peptides of the invention may be assessed as described herein. Such methods may include p24 assay, reverse transcriptase activity assay or TCID₅₀.

The invention also provides for an antibody which is capable of specifically binding to the peptides of the invention, in particular, those having the amino acid sequences as set forth in SEQ ID NOS: 1, 2, 3, 4 and 7. The antibody of the invention may be a monoclonal or a polyclonal antibody, or may be a synthetic, humanized or phage displayed antibody. The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The invention also includes a kit for detecting a peptide which inhibits the infectivity of HIV-1. The peptides include those designated as SEQ ID NOS: 1, 2, 3, 4 and 7. The kit of the invention, may, for example, be an ELISA kit which includes an antibody, a detection reagent, and a reaction surface. In one embodiment, the antibody is an antibody of the invention which specifically binds with any one of the peptides described herein or derivatives, or fragments thereof. The antibody may be any type of antibody described herein and may be made using any of the methods described herein. The reaction surface may be a microtiter plate, such as an ELISA plate. The detection reagent may be any detection reagent known to those skilled in the art. For example, the detection reagent may be an enzyme, or a radionuclide. In one embodiment, the kit of the invention is an ELISA kit for detecting the presence of any one of SEQ ID NOS: 1, 2, 3, 4 and 7 in a bodily fluid such as cervical vaginal lavage, semen or saliva of a human patient.

The kit may include a microwell plate, an antibody which is capable of specifically binding any one or more of SEQ ID NOS: 1, 2, 3, 4, and 7 and a secondary enzyme capable of binding the antibody of the invention and also horseradish peroxidase. The ELISA kit of the invention may be used, for example, to carry out an ELISA assay of a bodily fluid of a patient, such as a saliva sample. The assay may be used to detect and quantify levels of these peptides present in the saliva of the patient. The quantity of any one of these peptides in the patient's saliva may be correlated with the ability of the patient's saliva to inhibit the infectivity of HIV-1.

In another embodiment, the kit of the invention is a Western Blotting or dot blotting kit for detecting the presence of any one of the peptides set forth in SEQ ID NOS: 1, 2, 3, 4, and 7 in a bodily fluid such as semen, cervical vaginal lavage or saliva of a human patient.

The kits of the present invention may be used, for example, to assess the susceptibility of a patient to HIV-1 infection. Patients with high susceptibility to HIV-1 infection due to low levels of these peptides may be treated with one of the pharmaceutical compositions of the invention to enhance resistance of these individuals to HIV-1 infection. The correlation between the levels of these peptides with the ability of a patient to inhibit the infectivity of HIV-1 is established using the procedures described in the Experimental Examples presented herein.

The invention also includes a method of inhibiting the infectivity of HIV-1 in bodily fluids, such as, but not limited to cervical vaginal lavage, semen, or in infective oral secretions. The method is useful in preventing HIV-1 infection, or inhibiting the infectivity of HIV-1. This method can be used, for example to inhibit the infectivity of oral secretions, for example in a hospital setting where medical personnel are exposed to infectious HIV-1 secretions.

In one embodiment, the method comprises contacting an HIV-1 virion with the peptide compositions described herein. In one embodiment, the composition may comprise substantially purified peptides. The sample from a patient containing the HIV-1 virion may be obtained from any sample of bodily fluid, such as cervical vaginal lavage (CVL), semen or a saliva sample, or a blood sample. In one embodiment, a composition comprising substantially purified peptides of the invention is contacted with an HIV-1 virion from a sample of a patient for a period of time sufficient for the peptides to inhibit the infectivity of HIV-1. The inhibition of the infectivity of HIV-1 can be assessed as described herein in the Examples.

In another embodiment, the method of inhibiting the infectivity of HIV-1 comprises contacting an HIV-1 virion obtained from a bodily fluid sample of a patient, such as cervical vaginal lavage, semen, or saliva with a composition having a surface which contains a substantially purified peptide associated with said surface. The surface may be a condom, a diaphragm, a vaginal ring or a suppository. Other examples of such surfaces include plastic or other polymer surfaces, which are inert to reaction with bodily fluids, and are considered biocompatible. In one embodiment of the method of the invention, the composition having substantially purified peptides of the invention associated with the surface is contacted with a body fluid of a patient or an infective oral secretion which contains an HIV-1 virion. The composition is contacted or incubated with the sample of bodily fluid containing the HIV-1 virion for a period of time sufficient to inhibit the infectivity of HIV-1. The inhibition of the infectivity of HIV-1 can be assessed by assays as described herein in the Experimental Examples section. In another embodiment, the peptides of the invention may be used as described herein to aid in the cleansing of blood or blood products in the event the blood or blood products are suspected of being contaminated with HIV. Alternatively, the peptides of the invention may be used in plasmapheresis procedures to aid in the elimination of HIV particles from a patient having or suspected of having an HIV infection.

In another embodiment, methods are provided for stabilizing the peptides of the invention by attachment to scaffolds or polymers or by preparing them as a dendrimeric complex, as described below. These methods for stabilization may aid in prevention of proteolytic breakdown and thus an increase in the half-life of the peptides, derivatives, fragments or mimics thereof.

Methods for screening for novel anti-HIV peptides are also provided using the methods described herein.

Peptides of the Invention for use in Inhibiting Retrovirus Infectivity and Methods for Modifying the Peptides of the Invention

The present invention provides for the identification of several peptides obtained from salivary agglutinin or gp340 that have the capacity of binding to HIV and inhibiting HIV infectivity. More particularly, these peptides, which have the amino acid sequences as set forth in SEQ ID NOS: 1, 2, 3 and 4 bind to the stem of the V3 loop of the gp120 envelope protein of HIV, thereby inhibiting the infectivity of the virus. These peptides, or derivatives (analogs, variants, mimics and active fragments thereof) may be prepared using the well known techniques of solid phase, liquid phase, or peptide condensation techniques, or any combination thereof. These peptides may also be prepared using recombinant means. Moreover, these peptides may retain their binding to HIV and anti-HIV activity if conservative amino acid substitutions are made. These peptides may be formulated as pharmaceutical compositions for prophylactic or therapeutic use. These peptide compositions may be effective when delivered orally or parenterally, or as a topical formulation or for delivery to a mucosal surface.

Several strategies have been pursued in efforts to increase the effectiveness of antimicrobial peptides (Tam, J. P.; Lu, Y. A,; Yang, J. L. Antimicrobial dendrimeric peptides. Eur. J. Biochem. 2002, 269 (3), 923-932; Janiszewska, J.; Swieton, J.; Lipkowski, A. W.; Urbanczyk-Lipkowska, Z. Low molecular mass peptide dendrimers that express antimicrobial properties. Bioorg. Med. Chem. Lett. 2003, 13 (21). 3711-3713; Tam et al. Eur. J. Biochem. (2002), February: 269(3):923-932; Dathe et al., Biochem. (2004), July 20; 43(28):9140-9150; Tang et al., (1999), Science, Oct. 15; 286 (5439):498-502; Dempsey et al, Biochem., (2003), January 21; 42(2):402-409; Epand et al., Biochem., (2004), July 27; 43(29):9527-9535; Papo et al., Biochem., (2004), June; 43(21):6393-6403). Sequence changes in natural peptides can notably reduce hemolysis while preserving activity (Staubitz, P., Peschel, A, Nieuwenhuizen, W. F., Otto, M., Gotz, F., Jung, G. and Jack, R W. (2001). Structure-function relationships in the tryptophan-rich, antimicrobial peptide indolicidin. J Pept Sci 7(10): 552-564). Inserting unnatural D-amino acids or beta-amino acids into peptide sequences, combinatorial designs based on linear or cyclic sequences (Houghton, Ghadiri), synthetic chemical mimetics (DeGrado, Tew), and multivalent dendrimeric constructs of short peptides (Janiszewska, J.; Swieton, J.; Lipkowski, A. W.; Urbanczyk-Lipkowska, Z. Low molecular mass peptide dendrimers that express antimicrobial properties. Bioorg. Med. Chem. Lett. 2003, 13 (21). 3711-3713; Xing, 2003) are other alternatives. In some cases improved solubility, salt resistance, stability and toxicity have been reported, with some reduction in IC₅₀ (Tam, 2002).

Accordingly, many different designs for therapeutics have been reported, seeking to develop or improve activity under physiological conditions, low toxicity and proteolytic stability. Among promising approaches, polyvalent or multivalent antimicrobial polymers offers promise for enhancing the efficacy of existing antimicrobial monomer peptide and minimizing the problems accompanying conventional antimicrobial peptides by reducing the toxicity of the residue, increasing their efficiency and selectivity, and prolonging the lifetime of the effect. Especially, these include their ability to amplify cationic charges and hydrophobic clusters as the number of monomer increases. (Tam, J. P.; Lu, Y. A,; Yang, J. L. Antimicrobial dendrimeic peptides. Eur. J. Biochem. 2002, 269 (3), 923-932). For example, the multivalency of peptides incorporated with fragments of known antibacterial peptides in dendrimers has appeared to demonstrate good activity in the design of membranolytic peptides for therapeutic applications (Tam, J. P.; Lu, Y. A,; Yang, J. L. Antimicrobial dendrimeic peptides. Eur. J. Biochern. 2002, 269 (3), 923-932).

In this connection, U.S. Pat. No. 5,229,490 to Tam discloses a particular polymeric construction formed by the binding of multiple antigens to a dendritic core or backbone, the objective of which is to potentiate the concentration of antigen within a more economical and efficient molecule. While this construction has demonstrated advantages, greater activity and corresponding stability of the construct is still an important objective that is not fulfilled therein.

U.S. Pat. No. 3,679,653 to Schuck et al. discloses the preparation and use of polymer-based protein complexes, and particularly, relates to the preparation of such complexes with hormones such as bovine growth hormone, insulin and the like. Schuck et al. however, prepare complexes with full length native hormones, and bind the native material to the polymer backbone for the purpose of improving the delivery and availability of such hormones. The inventors qualify that the level of activity of the resulting complexes are somewhat uncertain, and in any event, do not represent that any dramatic improvements in such activity are either anticipated or realized.

It is envisioned that the active anti-HIV peptides of the invention, while having primarily natural amino acids, may also be synthesized to include unnatural or unusual synthetic amino acids.

Amino acids used for peptide synthesis may be standard Boc (N-α.-amino protected N-α.-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile N-α.-amino protected 9-fluorenylmethoxycarbonyl (Fmoc) amino acids first described by Carpino and Han (1972, J. Org. Chem. 37:3403-3409).

Thus, polypeptides of the invention may comprise D-amino acids, a combination of D- and L-amino acids, and various “designer” amino acids (e.g., P-methyl amino acids, C-α-methyl amino acids, and N-α-methyl amino acids, etc.) to convey special properties.

Synthetic amino acids that may be used include ornithine for lysine, fluorophenylalanine for phenylalanine, and norleucine for leucine or isoleucine. Additionally, by assigning specific amino acids at specific coupling steps, α-helices, β turns, β sheets, γ-turns, and cyclic peptides can be generated.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

In one aspect of the invention, the peptides may comprise a special amino acid at the C-terminus which incorporates either a CO₂H or CONH₂ side chain to simulate a free glycine or a glycine-amide group. Another way to consider this special residue would be as a D or L amino acid analog with a side chain consisting of the linker or bond to the bead. In one embodiment, the pseudo-free C-terminal residue may be of the D or the L optical configuration; in another embodiment, a racemic mixture of D and L-isomers may be used.

Alternatively, pyroglutamate may be included as the N-terminal residue of the peptide. Although pyroglutamate is not amenable to sequence by Edman degradation, by limiting substitution to only 50% of the peptides on a given bead with N-terminal pyroglutamate, there will remain enough non-pyroglutamate peptide on the bead for sequencing. One of ordinary skill would readily recognize that this technique could be used for sequencing of any peptide that incorporates a residue resistant to Edman degradation at the N-terminus. Other methods to characterize individual peptides that demonstrate desired activity are described in detail infra. Specific activity of a peptide that comprises a blocked N-terminal group, e.g., pyroglutamate, when the particular N-terminal group is present in 50% of the peptides, would readily be demonstrated by comparing activity of a completely (100%) blocked peptide with a non-blocked (0%) peptide.

In addition, the present invention envisions preparing peptides that have more well defined structural properties, and the use of ester bonds, to prepare peptides with novel properties. In another embodiment, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a molecule would be resistant to peptide bond hydrolysis, e.g., protease activity. Such peptides would provide ligands with unique function and activity, such as extended half-lives in vivo due to resistance to metabolic breakdown, or protease activity. Furthermore, it is well known that in certain systems constrained peptides show enhanced functional activity (Hruby, 1982, Life Sciences 31:189-199; Hruby et al., 1990, Biochem J. 268:249-262).

A constrained, cyclic or rigidized peptide may be prepared synthetically, provided that in at least two positions in the sequence of the peptide, an amino acid or amino acid analog is inserted that provides a chemical functional group capable of cross-linking to constrain, cyclize or rigidize the peptide after treatment to form the cross-link. Cyclization will be favored when a turn-inducing amino acid is incorporated. Examples of amino acids capable of cross-linking a peptide are cysteine to form disulfide, aspartic acid to form a lactone or a lactase, and a chelator such as γ-carboxyl-glutamic acid (Gla) (Bachem) to chelate a transition metal and form a cross-link. Protected γ-carboxyl glutamic acid may be prepared by modifying the synthesis described by Zee-Cheng and Olson (1980, Biophys. Biochem. Res. Commun. 94:1128-1132). A peptide in which the peptide sequence comprises at least two amino acids capable of cross-linking may be treated, e.g., by oxidation of cysteine residues to form a disulfide or addition of a metal ion to form a chelate, so as to cross-link the peptide and form a constrained, cyclic or rigidized peptide.

The present invention provides strategies to systematically prepare cross-links. For example, if four cysteine residues are incorporated in the peptide sequence, different protecting groups may be used (Hiskey, 1981, in The Peptides: Analysis, Synthesis, Biology, Vol. 3, Gross, and Meienhofer, eds., Academic Press: New York, pp. 137-167; Ponsanti et al., 1990, Tetrahedron 46:8255-8266). The first pair of cysteine may be deprotected and oxidized, then the second set may be deprotected and oxidized. In this way a defined set of disulfide cross-links may be formed. Alternatively, a pair of cysteine and a pair of collating amino acid analogs may be incorporated so that the cross-links are of a different chemical nature.

The following non-classical amino acids may be incorporated in the peptide in order to introduce particular conformational motifs: 1,2,3,4-tetrahydroisoquinoline-3-carboxylate (Kazmierski et al., 1991, J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby, 1991, Tetrahedron Lett.); 2-aminotetrahydronaphthalene-2-carboxylic acid (Landis, 1989, Ph.D. Thesis, University of Arizona); hydroxy-1,2,3,4-tetrahydroisoquinoline-3 carboxylate (Miyake et al., 1989, J. Takeda Res. Labs. 43:53-76); β-carboline (D and L) (Kazmierski, 1988, Ph.D. Thesis, University of Arizona); HIC (histidine isoquinoline carboxylic acid) (Zechel et al., 1991, Int. J. Pep. Protein Res. 43); and HIC (histidine cyclic urea) (Dharanipragada).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al., 1985, J. Org. Chem. 50:5834-5838); β-sheet inducing analogs (Kemp et al., 1988, Tetrahedron Lett. 29:5081-5082); β turn inducing analogs (Kemp et al., 1988, Tetrahedron Lett. 29:5057-5060); various-helix inducing analogs (Kemp et al., 1988, Tetrahedron Lett. 29:4935-4938), γ-turn inducing analogs (Kemp et al., 1989, J. Org. Chem. 54:109:115); and analogs provided by the following references: Nagai and Sato, 1985, Tetrahedron Lett. 26:647-650; DiMaio et al., 1989, J. Chem. Soc. Perkin Trans. p. 1687; also a Gly-Ala turn analog (Kahn et al., 1989, Tetrahedron Lett. 36:2317); amide bond isostere (Jones et al., 1988, Tetrahedron Lett. 29:3853-3856); tretrazol (Zabrocki et al., 1988, J. Am. Chem. Soc. 110:5875-5880); DTC (Samanen et al., 1990, Int. J. Protein Pep. Res. 35.:501-509), and analogs taught in Olson et al., 1990, J. Am. Chem. Sci. 112:323-333 and Garvey et al., 1990, J. Org. Chem. 56:436. Conformationally restricted mimetics of beta turns and beta bulges, and peptides containing them, are described in U.S. Pat. No. 5,440,013, issued Aug. 8, 1995 to Kahn.

The present invention further provides for modification or derivatization of the polypeptide or peptides of the invention. Modifications of peptides are well known to one of ordinary skill, and include phosphorylation, carboxymethylation, and acylation. Modifications may be effected by chemical or enzymatic means.

In another aspect, glycosylated or fatty acylated peptide derivatives may be prepared. Preparation of glycosylated or fatty acylated peptides is well known in the art. Fatty acyl peptide derivatives may also be prepared. For example, and not by way of limitation, a free amino group (N-terminal or lysyl) may be acylated, e.g., myristoylated. In another embodiment an amino acid comprising an aliphatic side chain of the structure —(CH₂)ₙCH₃ may be incorporated in the peptide. This and other peptide-fatty acid conjugates suitable for use in the present invention are disclosed in U.K. Patent GB-8809162.4, International Patent Application PCT/AU89/00166, and reference 5, supra.

Chemical Moieties for Derivatization. Derivatives of the peptides (including variants, analogs, mimics and active fragments thereof) of the present invention are further provided. Such derivatives encompass and include derivatives to enhance activity, solubility, effective therapeutic concentration, and transport across the blood brain barrier. Further encompassed derivatives include the attachment of moieties or molecules which are known to target the peptides or derivatives thereof to a cell that is susceptible to HIV infection. The chemical moieties may be N-terminally or C-terminally attached to the peptides of the present invention. Chemical moieties suitable for derivatization may be, for instance, selected from among water soluble polymers. The polymer selected can be water soluble so that the component to which it is attached does not precipitate in an aqueous environment, such as a physiological environment. Preferably, for therapeutic use of the end-product preparation, the polymer will be pharmaceutically acceptable. The polymer may be branched or unbranched. One skilled in the art will be able to select the desired polymer based on such considerations as whether the polymer/component conjugate will be used therapeutically, and if so, the desired dosage, circulation time, resistance to proteolysis, and other considerations. For the present component or components, these may be ascertained using the assays provided herein.

The water soluble polymer may be selected from the group consisting of, for example, polyethylene glycol, copolymers of ethylene glycoupropylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols and polyvinyl alcohol. Polyethylene glycol propionaldehyde may have advantages in manufacturing due to its stability in water.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The number of polymer molecules so attached may vary, and one skilled in the art will be able to ascertain the effect on function. One may mono-derivative, or may provide for a di-, tri-, tetra- or some combination of derivatization, with the same or different chemical moieties (e.g., polymers, such as different weights of polyethylene glycols). The proportion of polymer molecules to component or components molecules will vary, as will their concentrations in the reaction mixture. In general, the optimum ratio (in terms of efficiency of reaction in that there is no excess unreacted component or components and polymer) will be determined by factors such as the desired degree of derivatization (e.g., mono, di-, tri-, etc.), the molecular weight of the polymer selected, whether the polymer is branched or unbranched, and the reaction conditions.

The polyethylene glycol molecules (or other chemical moieties) should be attached to the component or components with consideration of effects on functional or antigenic domains of the protein. There are a number of attachment methods available to those skilled in the art, e.g., EP 0 401 384 herein incorporated by reference (coupling PEG to G-CSF), see also Malik et al., 1992, Exp. Hematol. 20:1028-1035 (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group include lysine residues and the—terminal amino acid residues; those having a free carboxyl group include aspartic acid residues glutamic acid residues and the C-terminal amino acid residue. Sulfhydryl groups may also be used as a reactive group for attaching the polyethylene glycol molecule(s). Preferred for therapeutic purposes is attachment at an amino group, such as attachment at the N-terminus or lysine group.

The invention also provides derivatives wherein at least one of said attached chemical moieties is a molecule which facilitates transfer or transport across the blood-brain barrier, particularly molecules that naturally cross the blood-brain barrier. This is especially useful when one is contemplating use of the peptides or proteins of the invention for use as an anti-HIV agent. Thus, the compositions containing the proteins or peptides of the invention that are formulated for enhancing transport across the blood brain barrier may prove useful for treating dementia associated with AIDS and HIV infectivity. Examples of such transport enhancing molecules include a biocompatible hydrophobic molecule, transferrin or apolipoprotein. Transferrin has been shown to facilitate transfer, even or larger peptides, as for example, nerve growth factor (Friden, P. M. et al., Science 259, 373-377 (1993), Kordower, J. H. et al., Proc Natl Acad Sci USA 91, 9077-9080 (1994)). Apolipoprotein E (ApoE) and apolipoproetin J (ApoJ) have been shown to facilitate brain uptake of Alzheimer's amyloid beta protein when complexed thereto (Zlokovic, B. V. et al., Biochem. Biophys. Res. Commun. 205 (2), 1431-1437 (1994); Martel, C. L. et al., J. Neurochem 69(5), 1995-2004 (1997)).

The present invention also provides derivatives which are fusion proteins comprising the peptides of the present invention or fragments thereof. Thus peptides of the present invention and fragments thereof can be “modified” i.e., placed in a fusion of chimeric peptide or protein, or labeled, e.g., to have an N-terminal FLAG-tag. In a particular embodiment a peptide can be modified by linkage or attachment to a marker protein such as green fluorescent protein as described in U.S. Pat. No. 5,625,048 filed Apr. 29, 1997 and WO 97/26333, published Jul. 24, 1999 (each of which are hereby incorporated by reference herein in their entireties).

In one such embodiment, a chimeric peptide can be prepared, e.g., a glutathione-S-transferase (GST) fusion protein, a maltose-binding (MPB) protein fusion protein, or a poly-histidine-tagged fusion protein, for expression in a eukaryotic cell. Expression of the peptide of the present invention as a fusion protein can facilitate stable expression, or allow for purification based on the properties of the fusion partner. For example, GST binds glutathione conjugated to a solid support matrix, MBP binds to a maltose matrix, and poly-histidine chelates to a Ni-chelation support matrix. The fusion protein can be eluted from the specific matrix with appropriate buffers, or by treating with a protease specific for a cleavage site usually engineered between the peptide and the fusion partner (e.g., GST, MBP, or poly-His). Alternatively the chimeric peptide may contain the green fluorescent protein, and be used to determine the intracellular localization of the peptide in the cell.

The invention also includes derivatives wherein at least one of the attached chemical moieties is a molecule having multiple sites for peptide attachment and capable of binding at least two of said peptides simultaneously to generate a multimeric peptide structure. This derivative has the effect of increasing the available local concentration of the peptide(s) of the present invention. Alternatively, or in addition, such moieties can function in providing a stable scaffold to retain the peptide in place for activity, thereby reducing or preventing diffusion or degradation. Such molecule may be selected from the group of BSA, ovalbumin, human serum albumin, polyacrylamide, beads and synthetic fibers (biodegradable and non-biodegradable).

Peptide Monomers, Dimers and Multimers

The peptides of the present invention that exhibit the anti-HIV activity may be prepared and utilized as monomers, dimers, multimers, heterodimers, heteromultimers, etc, as described herein. Presentation or administration of the peptide in multimeric form may result in enhanced activity, longer half-life or otherwise increased modulation of the activity mediated by the relevant amino acid residues, including the interaction of these relevant residues with the HIV gp120 envelope.

Monomers

The peptide monomer could be produced in a variety of ways, including the methods described herein. The peptide of the present invention can also be synthesized using a protein synthesizer and utilizing methods well known in the art and as described hereinabove, incorporating amino acid modifications, analogs, etc. as hereinabove described. In addition, the DNA sequence encoding the peptide can be inserted into an expression vector such as pSE (Invitrogen) or pcDNA3 (Invitrogen) for production in bacterial or mammalian cell expression systems. Insect or yeast expression systems could also be used. Purification of the peptide could be facilitated by the addition of a tag sequence such as the 6-Histidine tag which binds to Nickel-NTA resins. These tag sequences are often easily removed by the addition of a protease specific sequence following the tag. Dimers, Multimers

Dimers and multimers of the anti-HIV peptide can be produced using a variety of methods in the art. The DNA sequence of a dimer or multimer could also be inserted into an expression system such as bacteria or mammalian cell systems. This could produce molecules such as Met-FLHTRLFV)ₓ where x=2, 3, 4, . . . etc. It may be necessary to include a short flexible spacer (Gly-Gly-Gly-Gly-Ser)₃ between the peptidomimetic to increase its effectiveness.

Dimers and multimers can also be generated using crosslinking reagents such as Disuccinimidyl suberate (DS S) or Dithiobis (succinimidyl propionate) (DSP). These reagents are reactive with amino groups and could crosslink the carbohydrate epitope mimic peptide through free amine groups at the arginine residues and the free amine group at the N-terminus.

Dimers and multimers can also be formed using affinity interactions between biotin and avidin, Jun and Fos, and the Fc region of antibodies. The purified peptide can be biotinylated and mixed with factors that are known to form strong protein-protein interactions. The peptide could be linked to the regions in Jun and Fos responsible for dimer formation using crosslinkers such as those mentioned above or using molecular techniques to create a peptide-Jun/Fos molecule. When the Jun and Fos peptide hybrids are mixed, dimer formation would result. In addition, production of a peptide-Fc hybrid could also be produced. When expressed in mammalian cells, covalent disulfide bonds form through cysteines in the Fc region and dimer formation would result.

Heterodimers, Heteromultimers

Heterodimers and heteromultimers of the anti-HIV peptide could also be produced. The same technologies as those listed above could be used to generate these molecules. Molecular techniques could be used to insert the peptide into a protein at the DNA level. This insertion could take place at the N- or C-terminus, or in the middle of the protein molecule. Heterodimers could be formed using peptide/Fc or peptide/Jun or Fos hybrid molecules. When mixed with other Fc or Jun/Fos containing hybrids, dimer formation would result producing heterodimers. Crosslinking reagents could also be used to link the peptide to heterodimers. Lastly, biotinylation of the peptide along with biotinylation of other molecules could be used to create multimers. Mixing of these components with avidin could create large multifunctional complexes, where each of the four biotin binding sites of the avidin molecule is occupied by a different biotinylated molecule.

Linear Polymeric Complexes

The polymer-peptide monomer complexes comprise antimicrobial/antiviral/antifungal peptide monomers covalently bound to a polymer backbone. The peptide component of the complex may be and include any sequence of amino acids that has a lethal effect on bacteria, viruses or fungi in its monomeric form. More particularly, the peptides may be any antimicrobial peptides, including natural products found in organisms, fragments of natural peptides, and any synthetic analogs or de novo designs. These peptides can accordingly include nonnatural amino acids: beta-amino acids, d-amino acids and/or non-indigenous amino acids.

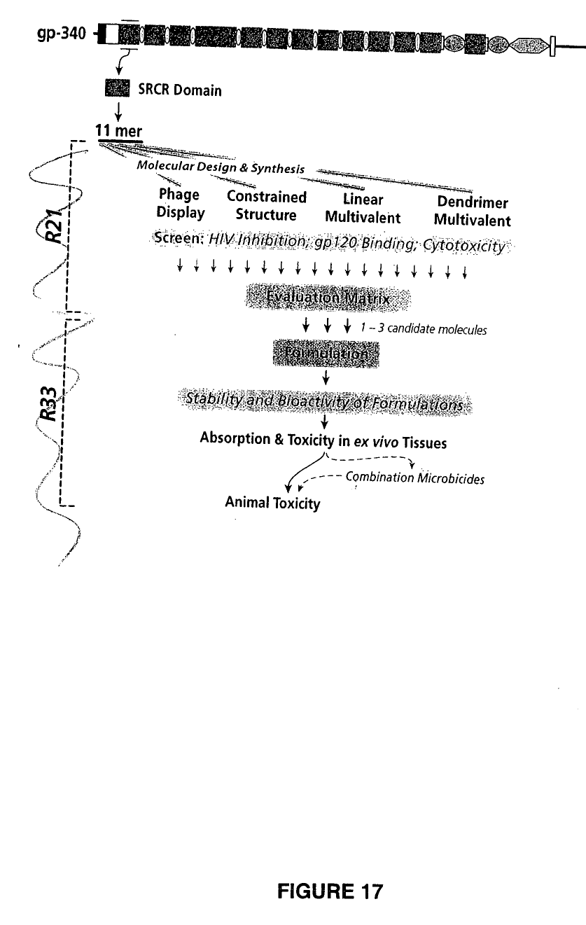

While numerous antimicrobial and antiviral peptides have been identified and are known, the present invention relates to the gp340 protein and the anti-HIV properties associated with several polypeptides derived from the parental gp340 molecule, more particularly, the polypeptides having the amino acid sequences as set forth in SEQ ID NOS: 1, 2, 3 and 4. Furthermore, it is reasonable to assume that the procedures provided for identifying novel polypeptides or mimics thereof will also result in the identification of polypeptides that may have broader anti-microbial activity, e.g. anti-bacterial or anti-fungal activity, not only anti-viral activity. In yet another particular embodiment, the use of reactive linear polymers to amplify the valency of such molecules is a practical alternative. An overview of the interplay between the various aspects of the invention is shown in FIG. 17.

Suitable polymers may include linear polymers or copolymers such as those described in greater detail hereinafter, or nonlinear structures, such as dendrimers. Any molecule with multiple reactive sites can be a suitable scaffold for the peptide monomers that are a part of the complexes of the invention. In a particular embodiment, the polymers may preferably contain carboxyl or anhydride linkages, especially where the peptide contains an amino, hydroxyl (including phenolic hydroxyl), or sulfhydryl group not essential for its activity. Where the peptide contains a carboxyl group not essential for activity, the polymer can contain free hydroxyl or amine groups for reaction, therewith. In one non-limiting example, the polymer is PMA or EMA, but it can be any of those types disclosed for coupling or reaction with a peptide, and in any event it is adapted to effect covalent bonding with the peptide to produce a peptide-polymer product either directly or indirectly using an activating agent. In another non-limiting example, the polymer is a biocompatible polymer and is selected from a polymaleic anhydride and poly (ethylene/maleic anhydride) copolymer, and wherein the biocompatible polymer has a molecular weight in the range of about 1,000 to about 1,000,000; and a plurality of monomeric peptides that inhibit bacterial, viral or fungal infectivity covalently bound thereto. Inasmuch as the antimicrobial/antiviral/antifungal activity of the starting peptide is desired to be not only retained but significantly increased in the final product by several orders of magnitude over the peptide alone, it is, of course, necessary that bonding of the peptide to the polymer be through a group which will not result in inactivation of an active site on the peptide. Among the various reactive groups of peptides may be mentioned, besides amino and sulfhydryl, also hydroxyl (including phenolic hydroxyl), carboxyl and imidazolyl. Such groups are present in free or unbound form in inactive portions of peptide molecules, as in a lysine, cysteine, serine, aspartic acid, glutamic acid, threonine, histidine, or tyrosine moiety, where the particular moiety in question is not considered essential for activity. Therefore, attachment to the polymer molecule is through reaction of the polymer with such groups so as to avoid inactivation of the peptide during attachment to the polymer molecule.

Generally, the linkage is an amide, imide, ester, thioester, or disulfide group, such as formed by the carboxyl or anhydride of the polymer with an amine or hydroxyl group in a non-essential moiety of the peptide chain. Amides are formed by reacting pendant amino groups of the peptide with carboxylic anhydride groups of the carrier polymer in water, in aqueous buffer media, or in mixed solvents. Amides, imides and esters are readily formed by activating carboxyl groups of the polymer, or alternatively pendant carboxyls of the peptide, and reacting them with respective hydroxyl, amine or mercaptan groups on the other reactant. Such activation may be effected using various carbodiimides, carbodiimidazoles, Woodward's or Sheehan's reagent, or the like, to form highly active intermediates capable of reacting with other groups mentioned above under mild conditions, the latter favoring retention of relevant peptidic activity.

The polymer selected for such reaction can therefore be said to be adapted to couple or react with the peptide, either directly or indirectly through use of an activating agent, as already indicated, and in any event to effect covalent bonding with the peptide. The attachment procedures given are conducted by techniques adapted to include any requisite protection for the peptide, which may include a reversible blocking of any active site or sites.

In its broadest context, the polymer to which the peptides are coupled contains carboxyl or anhydride linkages, especially where the peptides contain an amino, hydroxyl, or sulfhydryl group not essential for their activity. Where a peptide contains a carboxyl group not essential for activity, the polymer can contain hydroxyl or amine groups for reaction herewith. Exemplary polymers include PMA or EMA, or may be any of those types previously disclosed for coupling or reaction with the peptide component, and in any event it is adapted to couple or react with the peptides to effect covalent bonding and production of the desired monomeric peptide-polymer complex or product.

Since covalent bonding is desired, it is understood that the carrier polymer is tailored to contain at least one reactive site for each polymer molecule with which the peptides can react, either directly or indirectly, to produce a covalent bond. According to the instant invention, this reactive site (or sites) is preferably a carboxyl or carboxylic anhydride group.

In a non-limiting example, the polymeric reactant is a polymer (a) comprising chains of carboxylic acid or carboxylic acid anhydride units, or (b) comprising units of carboxylic acid or carboxylic acid anhydride groups separated by carbon chains of at least one and not more than four carbon atoms, said carbon chains being part of a unit which contains a maximum of eighteen carbon atoms, said polymers being formed by polymerization of polymerizable acids or anhydrides or by copolymerizing a polymerizable acid or anhydride with another copolymerizable monomer, and preferably wherein the starting acid or anhydride and any additional copolymerizable monomer are unsaturated and such polymerization or copolymerization comprises addition type polymerization or copolymerization involving such unsaturation.

Many of these polymers are commercially available and others are simple derivatives of commercially available products, which can be readily prepared either prior to or simultaneously with the hormone attachment reaction, or produced as a minor modification of the basic polymer after attachment. Such polymers containing the above-described EMA-type units are described above and are hereinafter referred to as an “EMA-type polymer.”

A particular type of polymeric material useful in the practice of the invention is the polymer of an olefinically unsaturated polycarboxylic acid or derivative with itself or in approximately equimolar proportions with at least one other monomer copolymerizable therewith. The polycarboxylic acid derivative can be of the non-vicinal type, including acrylic acid, acrylic anhydride, methacrylic acid, crotonic acid or their respective derivatives, including partial salts, amides and esters or of the vicinal type, including maleic, itaconic, citraconic, α,α-dimethyl maleic, α-butyl maleic, α-phenyl maleic, fumaric, aconitic, α-chloromaleic, α-bromomaleic, α-cyanomaleic acids including their partial salts, amides and esters. Anhydrides of any of the foregoing acids are advantageously employed.

Co-monomers suitable for use with the above functional monomers include α-olefins such as ethylene, propylene, isobutylene, 1- or 2-butene, 1-hexene, 1-octene, 1-decene, 1-dodecene, 1-octadecene, and other vinyl monomers such as styrene, α-methyl styrene, vinyl toluene, vinyl acetate, vinyl amine, vinyl chloride, vinyl formate, vinyl propionate, vinyl alkyl ethers, e.g., methylvinylether, alkyl acrylate, alkyl methacrylates, acrylamides and alkylacryl amides, or mixtures of these monomers. Reactivity of some functional groups in the copolymers resulting from some of these monomers permits formation of other useful functional groups in the formed copolymer including hydroxy, lactone, amine and lactam groups.

Any of the polybasic acid derivatives may be copolymerized with any of the other monomers described above, and any other monomer which forms a copolymer with dibasic acid derivatives. As an additional modification, the polybasic acid derivatives can be copolymers with a plurality of comonomers, in which case the total amount of the comonomers will preferably be about equimolar with respect to the polybasic acid derivatives. Although these copolymers can be prepared by direct polymerization of the various monomers, frequently they are more easily prepared by an after-reaction modification of an existing copolymer.

Copolymers of anhydrides and another monomer can be converted to carboxyl-containing copolymers by reaction with water, and to ammonium, alkali and alkaline earth metal and alkylamine salts thereof by reaction with alkali metal compounds, alkaline earth metal compounds, amines or ammonia, either prior to, during, or subsequent to peptide attachment, etc. Other suitable derivatives of the above polymers include the partial alkyl or other esters and partial amides, alkyl amides, dialkyl amides, phenyl alkyl amides or phenyl amides prepared by reacting carboxyl groups on the polymer chain with the selected amines or alkyl or phenylalkyl alcohol as well as amino esters, amino amides, hydroxy amides and hydroxy esters, wherein the functional groups are separated by lower alkylene, phenyl, phenylalkyl, phenylalkylphenyl, or alkylphenylalkyl, which are prepared in the same manner in each case with due consideration of preservation of peptide attachment sites as previously stated. Other aryl groups may be present in place of phenyl groups. Particularly useful derivatives are those in which negatively charged carboxyl groups are partially replaced with amine or amine salt groups. These are formed by reaction of said carboxyls with polyamines such as dimethylamino propylamine or dialkylaminoalcohols such as dimethylaminoethanol, the former forming an amide linkage with the polymer and the latter an ester linkage. Suitable selection of the above derivatives permits control of several parameters of performance for the peptide-polymer complexes of the invention.

Representative dibasic acid or anhydride-olefin polymers, especially maleic acid or anhydride-olefin polymers, of the foregoing type (EMA-type) are known, for example, from U.S. Pat. Nos. 2,378,629, 2,396,785, 3,157,595 and 3,340,680. Generally, the copolymers are prepared by reacting ethylene or other unsaturated monomer or mixtures thereof, as previously described, with the acid anhydride in the presence of a peroxide catalyst in an aliphatic or aromatic hydrocarbon solvent for the monomers but nonsolvent for the interpolymer formed. Suitable solvents include benzene, toluene, xylene, chlorinated benzene and the like. While benzoyl peroxide is usually the preferred catalyst, other peroxides such as acetyl peroxide, butyryl peroxide, di-tertiary butyl peroxide, lauroyl peroxide and the like, or any of the numerous azo catalysts, are satisfactory since they are soluble in organic solvents. The copolymer preferably contains substantially equimolar quantities of the olefin residue and the anhydride residue. Generally, it will have a degree of polymerization of 8 to 10,000, preferably about 100 to 5,000, and a molecular weight of about 1,000 to 1,000,000, preferably about 10,000 to 500,000. The properties of the polymer, such as molecular weight, for example, are regulated by proper choice of the catalyst and control of one or more of the variables such as ratio of reactants, temperature, and catalyst concentration or the addition of regulating chain transfer agents, such as diisopropyl benzene; propionic acid, alkyl aldehydes, or the like. The product is obtained in solid form and is recovered by filtration, centrifugation or the like. Removal of any residual or adherent solvent can be effected by evaporation using moderate heating. Numerous of these polymers are commercially available. Particularly valuable copolymers are homopolymers of maleic anhydride (PMA), and those derived from ethylene and maleic anhydride (PEMA) in approximately equimolar proportions. Both products are commercially available.

The maleic anhydride homopolymers and copolymers thus obtained have repeating anhydride linkages in the molecule, which are readily hydrolyzed by water to yield the acid form of the copolymer, rate of hydrolysis being proportional to temperature. In view of the fact that the attachment or coupling reactions of the present invention are carried out in aqueous solutions or suspensions, or using water-solvent mixtures, the product of the covalent bonding of the hormone to PMA or PEMA has carboxyl or carboxylate groups attached to its chains adjacent the attached hormone in stead of anhydride groups, due to hydrolysis of the anhydride groups, which do not react with the hormone, during the reaction. The same is true of nonreacting anhydride groups present in other polymers, such as EMA type polymers, which hydrolyze to carboxyl or carboxylate groups during the reaction.

Dendrimers

Dendrimers are macromolecular highly branched compounds formed by reiterative reaction sequences starting from an initial core molecule with successive layers or stages being added in successive “generations” to build up a three-dimensional, highly ordered polymeric compound. Dendrimers are characterized by the following features: i) an initiator core (I) which may have one or more reactive sites and be point-like or of significant size so as to effect the final topology of the dendrimer; ii) layers of branched repeating units (Z) attached to the initiator core; iii) functional terminal groups (such as moieties A) attached to the surface of the dendrimer, optionally through linking groups (such as linking groups X). In a non-limiting example, the present invention uses dendritic structures as frameworks for the attachment of peptide moieties. Furthermore, the invention is not limited to the spherical dendrimers but can be based on any dendritic structure. The variety of dendrimers in both shape and constitution are well known to persons skilled in the art. For example, PAMAM dendrimers represent an exciting new class of macromolecular architecture called “dense star” polymers. Unlike classical polymers, dendrimers have a high degree of molecular uniformity, narrow molecular weight distribution, specific size and shape characteristics, and a highly-functionalized terminal surface. The manufacturing process is a series of repetitive steps starting with a central initiator core. Each subsequent growth step represents a new “generation” of polymer with a larger molecular diameter, twice the number of reactive surface sites, and approximately double the molecular weight of the preceding generation.

The preparation of dendrimers is well known, and is described by way of example in U.S. Pat. Nos. 4,289,872 and 4,410,688 (describing dendrimers based on layers of lysine units), as well as U.S. Pat. Nos. 4,507,466, 4,558,120, 4,568,737 and 4,587,329 (describing dendrimers based on other units including polyamidoamine or PAMAM dendrimers). The dendrimers disclosed in these U.S. patents are described as being suitable for uses such as surface modifying agents, as metal chelating agents, as demulsifiers or oil/water emulsions, wet strength agents in the manufacture of paper, and as agents for modifying viscosity in aqueous formulations such as paints. It is also suggested in U.S. Pat. Nos. 4,289,872 and 4,410,688 that the dendrimers based on lysine units can be used as substrates for the preparation of pharmaceutical dosages.

International Patent Publications Nos. WO 88/01178, WO 88/01179 and WO 88/01180 disclose conjugates in which a dendrimer is conjugated or associated with another material such as a carried pharmaceutical or agricultural material. In addition, International Patent Publication No. WO 95/24221 discloses dendritic polymer conjugates composed of at least one dendrimer in association with a carrier material which can be a biological response modifier, and optionally a target director. These patent publications together with the U.S. patents mentioned above contain a broad disclosure of various dendrimers and processes for the preparation thereof, and the disclosure of each of these publications is incorporated herein by reference.

The term “dendrimer” as used herein is to be understood in its broadest sense, and to include within its scope all forms and compositions of these dendrimers as disclosed in Patent Publications Nos. WO 88/01178, WO 88/01179 and WO 88/01180. The term also includes linked or bridged dendrimers as disclosed in these patent publications.

In one particular embodiment, the dendrimers contemplated for use in the present invention are shown in Example 14. Other representative dendrimeric complexes are described in U.S. Pat. Nos. 6,949,620; 6,083,708; 7,005,124 and 7,005,123, all of which are incorporated by reference in their entireties. The preferred dendrimers of the present invention comprise a polyvalent core covalently bonded to at least two dendritic branches, and preferably extend through at least two generations. Particularly preferred dendrimers are polyamidoamine (PAMAM) dendrimers, PAMAM (EDA) dendrimers, poly(Propyleneimine) (PPI) dendrimers and polylysine dendrimers. In a more preferred embodiment, the dendrimer is polystyrene/maleic anhydride.

Candidate Compounds and Agents

Examples of agents, candidate compounds or test compounds include, but are not limited to, nucleic acids (e.g., DNA and RNA), carbohydrates, lipids, proteins, peptides, peptidomimetics, small molecules and other drugs. In one preferred aspect, agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Screening/Testing for Active Peptides and Peptide Mimics

Any screening technique known in the art can be used to screen for anti-HIV peptides or mimics of the gp340 polypeptide that inhibits HIV infectivity. The present invention contemplates screens for small molecule ligands or ligand analogs and mimics, as well as screens for natural ligands that bind to and antagonize such activity in vitro or in vivo. For example, natural products libraries can be screened using assays of the invention for molecules that have anti-HIV activity by virtue of their ability to bind to the gp120 HIV envelope protein, or peptides derived therefrom.

Identification and screening of molecule is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Another approach uses recombinant bacteriophage to produce large libraries. Using the “phage method” (Scott and Smith, 1990, Science 249:386-390 (1990); Cwirla, et al., Proc. Natl. Acad. Sci., 87:6378-6382 (1990); Devlin et al., Science, 249:404-406 (1990)), very large libraries can be constructed (10⁶-10⁸ chemical entities). A second approach uses primarily chemical methods, of which the Geysen method (Geysen et al., Molecular Immunology 23:709-715 (1986); Geysen et al. J. Immunologic Method 102:259-274 (1987)) and the method of Fodor et al. (Science 251:767-773 (1991)) are examples. Furka et al. (14th International Congress of Biochemistry, Volume 5, Abstract FR:013 (1988); Furka, Int. J. Peptide Protein Res. 37:487-493 (1991)), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In another aspect, synthetic libraries (Needels et al., Proc. Natl. Acad. Sci. USA 90:10700-4 (1993); Ohlmeyer et al., Proc. Natl. Acad. Sci. USA 90:10922-10926 (1993); Lam et al., International Patent Publication No. WO 92/00252; Kocis et al., International Patent Publication No. WO 9428028, each of which is incorporated herein by reference in its entirety), and the like can be used to screen for novel peptides or mimics thereof or fragments thereof according to the present invention.

Alternatively, assays for binding of natural ligand to cells that express recombinant forms of a gp340 protein binding domain can be performed. The soluble ligands can be provided readily as recombinant or synthetic proteins.

The screening can be performed with recombinant cells that express the gp340 polypeptide, or alternatively, using purified protein, e.g., produced recombinantly, as described above. For example, the ability of a labeled, soluble or solubilized gp340 peptide or mimic thereof or fragment thereof that includes the ligand-binding portion of the molecule, to bind ligand can be used to screen libraries.

Phage Display

Screening phage-displayed random peptide libraries offers a rich source of molecular diversity and represents a powerful means of identifying peptide ligands that bind a receptor molecule of interest (Cwirla, et al., Proc. Natl. Acad. Sci., 87:6378-6382 (1990); Devlin et al., Science, 249:404-406 (1990)). Phage expressing binding peptides are selected by affinity purification with the target of interest. This system allows a large number of phage to be screened at one time. Since each infectious phage encodes the random sequence expressed on its surface, a particular phage, when recovered from an affinity matrix, can be amplified by another round of infection. Thus, selector molecules immobilized on a solid support can be used to select peptides that bind to them. This procedure reveals a number of peptides that bind to the selector and that often display a common consensus amino acid sequence. Biological amplification of selected library members and sequencing allows the determination of the primary structure of the peptide(s).

Peptides are expressed on the tip of the filamentous phage M13, as a fusion protein with the phage surface protein pilus (at the N-terminus). Typically, a filamentous phage carries on its surface 3 to 5 copies of pili and therefore of the peptide. In such a system, no structural constraints are imposed on the N-terminus; the peptide is therefore free to adopt many different conformations, allowing for a large diversity. However, biases in the distribution of peptides in the library may be caused by biological selection against certain of the peptides, which could reduce the diversity of peptides contained in the library. In practice, this does not appear to be a significant problem. When randomly selected peptides expressed at the N-terminus of pili were analyzed (Cwirla, et al., Proc. Natl. Acad. Sci., 87:6378-6382 (1990)), most amino acids appeared at each position of the variable peptide, indicating that no severe discrimination against particular amino acids had occurred. Selection against particular combinations of amino acids would however not have been detected in this analysis.

Peptide ligands identified by phage display screening frequently interact with natural binding site(s) on the target molecule, and often resemble the target's natural ligand(s). Peptides that mimic HIV-associated carbohydrate forms have also been reported. Mouse antisera were generated against peptides that mimic a mucin-related carbohydrate epitope expressed on HIV. The authors showed that immunization with the peptide-mimics induces antibodies that cross-reacted with native HIV envelope proteins. The sera containing these antibodies could neutralise HIV-1 cell-free infection in vitro as well as the sera from patients infected with HIV-1 whereas normal human sera were ineffective in this viral neutralisation assay (Agadjanyan et al, 1997).

Pharmaceutical Compositions and Methods of Administration of Anti-Microbial Agents

The invention encompasses the preparation and use of pharmaceutical compositions comprising an agent, particularly a peptide or mimic thereof, useful for the prevention of HIV infection or inhibition of HIV infectivity as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

Such compositions comprise a therapeutically effective amount of the agents of the invention, particularly the peptides or mimics thereof, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

Pharmaceutical compositions containing the therapeutic agent, whether it be a polypeptide, analog or active fragment-containing compositions or small organic molecules, which are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The composition containing the therapeutic agent, may also be administered by various routes including intravenously, intramuscularly, subcutaneously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. Precise amounts of active ingredient required to be administered depend on the judgment of the practitioner and are peculiar to each individual. Suitable regimes for initial administration and subsequent injections are also variable, but are typified by an initial administration followed by repeated doses at intervals by a subsequent injection or other administration.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Administration of the compositions to the site of injury, the target cells, tissues, or organs, may be by way of oral administration as a pill, capsule or a tablet. A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion. As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Administration of the compositions to the site of injury, the target cells, tissues, or organs, may be by way of oral administration as a pill or capsule or a liquid formulation or suspension. It may be administered via the transmucosal, sublingual, nasal, rectal or transdermal route. Parenteral administration may also be via intravenous injection, or intramuscular, intradermal or subcutaneous. Due to the nature of the diseases or conditions for which the present invention is being considered, the route of administration may also involve delivery via suppositories.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

The compositions may be provided as a liposome formulation. Liposome delivery had been utilized as a pharmaceutical delivery system for other compounds for a variety of applications. See, for example Langer (1990) Science 249:1527-1533; Treat et al. (1989) in Liposomes in the Therapy of Infectious Disease and Cancer, Lopez-Berestein and Fidler (eds.), Liss: New York, pp. 353-365 (1989). Many suitable liposome formulations are known to the skilled artisan, and may be employed for the purposes of the present invention. For example, see: U.S. Pat. No. 5,190,762.

In a further aspect, liposomes can cross the blood-brain barrier, which would allow for intravenous or oral administration. Many strategies are available for crossing the blood-brain barrier, including but not limited to, increasing the hydrophobic nature of a molecule; introducing the molecule as a conjugate to a carrier, such as transferrin, targeted to a receptor in the blood-brain barrier; and the like.

Transdermal delivery of the plant compositions or extracts is also contemplated. Various and numerous methods are known in the art for transdermal administration of a drug, e.g., via a transdermal patch. It can be readily appreciated that a transdermal route of administration may be enhanced by use of a dermal penetration enhancer.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

Controlled release oral formulations may be desirable. The composition may be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Pulmonary delivery may be used for treatment as well. Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention.

Ophthalmic and nasal delivery may be used in the method of the invention. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextrins. For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

The compositions of the present invention are also suited for transmucosal delivery. In particular, the compositions and extracts are particularly suited for sublingual, buccal, vaginal or rectal delivery of agents that are sensitive to degradation by proteases present in gastric or other bodily fluids having enhanced enzymatic activity. Moreover, transmucosal delivery systems can be used for agents that have low oral bioavailability. The compositions of the instant invention comprise the plant extract dissolved or dispersed in a carrier that comprises a solvent, an optional hydrogel, and an agent that enhances transport across the mucosal membrane. The solvent may be a non-toxic alcohol known in the art as being useful in such formulations of the present invention and may include, but not be limited to ethanol, isopropanol, stearyl alcohol, propylene glycol, polyethylene glycol, and other solvents having similar dissolution characteristics. Other such solvents known in the art can be found in “The Handbook of Pharmaceutical Excipients”, published by The American Pharmaceutical Association and The Pharmaceutical Society of Great Britain (1986) and the Handbook of Water-Soluble Gums and Resins, ed. By R. L. Davidson, McGraw-Hill Book Co., New York, N.Y. (1980).

Any transmucosal preparation suitable for administering the components of the present invention or a pharmaceutically acceptable salt thereof can be used. Particularly, the mixture is any preparation usable in oral, nasal, vaginal or rectal cavities that can be formulated using conventional techniques well known in the art. Preferred preparations are those usable in oral, nasal or rectal cavities. For example, the preparation can be a buccal tablet, a sublingual tablet, and the like preparation that dissolve or disintegrate, delivering drug into the mouth of the patient. A spray or drops can be used to deliver the drug to the nasal cavity. A suppository can be used to deliver the mixture to the rectal mucosa. The preparation may or may not deliver the drug in a sustained release fashion. In one particular embodiment, the transmucosal formulation allows for delivery of the active ingredient to the site where needed, but does not promote entry of the active moiety into the bloodstream.

A specific embodiment for delivery of the components of the present invention is a mucoadhesive preparation. A mucoadhesive preparation is a preparation which upon contact with intact mucous membrane adheres to said mucous membrane for a sufficient time period to induce the desired therapeutic or nutritional effect. The preparation can be a semisolid composition as described for example, in WO 96/09829. It can be a tablet, a powder, a gel or film comprising a mucoadhesive matrix as described for example, in WO 96/30013. The mixture can be prepared as a syrup that adheres to the mucous membrane.

Suitable mucoadhesives include those well known in the art such as polyacrylic acids, preferably having the molecular weight between from about 450,000 to about 4,000,000, for example, Carbopol™934P; sodium carboxymethylcellulose (NaCMC), hydroxypropylmethylcellulose (HPMC), or for example, Methocel™ K100, and hydroxypropylcellulose.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, a gel or cream or solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject.

Douche preparations may further comprise various additional ingredients including, but not limited to, antioxidants, antibiotics, antifungal agents, and preservatives.

Additional delivery methods for administration of compounds include a drug delivery device, such as that described in U.S. Pat. No. 5,928,195.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intracranial, intraventricular, intrathecal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The delivery of the components of the present invention can also be accomplished using a bandage, patch, device, vaginal applicator, rectal applicator and any similar device that contains the components of the present invention and adheres to a mucosal surface. Suitable transmucosal patches are described for example in WO 93/23011, and in U.S. Pat. No. 5,122,127, both of which are hereby incorporated by reference. The patch is designed to deliver the mixture in proportion to the size of the drug/mucosa interface. Accordingly, delivery rates can be adjusted by altering the size of the contact area. The patch that may be best suited for delivery of the components of the present invention may comprise a backing, such backing acting as a barrier for loss of the components of the present invention from the patch. The backing can be any of the conventional materials used in such patches including, but not limited to, polyethylene, ethyl-vinyl acetate copolymer, polyurethane and the like. In a patch that is made of a matrix that is not itself a mucoadhesive, the matrix containing the components of the present invention can be coupled with a mucoadhesive component (such as a mucoadhesive described above) so that the patch may be retained on the mucosal surface. Such patches can be prepared by methods well known to those skilled in the art.

Preparations usable according to the invention can contain other ingredients, such as fillers, lubricants, disintegrants, solubilizing vehicles, flavors, dyes and the like. It may be desirable in some instances to incorporate a mucous membrane penetration enhancer into the preparation. Suitable penetration enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), and related compounds.

The administration of the compositions and extracts of the present invention can be alone, or in combination with other compounds effective at treating the various medical conditions contemplated by the present invention. Also, the compositions and formulations of the present invention, may be administered with a variety of analgesics, anesthetics, or anxiolytics to increase patient comfort during treatment.

The compositions of the invention described herein may be in the form of a liquid. The liquid may be delivered as a spray, a paste, a gel, or a liquid drop. The desired consistency is achieved by adding in one or more hydrogels, substances that absorb water to create materials with various viscosities. Hydrogels that are suitable for use are well known in the art. See, for example, Handbook of Pharmaceutical Excipients, published by The American Pharmaceutical Association and The Pharmaceutical Society of Great Britain (1986) and the Handbook of Water-Soluble Gums and Resins, ed. By R. L. Davidson, McGraw-Hill Book Co., New York, N.Y. (1980).

Suitable hydrogels for use in the compositions include, but are not limited to, hydroxypropyl cellulose, hydroxypropyl methyl cellulose, sodium carboxymethyl cellulose and polyacrylic acid. Preferred hydrogels are cellulose ethers such as hydroxyalkylcellulose. The concentration of the hydroxycellulose used in the composition is dependent upon the particular viscosity grade used and the viscosity desired in the final product. Numerous other hydrogels are known in the art and the skilled artisan could easily ascertain the most appropriate hydrogel suitable for use in the instant invention.

The mucosal transport enhancing agents useful with the present invention facilitate the transport of the agents in the claimed invention across the mucosal membrane and into the blood stream of the patient. The mucosal transport enhancing agents are also known in the art, as noted in U.S. Pat. No. 5,284,657, incorporated herein by reference. These agents may be selected from the group of essential or volatile oils, or from non-toxic, pharmaceutically acceptable inorganic and organic acids. The essential or volatile oils may include peppermint oil, spearmint oil, menthol, eucalyptus oil, cinnamon oil, ginger oil, fennel oil, dill oil, and the like. The suitable inorganic or organic acids useful for the instant invention include but are not limited to hydrochloric acid, phosphoric acid, aromatic and aliphatic monocarboxylic or dicarboxylic acids such as acetic acid, citric acid, lactic acid, oleic acid, linoleic acid, palmitic acid, benzoic acid, salicylic acid, and other acids having similar characteristics. The term “aromatic” acid means any acid having a 6-membered ring system characteristic of benzene, whereas the term “aliphatic” acid refers to any acid having a straight chain or branched chain saturated or unsaturated hydrocarbon backbone.

Other suitable transport enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), and related compounds.

When the compositions and extracts of the instant invention are to be administered to the oral mucosa, the preferred pH should be in the range of pH 3 to about pH 7, with any necessary adjustments made using pharmaceutically acceptable, non-toxic buffer systems generally known in the art.

For topical delivery, a solution of the peptide or mimic thereof in water, buffered aqueous solution or other pharmaceutically-acceptable carrier, or in a hydrogel lotion or cream, comprising an emulsion of an aqueous and hydrophobic phase. To this may be added ascorbic acid or its salts, or other ingredients, or a combination of these, to make a cosmetically-acceptable formulation. Metals should be kept to a minimum. It may be preferably formulated by encapsulation into a liposome for oral, parenteral, or, preferably, topical administration. The topical composition of the invention preferably comprises the composition in an amount of between about 0.1% and about 15%, more preferably 1% to 15%, more preferably 2.5% to 15% and most preferably about 5% to 10%.

As used herein the term “topical composition” refers to a composition which is suitable for application to the surface of a body part, or a localized area of the body. Preferably the surface of a body part comprises skin or a mucous membrane. In a preferred topical composition, the active moiety may be prepared as a cream, lotion or gel that contains any one or more of the following components in the formulation: glycerin, gelatin, hydroxyethyl cellulose, methyl or ethyl cellulose, starch, carbopol, or carboxy methyl cellulose (CMC).

The invention provides methods of treatment comprising administering to a subject a therapeutically effective amount of at least one of the peptides or a mimic thereof. In one embodiment, the peptide is substantially purified (e.g., substantially free from substances that limit its effect or produce undesired side-effects). The subject is preferably an animal, including but not limited to primates, including monkeys, and most preferably human. In one specific embodiment, a non-human mammal is the subject. In another specific embodiment, a human mammal is the subject.

The amount of the peptide which is optimal in treating a retrovirus infection or treating immune deficiencies can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

The compound may be administered to an animal, more particularly, a human, as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The compound may also be administered as slow release by way of a vaginal ring (See U.S. Pat. No. 5,928,195).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Effective Doses

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Normal dose ranges used for particular therapeutic agents employed for specific diseases can be found in the Physicians' Desk Reference, 54^{th} Edition (2000).

### EXAMPLES

The following examples are presented in order to more fully illustrate the preferred embodiments of the invention. They should in no way be construed, however, as limiting the broad scope of the invention.

### Example 1A

### Polymeric Constructs using Reactive Maleic Anhydride Polymers and Tetrameric Peptides

Materials and Methods

Preparation of Polymeric Complexes of Polymaleic Anhydride (PMA) Polymers and Tetrameric Peptides

The monomers used in this study were tetramer peptides WRWR-NH₂ and WWRR—NH₂, respectively. Tryptophan- and arginine-rich peptides, whether linear or branched, are known to display membrane disruption or fusion properties that have been exploited for antibacterial activity. (Strom M. B.; Rekdal, O.; Svendsen, I. S. Antimicrobial activity of short arginine- and tryptophan-rich peptides. J. Pept. Sci. 2002, 8 (8), 431-437; Wessolowski, A.; Bienert, M.; Dathe, M. Antimicrobial activity of arginine- and tryptophan-rich hexapeptides: the effects of aromatic clusters, D-amino acid substitution and cyclization. J. Pept. Res. 2004, 64 (4), 159-169). Recently, R- and W-containing antimicrobial pentapeptides and even dipeptides derived from antibiotic core peptide of bovine lactoferricin are noted as good short cationic antibacterial peptides. (Strom, M. B.; Haug, B. E.; Skar, M. L.; Stensen, W.; Stiberg, T.; Svendsen, J. S. The pharmacophore of short cationic antibacterial peptides. J. Med. Chem. 2003, 46 (9), 1567-1570).

While the above is presented with the WRWR tetramer, it can be visualized that the same sequence of reactions proceeds with the WWRR tetramer, or any peptide encompassed within the scope of the present invention, as well.

As noted above, two tetramer peptides were used that have moderate antimicrobial activity-WRWR—NH₂ and RWRW—NH₂. Peptide monomers were synthesized using standard FMOC solid-phase chemistry, and the N termini were capped for comparison with the polymeric species. The peptides were linked to a 5000 MW polymaleic anhydride matrix to produce more highly multivalent complexes. More particularly, PMA is considered to have an average molecular weight of 5000, which constitutes an average of 50 maleic anhydride units per polymer chain (according to the manufacturer), and PEMA has average 400 maleic anhydride units per polymer chain with molecular weight of 100,000. The conjugation reaction between the polymer and the peptides was agitated overnight at 30° C. on a rotary shaker in DMF or DMSO with proper catalyst, such as diisopropylethylamine (DIEA). The remaining maleic anhydride groups were quenched with the addition of distilled water, and the product was treated with excess of trimethylsilyldiazomethane (TMDM) to methylate any carboxylic acid groups. Solutions were dialyzed overnight (5000 mwco, 1000 mL) in distilled water, and compound was isolated. Control polymer was generated by the addition of methylamine in place of peptides. The peptide RRWW was prepared as the complex PMX-1, and the peptide RWRW was prepared as PMX-2. The general structure of the complex is shown below:

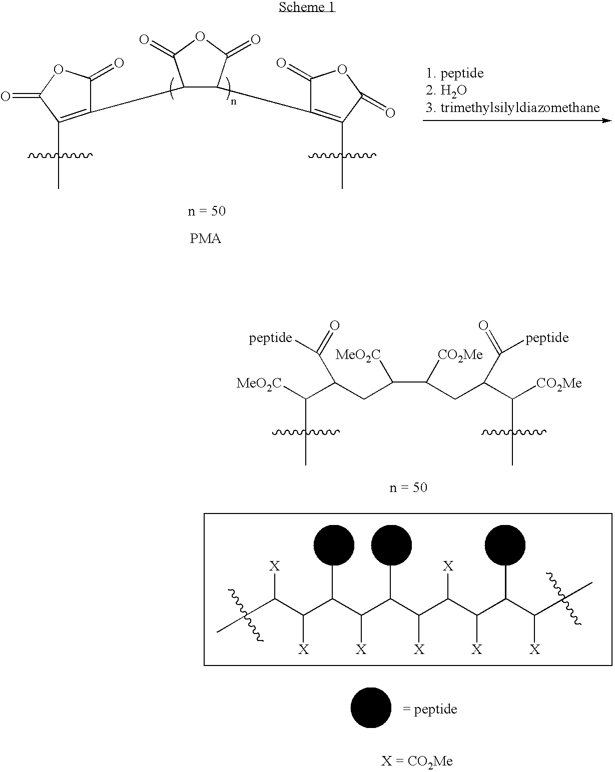

In the following scheme, the preparation of a complex with an ethylene/maleic anhydride copolymer (PEMA) is illustrated. 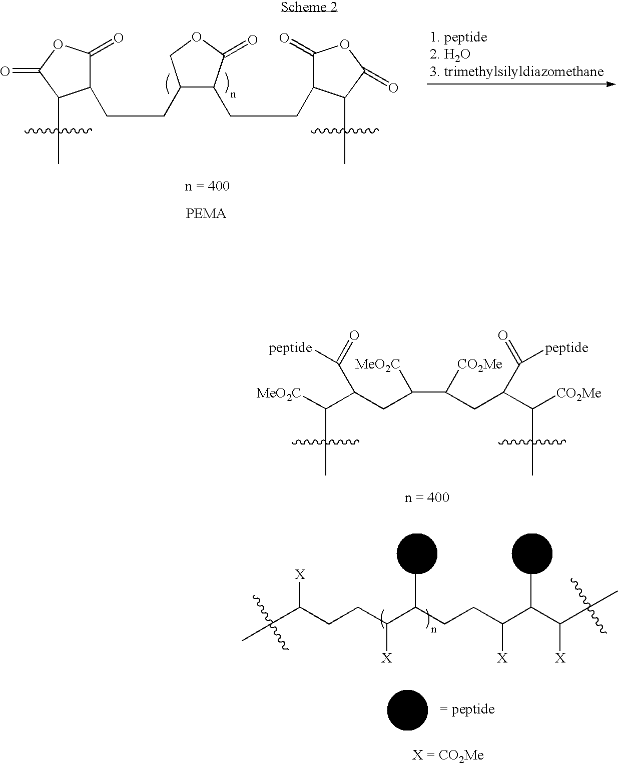
Antibacterial Testing

Antibacterial activity was carried on both Gram-negative bacteria E. coli, and Gram-positive bacteria B. subtilis. Minimum inhibitory concentration (MIC) was defined as ten percent growth inhibition, compared to positive growth controls. Cultures were grown overnight on Mueller Hinton (MHB) agar plates, and 3 to 5 uniform colonies of the given organism were transferred with an inoculating loop to test tubes containing approximately 6 mL of Tryptic Soy Broth (TSB). The tubes were then incubated at 37° C. for 1½ to 2 hours, until the turbidity of the cell suspension matched that of the 0.5 McFarland standard. At this point, 50 μL of the standardized culture was added to each well of a 24-well plate, along with 800 μL of MHB. Different volumes of a stock concentration of peptide were then added, followed by 0.1 M phosphate buffered saline (PBS) to bring the total final volume to 1 mL, giving a variety of test concentrations of peptide. For a given bacterial strain, each concentration was repeated for four different wells. Control conditions consisted of 50 μL of standardized culture in TSB, 800 μL of MHB, and 150 μL of PBS.

The plates were incubated with shaking for 6 hrs to insure the cultures had reached log phase growth. After the given incubation time, the plates were removed, and the liquid from each well transferred to a 1.5 mL cuvette for absorbance readings at a wavelength of 600 nm. A Genesys 5 Spectrophotometer (Thermo Spectronic) was used, and a 1 mL sample containing 800 μL of MHB, 150 μL of PBS and 50 μL TSB served as a blank to calibrate the machine. The 6-hour absorbance data were used to calculate the percent inhibition for each test condition by comparing to the absorbance of the control.

Hemolysis Assays

Hemolytic activity of the peptides was determined on a 10% suspension of sheep red blood cells in 10 mM Tris-HCl buffer, 150 mM NaCl, pH 7.0. 800 μL of this suspension were mixed in 1.5 mL Eppendorf tubes with varying volumes of peptide stock and buffers for a total volume of 1 mL. These tubes were then incubated at 37° C. for 1 hr. After the hour, the tubes were spun down at 3,000 rpm on a table top centrifuge for 5 mins, and the resulting supernatant was diluted by a factor of 40 in distilled water. The absorbance of the dilutions at 540 nm was measured in the Genesys 5 Spectrophotometer (Thermo Spectronic). Zero hemolysis and 100% hemolysis controls were determined by incubating the cells with buffer and 20% Tween, respectively. The final concentrations of peptides were designed to approximate 0.1×, 1×, 2×, 5×, and 10× of the MIC value for growth inhibition, to evaluate the potential therapeutic ratio.

Antibacterial activity was tested towards Gram-negative Escherichia coli and Gram-positive Bacillus subtilis. Minimum inhibitory concentration (MIC) was defined as the concentration of peptide which resulted in 10% inhibition of growth. Hemolytic activity of the peptides was tested toward a sheep red blood cell suspension, as described earlier. The results of these tests are set forth in Table 2, below.

TABLE 2

| | MIC | MIC | Hemolytic Activity | |
|---|---|---|---|---|
| | E. coli | B. subtilis | (10%) | |
| Antimicrobial agents | μg/mL | μg/mL | μg/mL | |
| Monomeric RRWW—NH₂ | 453 | 100 | 1250 | |
| Monomeric RWRW—NH₂ | 260 | 90 | 2250 | |
| RRWW—NH₂ on PMA | 55 | 12 | 250 | |
| RWRW—NH₂ on PMA | 25 | 9 | 230 | |
| Control polymer | N/A | N/A | >1000 | μg/mL |

*N/A means control polymer has no activity on bacteria

Results

The polymers therefore enhance the activity of monomers-per gram of compound the increase is notable while the hemolytic index remains constant. This means we have improved the effective activity by a factor of about 10 calculated on a mass basis. If we calculate the effect on a molar basis, we still see a factor of 10 improvement.

Conclusions

In summary, it has been demonstrated that the polymers of the invention, such as PMA and PEMA, can be used as a reactive substrate to construct the present polyvalent polymer complexes, and that the complexes are significantly more effective than monomer elements alone. This polyvalent antibacterial polymer design takes advantage of the amplification by polyvalency, and increases the effective molarity of the monomeric units while decreasing the entropy of self assembly, so that the hydrophobic clusters on a peptide polymer lead to aggregation that may enhance fusogenic activity (Tam, J. P.; Lu, Y. A,; Yang, J. L. Antimicrobial dendrimeic peptides. Eur. J. Biochem. 2002, 269 (3), 923-932). In the most commonly suggested mechanisms for membrane disruption based on amphiphilic peptides, there is some type of cooperative action, in either pore formation or coverage of the surface in a carpetlike manner (Zasloff, M. Antimicrobial peptides of multicellular organisms. Nature 2002, 415 (6870), 389-395; Shai, Y. Mechanism of the binding, insertion and destabilization of phospholipid bilayer membranes by alpha-helical antimicrobial and cell non-selective membrane-lytic peptides. Biochitn. Biophys. Acta 1999, 1462 (1-2), 55-70).

The present polymer product is speculated to mimic the mechanisms of action through which high-ordered antimicrobial peptides exert their membranolytic effects. Thus, the short antibacterial peptides used and demonstrated herein, may represent a useful building block for unusual biopolymer design that offers various advantageous membranolytic activities in lipid environments.

Moreover, our experience shows that production of peptides on a commercial scale is feasible. The monomer is small and cheap, as the peptides average less than 5 residues. With the present procedure, the polyvalent antibacterial polymer is easy to prepare in large quantities.

Discussion

The polyvalent polymer strategy described here may be generally applicable for creating scaffolds to enhance surface interactions of a variety of peptides, including cationic antimicrobial peptides. In such a scaffold, the spacing, relative positioning and relative orientation of monomer elements may be important.

From the foregoing description, various modifications and changes in the compositions and methods of this invention will occur to those skilled in the art. All such modifications coming within the scope of the appended claims are intended to be included therein.

It is further understood that all base sizes or amino acid sizes, and all molecular weight or molecular mass values, given for nucleic acids or polypeptides are approximate, and are provided for description.

All publications, including but not limited to patents and patent applications, cited in this specification are herein incorporated by reference as if each individual publication were specifically and individually indicated to be incorporated by reference herein as though fully set forth.

### Example 1B

### Multivalent Antimicrobial Peptides from a Reactive Polymer Scaffold Preparation of Monomeric Peptides

In this example, the procedures and schemes employed in Example 1A were used. Thus, the preparation of the peptide-polymeric complexes are shown in Schemes 1A and 2A. Monomeric peptide RWRW—NH₂ and RRWW—NH₂ were assembled on Rink amide resin (Nova Biochem, Corp., CA) using a RAININ Instrument PS3 solid phase synthesizer and Fmoc (9-fluorenylmethyloxycarbonyl) chemistry. Fmoc-Trp(tBoc)/Arg(Pbf), the coupling reagent HBTU (2-(1H-benzotriazol-1-yl) 1,1,3,3-tetramethyluroniumhexafluoro phosphate) and HOBT (N-hydroxybenzotriazol) were also purchased from Nova Biochem. The N terminus of each peptide was capped with acetic anhydride after its assembly on the solid matrix. Cleavage of peptides from the resin was achieved with 95% trifluoroacetic acid (TFA) in the presence of the scavenger2.5% TIS (triisopropylsilane) and 2.5% H₂O. After precipitation with cold ether, crude samples were purified on a reverse-phase HPLC C-18 preparative column (2.2×25 cm, 300 Å, Grace Vydac, Hesperia, Calif.) with water and acetonitrile as eluents, using a 0-60% B linear gradient over 30 min. Fractions containing >99% of the expected peptide, according to analytical HPLC were lyophilized. High-resolution mass spectra (HRMS) were obtained on a LC/MSD TOF (Agilent Technologies). LRMS data was obtained on an Agilent 1100 series LC/MSD (XCT) electrospray trap. LRMS m/z for Ac—RWRW—NH₂, [M]⁺ calcd 744.4, found 744.5; Ac—RRWW—NH₂, [M]⁺ calcd 744.4, found 744.5.

Preparation of Monomeric Peptides with Side Chain Protecting Groups

Peptide R(Pbf)W(tBoc)R(Pbf)W(tBoc)-NH₂ and R(Pbf)R(Pbf)W(tBoc)W(tBoc)-NH₂ were assembled on Sieber Amide resin (Nova Biochem, Corp., CA) by using the same synthesis as above but without capping N terminus at the last step of synthesis. After completion of synthesis, peptides were cleaved from resin with 1% TFA in dichloromethylene. This gave the peptide product with side chain-protecting groups on to avoid cross-linking in further reactions (Chan, W. C.; Mellor, S. L. Reductive Alkylation Of 9-Amino-Xanthen-3-Yloxymethylpoly(Styrene)—A Novel Procedure For The Synthesis Of Peptidyl N-Alkyl Amides By Fmoc/Bu(T) Chemistry. J. Chem. Soc., Chem. Commun., 1995(14): p. 1475-77). Crude product containing >95% of the expected peptide, according to analytical HPLC, was used for preparation of multivalent peptides without further purification. LRMS m/z for R(Pbf)W(tBoc)R(Pbf)W(tBoc)-NH₂ [M]⁺ calcd 1406.66, found 1407.3; R(Pbf)R(Pbf)W(tBoc)W(tBoc)-NH₂, [M]⁺ calcd 1406.66, found 1407.3.

Preparation of Multivalent Peptides

The PMA sample had an average molecular weight of 5000 (according to the manufacturer, Polysciences, Inc., PA), confirmed by GPC, corresponding to an average of 50 maleic anhydride units per polymer chain. The starting PMA polymer is moderately polydisperse, with a polydispersity index of 2.1, measured by light scattering (Dynapro, Protein Solutions, Ltd., UK). Free COOH wasn't detected by ¹H NMR spectra.

The conjugation reaction between PMA (15 mg, 50 mg/ml) and 100 equivalent excess peptides (50 mg, 100 mg/ml) was carried out overnight at 40° C. in DMF with agitation on a rotary shaker catalyzed by diisopropylethylamine (DIEA) (100 equiv). The unreacted maleic anhydride groups were quenched by the addition of 150 μl distilled and deionized water, and then the product was treated with excess trimethylsilyldiazomethane (TMDM) to methylate the resulting carboxylic acid groups from either unreacted or reacted anhydride according to literature (Gestwicki, J. E.; Cairo, C. W.; Strong, L. E.; Oetjen, K. A.; Kiessling, L. L. Influencing receptor-ligand binding mechanisms with multivalent ligand architecture. J Am Chem Soc, 2002. 124(50): p. 14922-33). Free COOH wasn't detected by ¹H NMR spectra. After final solutions were dialyzed overnight (5000 mwco, 1000 mL) in distilled water, polymeric peptides were isolated. Control polymers were generated by the addition of 100 equivalent excess methylamine solution in THF (Sigma-Aldrich Co., MO) instead of peptides and isolated by steps described above. The cleavage of side chain-protecting groups from the polymer was performed with 95% TFA, 2.5% TIS and 2.5% H₂O. After precipitation with cold ether and overnight lyophilization, final polymer products were obtained (stepwise reaction can be seen in scheme 1A). The composition of the product was confirmed by ¹H NMR (Bruker 400 MHz, acetonitrile-d₃). PMA-RWRW, δ1.40 (m, 2H, R_{γ}H), δ1.80 (m, 2H, R_{β}H), δ2.0˜2.4 (m, nH, PMA (CH—CH)ₙ), δ2.90 (m, 2H, R_{δ}H), δ3.10 (m, 2H, W_{β}H), δ3.50˜3.70 (S, 3H, CO₂Me), δ4.05 (m, 1H, R_{α}H), δ4.30 (m, 1H, W_{α}H), δ6.6˜7.4 (R N_{ε}H, H in W ring, PMA end vinylene-keto group, terminal amide of C termini), δ7.70 (d, 1H. R NH), δ8.10 (d, 1H, W NH), δ9.95 (d, 1H, W NH). PMA-RRWW, δ1.40 (m, 2H, R_{γ}H), δ1.80 (m, 2H, R_{β}H), δ2.0˜2.4 (m, nH, PMA (CH—CH)ₙ), δ2.90 (m, 2H, R_{δ}H), δ3.10 (m, 2H, W_{β}H), δ3.50˜3.70 (S, 3H, CO₂Me), δ4.05 (m, 1H, R_{α}H), δ4.30 (m, 1H, W_{α}H), δ6.6˜7.4 (R N_{ε}H, H in W ring, PMA end vinylene-keto group, terminal amide of C termini), δ7.70 (d, 1H, R NH), δ8.10 (d, 1H, W NH), δ9.95 (d, 1H, W N_{ε}H). PMA control, δ2.0˜2.4 (m, nH, PMA (CH—CH)ₙ), δ2.6-2.9 (m, 3H, N—CH₃), δ3.50˜3.75 (S, 3H, CO₂Me), δ7.2 (S, 2H, PMA end vinylene-keto group), δ8.0 (m, 1H, NH).

Characterization of Multivalent Peptides

The extent of substitution on each polymer was estimated by integration of ¹H NMR peaks from multivalent peptide. NMR samples were prepared by dissolving about 5 mg of polypeptide into 0.6 mL acetonitrile-d₃. The NMR resonances used for composition determination were peak A at δ1.80 ppm (assigned to be PCH₂ of Arg residues in peptides) and peak B at δ3.50˜3.70 (assigned to be CO₂Me). The integrals of these two peaks were used to determine the substitutive ratio, giving the results of calculated molecular weight and monomer content in PMA in Table 3. For control polymers, proton chemical shifts δ2.6-2.9 (assigned to be N-methyl) and δ3.50˜3.75 (assigned to be CO₂Me) were used to determine the substitutive ratio. The result showed an average of 50 sites per PMA chain coupled to methylamine, which gave the calculated molecular weight of 7250 Da.

The molecular weight distribution of each of the polymers was determined by gel permeation chromatography system including a Beckman Coulter 127S solvent module pump, model 166 absorption detector and a calibrated TSK-GEL column (7.8×300 mm) packed with SDVB (Styrene Divinyl Benzene) for size-based separations (Tosoh Bioscience, LLC., CA). Polystyrenes samples (Polymer Laboratories, Inc., MA) of different molecular weight were used as standards and THF at a 1.0 mL/min flow rate was used as a mobile phase throughout the analysis. The peak average molecular weight (Mp) of the polymeric peptides relative to standards is listed in Table 3 without further calibration. Control polymers were measured with a molecular weight of 6750 Da. PMA-RWRW was characterized by ¹H NMR (Bruker 400 MHz, acetonitrile-d₃). The chemical shifts used for composition determination were peak A at δ1.80 ppm (assigned to be βCH₂ of Arg residue in peptides) and peak B at δ3.50˜3.70 (assigned to be CO₂Me)

Antibacterial Assays

The antimicrobial activity of each peptide was tested by following standard broth microdilution protocols recommended by the National Committee for Clinical Laboratory Standard (National Committee for Clinical Laboratory Standards. 2004, Methods for dilution antimicrobial susceptibility tests for bacteria that grow aerobically; sixth edition, approved standard M100-S14. National Committee for Clinical Laboratory Standards. Wayne, Pa.). Ampicillin- and streptomycin-resistant strain E. coli (D31) were obtained from the E. coli Genetic Resource Center (Yale University, New Haven, Conn.). E. coli (ATCC 25922), B. subtilis (ATCC 6633) and multi-drug resistant strain S. aureus (ATCC BAA-44) were obtained from American Type Culture Collection (ATCC; Rockville, Md.). B. anthracis (Sterne 34F2) is a gift from Dr. Martin J. Blaser in New York University School of Medicine. Monomeric peptides and multivalent ones were dissolved in DMSO/water to make series of stocks of 2-fold dilution. Cultures were grown overnight on Mueller Hinton (MH) agar plates, and 3 to 5 uniform colonies of each organism were transferred with an inoculating loop to test tubes containing MH broth, and incubated at 37° C. for 1.5 to 2 hours, until the turbidity matched that of the 0.5 McFarland standard (approximately 1.5×10⁸ cells/mL). 800 μL of MH broth and 50 μL of the bacterial inoculums were incubated in 24-well plates with varying volumes of stock solution and PBS buffer to bring the total volume to 1 mL. Solutions containing the same volumes of DMSO without compounds were used as controls. After these plates were incubated with shaking for 18 hrs at 35° C. to ensure the cultures had reached log phase growth, the liquid from each well were transferred to a 1.5 mL cuvette for absorbance readings at a wavelength of 600 nm using a Genesys 5 Spectrophotometer (Thermo Spectronic, Inc., NY). The 18-hour absorbance data were used to calculate the percent inhibition for each test condition by comparing with the absorbance of 1 mL samples with 800 μL of MH broth and 200 μL PBS. All assays were carried out in triplicate. The concentration of peptide that resulted in 50% inhibition of growth was recorded as IC50.

Hemolysis Assays

Stock solutions of samples were prepared by the same procedure as in antibacterial assays. Hemolytic activity of monomeric peptides and multivalent ones were assessed on fresh sheep erythrocytes (Fitzgerald, Inc., MA) in 35 mM PBS buffer, 150 mM NaCl, pH 7.0. Peptide concentrations corresponding to 50% hemolysis were used as hemolytic dose (HD50) determined from dose-response curve (National Committee for Clinical Laboratory Standards. 2004, Methods for dilution antimicrobial susceptibility tests for bacteria that grow aerobically; sixth edition, approved standard M100-S14. National Committee for Clinical Laboratory Standards. Wayne, Pa.). Aliquots of cell suspension were mixed in eppendorf tubes with varying volumes of stocks and buffer for a total volume of 1 mL. Solutions containing same volume of DMSO without samples were used as control. These tubes were incubated at 37° C. for 1 hr, and then spun down at 3,000 rpm for 5 mins. The resulting supernatant was diluted by a factor of 40 in distilled water. The absorbances of the dilutions at 540 nm were measured in the Genesys 5 Spectrophotometer (Thermo Spectronic, Inc., NY). Zero hemolysis and 100% hemolysis controls were obtained by incubating the cells with buffer and 20% Tween, respectively. Hemolytic index (HI) was defined as HI=HD50/IC50.

Preparation of the Polymer Peptide Complex

The monomers used in this study were tetrameric peptides RWRW—NH2 and RRWW—NH2, which have moderate activity and were designed with a balanced content of charged and bulky/lipophilic groups with an amidated C termini. The polymer used in this study was PMA (see scheme 1A); a copolymer of polyethylene and maleic anhydride (PEMA) has been used successfully (see scheme 2A) to display sugars for recognition by a cell surface protein, the lectin concanavalin A. As noted previously, peptide monomers were synthesized using standard FMOC solid-phase chemistry, and the N termini were capped for comparison with the polymeric species. PMA was supplied with an average molecular weight of 5000, or 50 maleic anhydride units per chain (according to manufacturer specifications). The multivalent AMPs were derived by conjugation of the maleic anhydride moieties with the free N termini of monomeric peptides in which all side chains were protected to avoid crosslinking. After the reaction was quenched with H20, the resulting carboxylate groups from unreacted or reacted anhydride were methylated by excess trimethylsilyldiazomethane (the stepwise reaction is summarized in Scheme 1A). The final polymer product has a series of branches that are peptide or carboxymethyl groups (free carboxylate groups were not detected), confirmed by ¹H NMR analysis. As a control for the polymer backbone itself, a sample of the polymer was fully substituted by addition of methylamine instead of peptide. The monomer ratio in each polymer was calculated by integration of ¹H NMR resonances: peak A at 1.80 ppm (assigned to P—CH2 of Arg side chains in peptides) and peak B at 3.50-3.70 ppm (assigned to C02Me). The ratio of these two peaks was used to determine the extent of peptide substitution, giving the calculated molecular weight and monomer content shown in Table 3. The methods for determining the effect on bacterial growth are described above for Example 1A. In addition, the IC₅₀ was also determined (the concentration of peptide complex sufficient to inhibit the growth of bacteria by 50%). 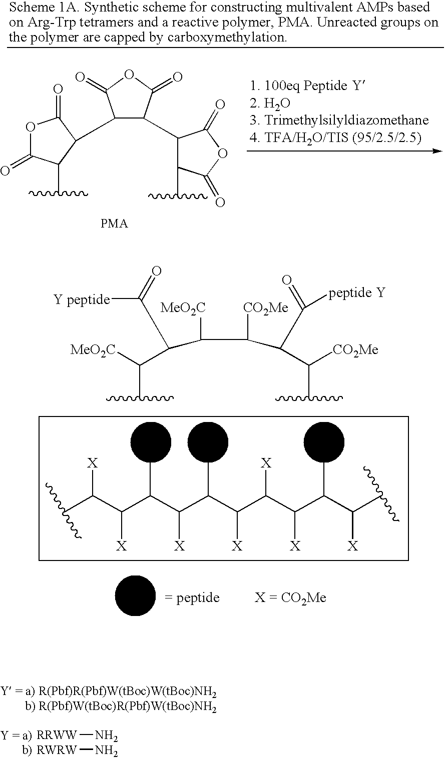 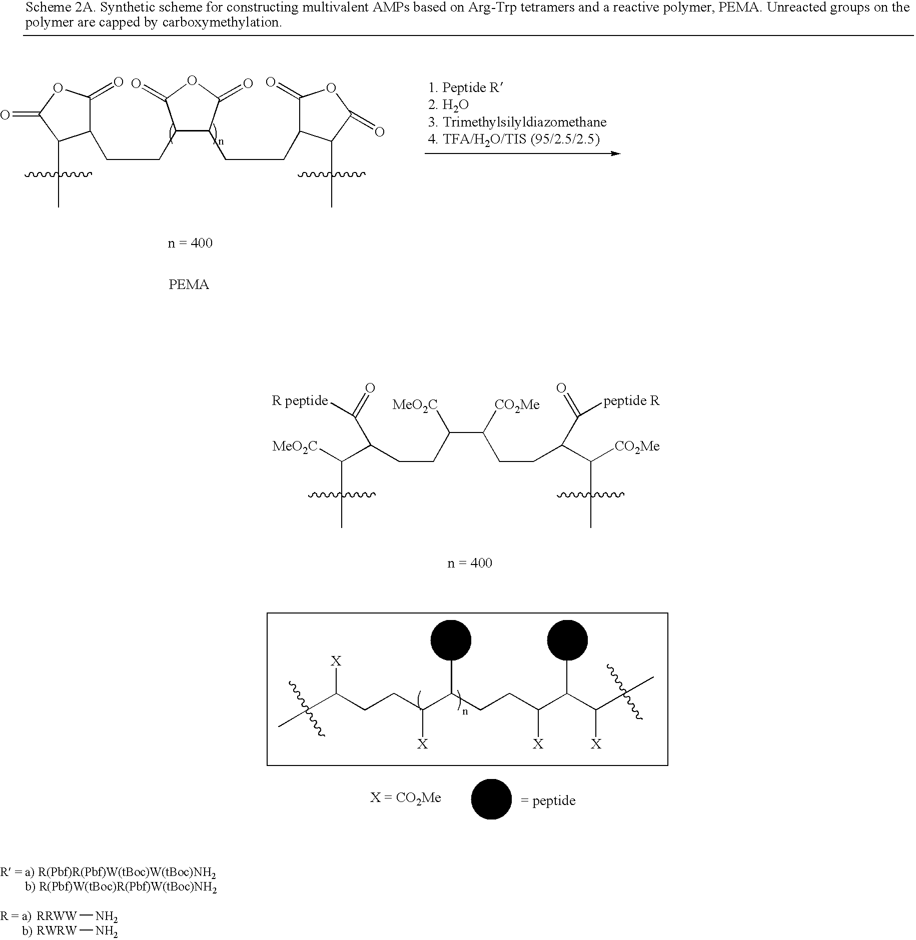
Results

The results showed that an average of 40 sites per PMA chain were coupled to peptides. The molecular weight distribution in each of the polypeptides was determined using gel permeation chromatography. The peak average molecular weight (Mp) of the polymeric peptides relative to standards is also listed in Table 3. Table 4 compares data on monomeric and polymeric peptides. The MIC values of the tetrapeptides in these experiments confirm that RWRW and RRWW are active antimicrobials despite their size. We found significant enhancement of antibacterial effect relative to the free peptides in polymers against the Gram negative bacteria E. coli and the Gram-positive bacteria B. subtilis, roughly a 10-fold improvement. There is a concomitant increase in hemolytic activity on fresh red blood cells, compensated by the reduction in IC₅₀ so that the ratio, expressed as the hemolytic index HI, remains roughly constant. The PMA control showed no activity against bacteria and low hemolytic activity. RWRW appears to be slightly more effective in polymers than RRWW. A QSAR analysis of the antimicrobial activity of R and W peptides suggests that charge and multiple W side chains are major characteristics while sequence is less important. It has also been previously shown that connecting several weakly interacting ligands on cell surfaces can lead to large increases in avidity (Mammen, M., Choi, S. K. and Whitesides, G. M. (1998). Polyvalent interactions in biological systems: Implications for design and use of multivalent ligands and inhibitors. Angewandte Chemie-International Edition 37(20): 2755-2794; Kiessling, L. L., Gestwicki, J. E. and Strong, L. E. (2000). Synthetic multivalent ligands in the exploration of cell-surface interactions. Curr Opin Chem Biol 4(6): 696-703). Dendrimeric anti-microbial peptides (AMPS) constructs that contain a maximum of eight monomer units showed improved solubility, salt resistance, and stability to proteolysis (Tam, J. P.; Lu, Y. A,; Yang, J. L. Antimicrobial dendrimeic peptides. Eur. J. Biochem. 2002, 269 (3), 923-932). A concern with multivalent strategies with respect to AMPS is to avoid exacerbating cytotoxic effects that can easily outweigh the positive effects on killing bacteria (Sal-Man, N.; Oren, Z.; Shai, Y. Preassembly of membrane-active peptides is an important factor in their selectivity toward target cells. Biochemistry 2002, 41 (39), 11921-11930). We show here that it is possible to decrease the IC₅₀ value of a moderately active tetrapeptide by roughly 1 order of magnitude using multivalent constructs while preserving HI values. The antibacterial polymeric peptides of this study increase in overall cationic charge as the number of monomeric peptides increases, while variations in their hydrophobicity depend on the conformation. Current mechanisms postulate that membrane disruption by AMPs involves cooperativity, in the process of assembling intermediates or overcoming repulsion of neighboring bound peptides (Zasloff, M. Antimicrobial peptides of multicellular organisms. Nature 2002, 415 (6870), 389-395; Lee, M. T.; Chen, F. Y.; Huang, H. W. Energetics of pore formation induced by membrane active peptides. Biochemistry 2004, 43 (12), 3590-3599; Shai, Y. Mechanism of the binding, insertion and destabilization of phospholipid bilayer membranes by alpha-helical antimicrobial and cell non-selective membrane-lytic peptides. Biochitn. Biophys. Acta 1999, 1462 (1-2), 55-70). Dendrimeric or polymeric mimics offer the potential to increase the local concentration of monomeric units while decreasing the entropy of self-assembly (Tam, J. P.; Lu, Y. A,; Yang, J. L. Antimicrobial dendrimeic peptides. Eur. J. Biochern. 2002, 269 (3), 923-932). On the other hand, if peptide assembly is required to prenucleate local structures that induce membrane lysis, then killing by free peptides should be stimulated by subcritical concentrations of polymer. To test this prediction, we added varying amounts of monomeric RWRW to 0.3× of the MIC of the polymeric peptide. Interestingly, we detected no influence of the polymer on killing by the monomeric peptides in these assays (data not shown). Thus, monomers do not appear to interact with the polymers and the two may act via different pathways. In any case, membrane target models for action by AMPs are likely to be oversimplified; interaction with negatively charged polymers of the bacterial cell wall has been implicated in some AMPs. (Matsuzaki, K.; Sugishita, K.; Miyajima, K. Interactions of an antimicrobial peptide, magainin 2, with lipopolysaccharide-containing liposomes as a model for outer membranes of Gram-negative bacteria. FEBS Lett. 1999, 449 (2-3), 221-224; Li, P.; Wohland, T.; Ho, B.; Ding, J. L. Perturbation of lipopolysaccharide (LPS) micelles by Sushi 3 (S3) antimicrobial peptide. The importance of an intermolecular disulfide bond in S3 dimer for binding, disruption, and neutralization of LPS. J. Biol. Chem. 2004, 279 (48), 50150-50156). The mechanism of multivalent AMPs and their activities in high salt and proteolysis conditions are currently under study.

Summary

In this work, we show that PMA affords a practical reactive scaffold for the assembly of multivalent AMPs that are more effective on a weight basis than the individual monomers. The monomers are small and relatively inexpensive to synthesize and could be derived from de novo design or fragments of natural AMPs in future work. In both sequences we tested (RWRW and RRWW), we detect enhanced antimicrobial activity, with an increase of roughly 10-fold in potency against Gram-negative and Gram-positive strains. At the same time the hemolytic activity of the polymers increases such that the hemolytic index remains roughly constant. Thus, a multivalent strategy based on PMA or related reactive polymers may offer a strategy for producing effective antibacterial agents in large quantities. Even if these are restricted to topical applications, they may prove to have practical utility as microbicides.

TABLE 3
Molecular Weight and Monomer Content in PMA

molecular weight (Da)

| compd | calcd | obsd^{a} | monomer content |
|---|---|---|---|
| PMA-RRWW | 33 940 | 31 000 | 40 |
| PMA-RWRW | 33 254 | 30 500 | 39 |

^{a}The peak average molecular weight (Mₚ).

TABLE 4
Summary of Bioassay Results with Multivalent AMPs and
Monomeric Peptides

| | IC₅₀,^{a} | | | hemolytic index | |
|---|---|---|---|---|---|
| antimicrobial | μg/mL | | HD₅₀, μg/mL | HI | |
| agents | E. coli | B. subtilis | red blood cell | E. coli | B. subtilis |
| AcRWRW- | 510 | 142 | 1850 | 3.6 | 13.0 |
| NH₂ | | | | | |
| AcRRWW- | 586 | 131 | 2280 | 3.9 | 17.4 |
| NH₂ | | | | | |
| PMA- | 33 | 10 | 128 | 3.9 | 12.8 |
| RWRW | | | | | |
| PMA- | 39 | 12 | 135 | 3.5 | 11.2 |
| RRWW | | | | | |
| PMA control | NA^{b} | NA^{b} | >1000 | NA | NA |

^{a}The results are the mean of three independent experiments each performed in parallel.
^{b}No killing was detected.

### Example 2

### Multivalent Design of Antimicrobial Peptides with Dendrimeric Structure

Synthesis and Characterization of Dendrimeric Antimicrobial Peptide

(Ac—WRWR)₄-lys₂-Lys-Beta-Ala-NH₂

A template composed of three residues of lysine was designed for the synthesis. First, an Fmoc-protected β-alanine residue, making up the C-terminus, was coupled to Rink amide resin. Then, this residue was deprotected and coupled to Fmoc-Lys(Fmoc)-OH with Fmoc-protected α- and ε-amino groups. Using the same protecting group for both amino groups allows for simultaneous deprotection, so that two amide bonds may be formed during the next coupling step, resulting in a branched peptide. A tetravalent core was achieved by repeating this step, in other words, both amino groups of this first lysine were coupled to lysine residues, once again protected with two Fmoc groups. Then, after deprotection, the first Fmoc-protected Arginine were coupled to the four NH₂ groups of the tri-lysine template, followed by additional Fmoc-protected Trp(tBoc)/Arg(Pbf), by using a PS3 automated solid phase peptide synthesizer (Protein Technologies, Inc.). Rink amide resin and all Fmoc-protected L-amino acids were purchased from NovaBiochem Corp. Crude peptide solutions were deprotected by 94% trifluoroacetic acid (TFA), 2.5% water, 2.5% triisopropylsilane(TIS), 1% ethanedithiol(EDT), precipitated in ether and purified on reverse phase HPLC. Molecular weights were verified by M/S using a Bruker MALDI-TOF spectrometer, [M+H]⁺ calcd 3380.9, found 3381.1.

Structure of dendrimeric antimicrobial peptide (Ac—WRWR)₄-lys₂-Lys-Beta-Ala-NH₂

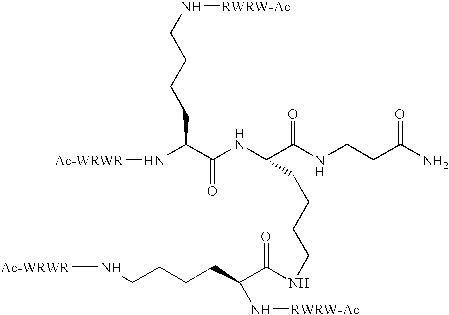

The methods for measuring antibacterial and hemolysis assays are identical to the methods used and described in Examples 1A and 1B. The results are shown below in Table 5.

Results

TABLE 5

| | ^{a}Antimicrobial | ^{a}Hemolytic | Hemolytic |
|---|---|---|---|
| | activity (μg/ml) | activity(μg/ml) | Index |
| Bacteria strain | IC₅₀ | HD₅₀ | HD₅₀/IC₅₀ |
| E. Coli (D31) | 9.5 | 137 | 14.4 |
| S Aureus (ATCC BAA-44) | 12.5 | 137 | 11.0 |
| B. anthracis (Sterne 34F2) | 15.5 | 137 | 8.8 |

^{a}The results are the mean of three independent experiments each performed in parallel.

In this work, our results give direct proof that multivalency of dendrimeric antimicrobial peptides also can increases their amphipathic structure, which in turn increases their activity with about ten fold improvement in IC₅₀ toward bacteria without provoking more severe hemolysis of red blood cells.

### Example 3

Length Effects in Antimicrobial Peptides: the Series (RW)ₙ

Native and synthetic antimicrobial peptides (AMP's) have been considered as a potential alternative source of new antibiotics (Boman, H. G. 2003. Antibacterial peptides: basic facts and emerging concepts. J Intern. Med. 254:197-215; Hancock, R. E., and D. S. Chapple. 1999. Peptide antibiotics. Antimicrob. Agents Chemother. 43:1317-23; Zasloff, M. 2002. Antimicrobial peptides of multicellular organisms. Nat. 415:389-95) for some time. As prospective antibiotics AMP's show a broad spectrum of activity against gram-negative and gram-positive bacteria including antibiotic-resistant bacterial strains, as well as some fungi, viruses, and parasites (Hancock, R. E. 1999. Host defence (cationic) peptides: what is their future clinical potential? Drugs. 57:469-73). Unfortunately, due to relatively high inhibitory concentrations, sensitivity to salts, and cytotoxic effects (Hancock, R. E. 1999. Host defence (cationic) peptides: what is their future clinical potential? Drugs. 57:469-73), their utility is currently limited to topical applications with the exception of injectable polymyxin (Brown, K. L., and R. E. W. Hancock. 2006. Cationic host defense (antimicrobial) peptides. Curr. Opin. Immunol. 18:24-30). A further obstacle to development of AMP's as therapeutics lies in the cost associated with manufacturing large quantities at competitive costs. Although some progress has been made in recombinant expression of fusion proteins comprising multiple copies of AMP's (Metlitskaia, L., Cabralda, J. E., Suleman, D., Kerry, C., Brinkman, J., D. Bartfeld, and M. M. Guama. 2004. Recombinant antimicrobial peptides efficiently produced using novel cloning and purification processes. Biotechnol. Appl. Biochem. 39:339-45), chemical peptide synthesis remains the preferred option for quality control purposes.

Known AMP's differ dramatically in size (12 to more than 50 amino acids), sequence and structure, sharing only amphipathicity and positive charge (Hancock, R. E. 1999. Host defence (cationic) peptides: what is their future clinical potential? Drugs. 57:469-73; Zasloff, M. 2002. Antimicrobial peptides of multicellular organisms. Nat. 415:389-95). This lack of sequence or structural homology makes it challenging to design potent synthetic antimicrobial peptides with desired activities or to predict the activity of peptides in vivo (Hancock, R. E., and D. S. Chapple. 1999. Peptide antibiotics. Antimicrob. Agents Chemother. 43:1317-23.). Comparison of AMP sequences reveals that two types of side chains are essential for antimicrobial activity. The cationic side chains arginine (R), lysine (K) or histidine (H) are thought to mediate peptide interactions with the negatively charged membranes and/or cell walls of bacteria, including lipopolysaccharide (Brown, K. L., and R. E. W. Hancock. 2006. Cationic host defense (antimicrobial) peptides. Curr. Opin. Immunol. 18:24-30). Bulky nonpolar side chains such as proline (P), phenylalanine (F), and tryptophan (W), occur frequently in AMP's, presumably providing lipophilic anchors that ultimately induce membrane disruption (51). Certain AMP's are evidently helix forming, while others favor beta structure. The role of conformation per se seems clear in helix forming peptides, but not universally (Deslouches, B., Phadke, S. M., Lazarevic, V., Cascio, M., Islam, K., R. C. Montelaro, and T. A. Mietzner. 2005. De novo generation of cationic antimicrobial peptides: influence of length and tryptophan substitution on antimicrobial activity. Antimicrob. Agents Chemother. 49:316-22 Deslouches, B., Phadke, S. M., Lazarevic, V., Cascio, M., Islam, K., R. C. Montelaro, and T. A. Mietzner. 2005. De novo generation of cationic antimicrobial peptides: influence of length and tryptophan substitution on antimicrobial activity. Antimicrob. Agents Chemother. 49:316-22; Houston, M. E., Kondejewski, Jr., L. H., Karunaratne, D. N., Gough, M., Fidai, S., R. S. Hodges, and R. E. Hancock. 1998. Influence of preformed alpha-helix and alpha-helix induction on the activity of cationic antimicrobial peptides. J. Pept. Res. 52:81-8). How peptides as short as dimers achieve membranolytic activity relative to longer membrane-spanning sequences remains unclear (Haug, B. E., Stensen, W., T. Stiberg, and J. S. Svendsen. 2004. Bulky nonproteinogenic amino acids permit the design of very small and effective cationic antibacterial peptides. J. Med. Chem. 47:4159-62). The effects of size, composition, and structure are not easy to deconvolute. One simplification of the problem is to investigate the effect of length of peptides with simple repeated sequences (Chen, P. W., C. L. Shyu, and F. C. Mao. 2003. Antibacterial activity of short hydrophobic and basic-rich peptides. Am. J. Vet. Res. 64:1088-1092) that contain balanced numbers of positively charged and hydrophobic side chains.

The side chains R and W appear in many AMP's that span a range of sizes and secondary structures. Lactoferricin B, indolicidin and tritrpticin (Bellamy, W., Takase, M., Wakabayashi, H., K. Kawase, and M. Tomita. 1992. Antibacterial spectrum of lactoferricin-B, A potent bactericidal peptide derived from the N-terminal region of bovine lactoferrin. J. Appl. Bacteriol. 73:472-479; Schibli, D. J., P. M. Hwang, and H. J. Vogel. 1999. Structure of the antimicrobial peptide tritrpticin bound to micelles: A distinct membrane-bound peptide fold. Biochem. 38:16749-16755; Selsted, M. E., Novotny, M. J., Morris, W. L., Tang, Y. Q., W. Smith, and J. S. Cullor. 1992. Indolicidin, A Novel Bactericidal Tridecapeptide Amide From Neutrophils. J. Biol. Chem. 267:4292-4295) are natural AMP's with sequences longer than 12 amino acids. PW2 and hexamers of sequence Ac—RRWWXX-NH₂ are active antibacterial peptides containing 6 to 12 amino acids derived from screening phage displays and synthetic combinatorial libraries, respectively (Blondelle, S. E., Takahashi, E., K. T. Dinh, and R. A. Houghten. 1995. The antimicrobial activity of hexapeptides derived from synthetic combinatorial libraries. J. Appl. Bacteriol. 78:39-46; Tinoco, L. W., da Silva, A., Leite, A., A. P. Valente, and F. C. L. Almeida. 2002. NMR structure of PW2 bound to SDS micelles. A tryptophan-rich anticoccidial peptide selected from phage display libraries. J. Biol. Chem. 277:36351-36356). Finally antimicrobial tetrapeptides and modified dipeptides containing R and W have been shown to retain antibacterial activities (Haug, B. E., Stensen, W., T. Stiberg, and J. S. Svendsen. 2004. Bulky nonproteinogenic amino acids permit the design of very small and effective cationic antibacterial peptides. J. Med. Chem. 47:4159-62; Strom, M. B., Haug, B. E., Skar, M. L., Stensen, W., T. Stiberg, and J. S. Svendsen. 2003. The pharmacophore of short cationic antibacterial peptides. J. Med. Chem. 46:1567-70).

However, no obvious pattern of R and W emerges from such a survey. The role of RW-rich motifs in AMP's has been investigated using quantitative structure activity relationships (QSAR) (Lejon, T., J. S. Svendsen, and B. E. Haug. 2002. Simple parameterization of non-proteinogenic amino acids for QSAR of antibacterial peptides. J. Pept. Sci. 8:302-6; Staubitz, P., Peschel, A., Nieuwenhuizen, W. F., Otto, M., Gotz, F., G. Jung, and R. W. Jack. 2001. Structure-function relationships in the tryptophan-rich, antimicrobial peptide indolicidin. J. Pept. Sci. 7:552-64; Strom, M. B., Haug, B. E., Rekdal, O., Skar, M. L., W. Stensen, and J. S. Svendsen. 2002. Important structural features of 15-residue lactoferricin derivatives and methods for improvement of antimicrobial activity. Biochem. Cell Biol. 80:65-74; Vogel, H. J., Schibli, D. J., Jing, W. G., Lohmeier-Vogel, E. M., R. F. Epand, and R. M. Epand. 2002. Towards a structure-function analysis of bovine lactoferricin and related tryptophan- and arginine-containing peptides. Biochem. Cell Biol. 80:49-63). Truncated peptide sequences from indolicidin and lactoferricin with conserved RW-motifs retain antimicrobial activity even when their original secondary structure is lost (Staubitz, P., Peschel, A., Nieuwenhuizen, W. F., Otto, M., Gotz, F., G. Jung, and R. W. Jack. 2001. Structure-function relationships in the tryptophan-rich, antimicrobial peptide indolicidin. J. Pept. Sci. 7:552-64; Vogel, H. J., Schibli, D. J., Jing, W. G., Lohmeier-Vogel, E. M., R. F. Epand, and R. M. Epand. 2002. Towards a structure-function analysis of bovine lactoferricin and related tryptophan- and arginine-containing peptides. Biochem. Cell Biol. 80:49-63), suggesting that R and W content alone correlates with activity. QSAR analysis of the antimicrobial activity of R— and W— peptides suggests that charge and multiple W— side chains are necessary, while W can be replaced by W-analogs with bulkier side chains (Lejon, T., J. S. Svendsen, and B. E. Haug. 2002. Simple parameterization of non-proteinogenic amino acids for QSAR of antibacterial peptides. J. Pept. Sci. 8:302-6; Strom, M. B., Haug, B. E., Rekdal, O., Skar, M. L., W. Stensen, and J. S. Svendsen. 2002. Important structural features of 15-residue lactoferricin derivatives and methods for improvement of antimicrobial activity. Biochem. Cell Biol. 80:65-74; Strom, M. B., O. Rekdal, and J. S. Svendsen. 2002. Antimicrobial activity of short arginine- and tryptophan-rich peptides. J. Pept. Sci. 8:431-7) The results also suggest that, in short peptides, the order of amino acids is less important than the overall composition with respect to cationic and lipophilic residues (Shafer, W. M., Hubalek, F., M. Huang, and J. Pohl. 1996. Bactericidal activity of a synthetic peptide (CG 117-136) of human lysosomal cathepsin G is dependent on arginine content. Infect. Immun. 64:4842-4845; Strom, M. B., Haug, B. E., Rekdal, O., Skar, M. L., W. Stensen, and J. S. Svendsen. 2002. Important structural features of 15-residue lactoferricin derivatives and methods for improvement of antimicrobial activity. Biochem. Cell Biol. 80:65-74; Strom, M. B., Haug, B. E., Skar, M. L., Stensen, W., T. Stiberg, and J. S. Svendsen. 2003. The pharmacophore of short cationic antibacterial peptides. J. Med. Chem. 46:1567-70).

Other studies suggest that antimicrobial activity of peptides containing R is higher than that of peptides containing K (Pellegrini, A., and R. von Fellenberg. 1999. Design of synthetic bactericidal peptides derived from the bactericidal domain P18-39 of aprotinin. BBA-Protein Struct. M. 1433:122-131; Shafer, W. M., Hubalek, F., M. Huang, and J. Pohl. 1996. Bactericidal activity of a synthetic peptide (CG 117-136) of human lysosomal cathepsin G is dependent on arginine content. Infect. Immun. 64:4842-4845). Similarly peptides containing W are more potent antimicrobials than those with either F or Y (Dathe, M., Nikolenko, H., J. Klose, and M. Bienert. 2004. Cyclization increases the antimicrobial activity and selectivity of arginine- and tryptophan-containing hexapeptides. Biochem. 43:9140-9150; Strom, M. B., Haug, B. E., Skar, M. L., Stensen, W., T. Stiberg, and J. S. Svendsen. 2003. The pharmacophore of short cationic antibacterial peptides. J. Med. Chem. 46:1567-70; Subbalakshmi, C., Bikshapathy, E., N. Sitaram, and R. Nagaraj. 2000. Antibacterial and hemolytic activities of single tryptophan analogs of indolicidin. Biochem. Biophys. Res. Commun. 274:714-716). The guanidinium group of R has a more dispersed positive charge than the single amine of K, possibly enhancing electrostatic interactions between peptides and the negatively charged bacterial membrane surface (Schibli, D. J., R. C. Montelaro, and H. J. Vogel. 2001. The membrane-proximal tryptophan-rich region of the HIV glycoprotein, gp41, forms a well-defined helix in dodecylphosphocholine micelles. Biochem. 40:9570-8; Vogel, H. J., Schibli, D. J., Jing, W. G., Lohmeier-Vogel, E. M., R. F. Epand, and R. M. Epand. 2002. Towards a structure-function analysis of bovine lactoferricin and related tryptophan- and arginine-containing peptides. Biochem. Cell Biol. 80:49-63). On the other hand the bulkier W-side chain may ensure more efficient interaction with membrane surfaces, allowing peptides to partition in the bilayer interface, in contrast with other non-polar side chains such as F, P or Y (Strom, M. B., Haug, B. E., Rekdal, O., Skar, M. L., W. Stensen, and J. S. Svendsen. 2002. Important structural features of 15-residue lactoferricin derivatives and methods for improvement of antimicrobial activity. Biochem. Cell Biol. 80:65-74; Wimley, W. C., and S. H. White. 1996. Experimentally determined hydrophobicity scale for proteins at membrane interfaces. Nat. Struct. Biol. 3:842-848). Recently, consideration of electrostatic effects including dipole and quadrupole moments of R and W side chains, respectively, suggest these properties may be involved in the ability to form hydrogen-bonds once peptides associate with membranes (Mavri, J., and H. J. Vogel. 1996. Ion pair formation of phosphorylated amino acids and lysine and arginine side chains: A theoretical study. Proteins: Struct., Funct., Genet. 24:495-501; Vogel, H. J., Schibli, D. J., Jing, W. G., Lohmeier-Vogel, E. M., R. F. Epand, and R. M. Epand. 2002. Towards a structure-function analysis of bovine lactoferricin and related tryptophan- and arginine-containing peptides. Biochem. Cell Biol. 80:49-63; Yau, W. M., Wimley, W. C., K. Gawrisch, and S. H. White. 1998. The preference of tryptophan for membrane interfaces. Biochem. 37:14713-14718).

While R and W play a role in many AMP's, the effects of chain length and composition on antimicrobial activity and selectivity have not been clearly distinguished. In this study a series of cationic peptides containing simple repeats, (RW)ₙ—NH₂ (n=1, 2, 3, 4 and 5) (Table 6) was synthesized, and we compared their antibacterial and hemolytic activities. Since natural peptides, such as indolicidin (Staubitz, P., Peschel, A., Nieuwenhuizen, W. F., Otto, M., Gotz, F., G. Jung, and R. W. Jack. 2001. Structure-function relationships in the tryptophan-rich, antimicrobial peptide indolicidin. J. Pept. Sci. 7:552-64) frequently have the C-terminal amidated, removing a negative charge that appears to lower activity, we used N-unprotected and C-terminal amide-protected peptides in this study. Selectivity was monitored by means of the hemolytic index, HI (also referred to as membranolytic selectivity index), defined as the ratio of hemolytic to antibacterial activity (Staubitz, P., Peschel, A., Nieuwenhuizen, W. F., Otto, M., Gotz, F., G. Jung, and R. W. Jack. 2001. Structure-function relationships in the tryptophan-rich, antimicrobial peptide indolicidin. J. Pept. Sci. 7:552-64; Tam, J. P., Y. A. Lu, and J. L. Yang. 2002. Antimicrobial dendrimeric peptides. Eur. J. Biochem. 269:923-32). Peptide conformation was investigated by CD spectroscopy. Interaction of peptides with two model lipids was examined using fluorescence.

Materials and Methods

Peptide Design and Synthesis.

The sequences (RW)ₙ—NH₂ (n=1-5) were assembled on Rink amide resin from Nova Biochem (San Diego, Calif.) with a RAININ Instrument PS3 solid phase synthesizer (Woburn, Mass.) using Fmoc chemistry. Fmoc-Trp(Boc)/Arg(pbf), the coupling reagent HBTU (2-(1H-benzotriazol-1-yl) 1,1,3,3-tetramethyluroniumhexafluoro phosphate) and HOBT (N-hydroxybenzotriazol) were also purchased from Nova Biochem. Cleavage of peptides from the resin was performed with 95% trifluoroacetic acid (TFA) in the presence of the scavenger 2.5% triisopropylsilane (TIS) and 2.5% H₂O. After precipitation with cold ether, samples were purified on a reverse-phase HPLC C-18 preparative column (2.2×25 cm, 300 Å, Grace Vydac Co., Hesperia, Calif.) with water and acetonitrile as eluents. Fractions containing product were pooled and lyophilized. The molecular weight of each peptide was confirmed by a Bruker matrix-assisted laser desorption/ionization time-of-flight (MALDI-TOF) mass spectrometer (Billerica, Mass.), giving the observed molecular weights shown in Table 6.

Antimicrobial and Hemolytic Assays.

The antimicrobial activity of each peptide was tested by following standard broth microdilution protocols recommended by the National Committee for Clinical Laboratory Standard (30). Ampicillin- and streptomycin-resistant strain E. coli (D31) and multi-drug resistant strain S. aureus (ATCC BAA-44) were obtained from the E. coli Genetic Resource Center (Yale University, New Haven, Conn.) and American Type Culture Collection (ATCC; Rockville, Md.), respectively. Bacteria were grown in Mueller Hinton Broth (MHB) at 37° C. for overnight. Then, cultures were diluted in MHB to a final concentration of 2×10⁴ to 2×10⁵ CFU/mL. Bacterial inoculums were incubated at 37° C. in PBS buffer (pH 7.2) with varying volumes of 2-fold dilution of peptide stocks. The 18-hour absorbance data were used to calculate the percent inhibition for each sample by comparing with the absorbance of cultures without peptides. The bacterial growth was measured by turbidity as optical density at 600 nm using a Genesys 5 Spectrophotometer (Rochester, N.Y.). All assays were carried out in triplicate. The concentration of peptide that resulted in 50% inhibition of growth was recorded as IC50.

Hemolytic activity of model peptides was assessed on fresh sheep erythrocytes (Fitzgerald Inc., Concord, Mass.). Peptide concentrations yielding 50% hemolysis were used as hemolytic dose (HD₅₀) determined from dose-response curves (Oren, Z., and Y. Shai. 1997. Selective lysis of bacteria but not mammalian cells by diastereomers of melittin: Structure-function study. Biochem. 36:1826-1835). The red blood cell suspension was incubated in PBS buffer (pH 7.2) with varying volumes of peptide stocks at 37° C. for 30 minutes, and then spun down at 3,000 rpm for 10 mins. The resulting supernatant was diluted by a factor of 40 in distilled water. The absorbances of the supernatant at λ=540 nm (OD₅₄₀) were measured in the UV Spectrophotometer. Zero hemolysis and 100% hemolysis controls were obtained by incubating the cells with buffer and 1% Triton-X, respectively. Hemolytic index (HI) was defined as HI=HD₅₀/IC₅₀.

Preparation of Liposomes.

Small unilamellar lipid vesicles (SUV) for fluorescence spectroscopy were prepared as described by Morrissey (29). Briefly, I-Palmitoyl-2-Oleoyl-sn-Glycero-3-[Phospho-rac-(1-glycerol)] (POPG) and 1-Palmitoyl-2-Oleoyl-sn-Glycero-3-Phosphocholine (POPC) lipids in chloroform were purchased from Avanti Polar Lipids (Alabaster, Ala.). Following chloroform evaporation, the POPG and POPC lipids were resuspended in 20 mM sodium phosphate buffer (pH 7.4) with 100 mM NaCl. The suspensions were vigorously vortexed and the samples were subsequently sonicated in an ultrasonic cleaner Branson B-220 (Danbury, Conn.) until the solution clarified.

CD Spectra.

Far UV CD spectra were recorded on an Aviv 202 CD spectrometer (Lakewood, N.J.) using 0.1 cm pathlength Hellma CD cuvettes (Forest Hills, N.Y.). The instrument was calibrated with (+)-10-camphorsulfonic acid standard purchased from Sigma-Aldrich Co. (St. Louis, Mo.). Spectra were recorded with 40 μM peptide in 20 mM phosphate buffer (pH 7.4, 100 mM NaCl) and 2 mM POPG or POPC SUV suspensions at 25° C. The concentration of the peptides was calibrated by UV absorbance of tryptophan residue at 280 nm. All CD spectra shown have the corresponding peptide-free solvent baselines subtracted. The results are expressed in terms of molar residue CD.

Peptide Binding to Lipids Measured by Tryptophan Fluorescence.

The tryptophan fluorescence spectra of the peptides were measured using an F-2500 fluorescence spectrophotometer from Hitachi High-Technologies America, Inc. (Chicago, Ill.). Tryptophan residues were excited at a wavelength of 295 nm and emission spectra were scanned from 300 to 450 nm using a scanning speed of 10 nm/s. Spectra were baseline corrected by subtracting blank spectra of the corresponding solutions without peptide. Experiments were carried out in 20 mM phosphate buffer (pH 7.4) with 100 mM NaCl. The concentration of peptides in all experiments was 10 μM calibrated by UV absorbance of tryptophan residue at 280 nm. Measurements were made in triplicate for each peptide in buffer and in the presence of each 500 μM POPG or POPC vesicles.

Fluorescence quenching experiments were conducted using acrylamide as a quenching reagent. The concentration of acrylamide was varied from 0.01 to 0.40M and the intensity of the spectra recorded. The peak maxima (F) were then compared to those recorded in the absence of quenching reagents (F₀). The effect of the quenching reagent on peptide fluorescence was compared by means of the quenching constant (K_{SV}) as determined by the Stern-Vollmer equation: F₀/F=1+K_{SV} [Q], where [Q] is the concentration of quencher.

Results

Comparison of Antimicrobial and Hemolytic Activities of the Peptides

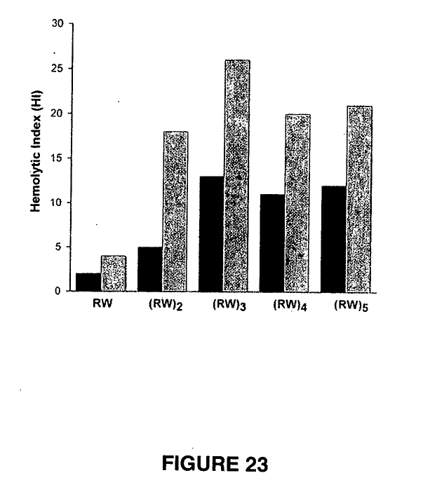

The calculated molecular weights and experimental determinations of the masses of the synthetic peptides indicate the products correspond to the designed sequences (Table 6). All five peptides show antimicrobial activity (Table 7). The di- and tetrapeptides are in agreement with data from Svendsen's group (Haug, B. E., Stensen, W., T. Stiberg, and J. S. Svendsen. 2004. Bulky nonproteinogenic amino acids permit the design of very small and effective cationic antibacterial peptides. J. Med. Chem. 47:4159-62; Strom, M. B., Haug, B. E., Skar, M. L., Stensen, W., T. Stiberg, and J. S. Svendsen. 2003. The pharmacophore of short cationic antibacterial peptides. J. Med. Chem. 46:1567-70). The chain length of the (RW)ₙ peptides strongly correlates with antibacterial activity: peptides with longer chains are much more effective in killing bacteria, but increasingly stimulate hemolytic activity. The hexamer, octamer and decamer are potent antimicrobial agents with IC₅₀ all roughly in μM range. Selectivity is measured by hemolytic index defined as the ratio of HD₅₀ to IC₅₀. FIG. 23 shows the hemolytic index as a function of chain length. While the longer chains are almost equally selective, hexamers offer an optimal choice in terms of efficiency of synthesis for both E. coli and S. aureus (FIG. 23).

Secondary Structures of the Peptides Studied by CD

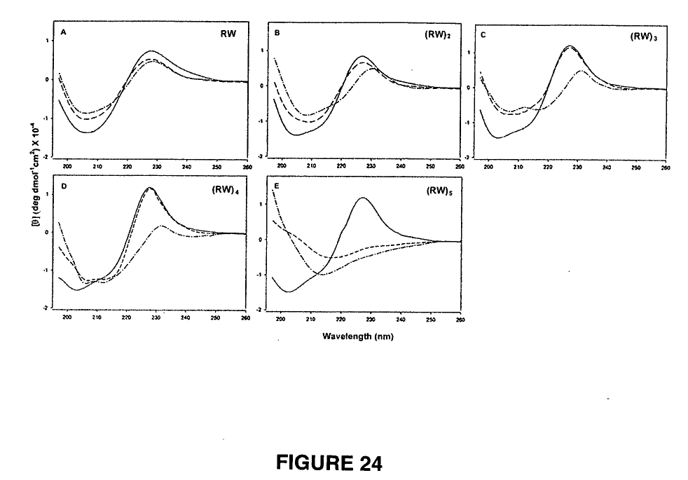

The CD spectra of the W-containing linear peptides (RW)ₙ dissolved in buffer show negative bands in the region between 200 and 210 nm and a positive band at 225-230 nm (FIG. 24). The negative band around 200 nm is characteristic of the small unfolded peptides, while the band at 225 nm is due to the indole side chain of W (Ladokhin, A. S., M. E. Selsted, and S. H. White. 1997. Bilayer interactions of indolicidin, a small antimicrobial peptide rich in tryptophan, proline, and basic amino acids. Biophys. J. 72:794-805; Woody, R. W. 1994. Contributions of tryptophan side-chains to the far-ultraviolet circular-dichroism of Proteins. Eur. Biophys. J. Biophy. 23:253-262), as seen in the CD spectrum of a peptide such as GGWGG containing a single chiral W residue (Shi, Z. S., Chen, K., Liu, Z., Ng, A., W. C. Bracken, and N. R. Kallenbach. 2005. Polyproline II propensities from GGXGG peptides reveal an anticorrelation with beta-sheet scales. Proc. Natl. Acad. Sci. U.S.A. 102:17964-17968). Each of the five peptides exhibits distinctive CD spectra in the presence of POPG and POPC. On binding a peptide to lipids, the decreased intensity at low wavelength implies loss of extended conformation. The apparent conformational changes are a function of chain length and lipid composition. For example, while (RW)₁ and (RW)₂ may be disordered in both buffer and lipid system, the CD of (RW)₃ shows ordering in the presence of POPG but not in POPC (FIG. 24).

Peptide Binding to Lipids Measured by Tryptophan Fluorescence Spectroscopy

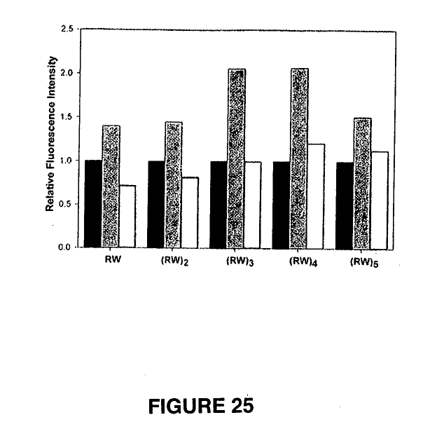

Fluorescence spectroscopy is a useful tool for monitoring Trp in different environments (Ladokhin, A. S. 1999. Evaluation of lipid exposure of tryptophan residues in membrane peptides and proteins. Anal. Biochem. 276:65-71). Interactions between the Trp in our model peptides and the vesicle lipids result in a blue-shifted in the emission spectrum following excitation at 295 nm. The shift to shorter wavelengths was observed to varying degrees for the fluorescence emission spectra of all (RW)ₙ peptides in the presence of phospholipids vesicles (Table 8). Additionally, an increase in emission intensity was also observed for the fluorescence emission when longer (RW)ₙ—NH₂ peptides (n≧3) bind to model lipids (FIG. 25). Observation of larger blue shifts and emission intensity in peptides binding to POPG suggest the Trp side-chain partitions preferentially into a more rigid, hydrophobic environment in POPG lipid bilayers, compared with those of POPC (Jing, W., Hunter, H. N., J. Hagel, and H. J. Vogel. 2003. The structure of the antimicrobial peptide Ac—RRWWRF—NH2 bound to micelles and its interactions with phospholipid bilayers. J. Pept. Res. 61:219-229).

The relative accessibility of Trp residues to solvent can be compared using Stern-Vollmer plots of the decrease in fluorescence as a function of an added soluble quencher. A decrease in quenching (smaller K_{SV}) reflects a more protected Trp residue. However it should be noted that Stern-Vollmer constants (K_{SV}), for longer peptides do not resolve individual Trp residues (Schibli, D. J., Epand, R. F., H. J. Vogel, and R. M. Epand. 2002. Tryptophan-rich antimicrobial peptides: comparative properties and membrane interactions. Biochem. Cell Biol. 80:667-677). Experimental results show that POPG and POPC offer the same protection to RW and (RW)₂ and therefore the same extent of Trp side-chain burial. However, increased protection in the presence of POPG vesicles is observed for longer (RW)ₙ (n≧3) peptides while the Trp residues of these peptides are buried more extensively in POPG than in POPC (Table 8). For example, the K_{SV} for (RW)₃ in aqueous solution is 18 M⁻¹ as compared to 1.8 and 2.8 M⁻¹ in POPG and POPC vesicles, respectively. These values indicate that the Trp side chains in free (RW)₃ are substantially more accessible to quencher than in the presence of lipids, and that (RW)₃ partitions more effectively into POPG vesicles than POPC vesicles.

Discussion

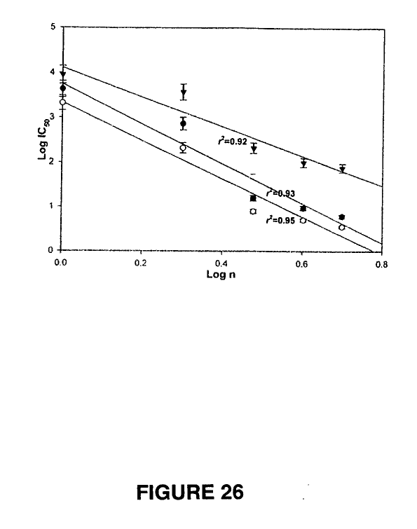

The study presented here demonstrates that longer chain linear peptides (RW)ₙ—NH₂ (n=1, 2, 3, 4 and 5) are more effective in killing both gram-negative and gram-positive bacteria. Interestingly the data fit a power law with respect to the chain length (log (IC₅₀)∞log(n)), with a slope of −4.5 (r²=0.93) and −4.3 (r²=0.95), for E. coli and S. aureus, respectively (FIG. 26). The longer chains also have increasing hemolytic activity on red blood cells with slope −3.3 (r²=0.92). (RW)₃—NH₂ was found to have the optimal selectivity for bacteria over blood cells relative to synthetic cost. CD spectroscopy is widely used to analyze the secondary structure of proteins and peptides because it is extremely sensitive to conformational changes. (RW)ₙ in buffer appears to be unfolded due to the negative band around 200 nm that is characteristic of unfolded model peptides (Shi, Z. S., Chen, K., Liu, Z., Ng, A., W. C. Bracken, and N. R. Kallenbach. 2005. Polyproline II propensities from GGXGG peptides reveal an anticorrelation with beta-sheet scales. Proc. Natl. Acad. Sci. U.S.A. 102:17964-17968); the positive band around 225 nm due to the indole side chain is Trp specific (Ladokhin, A. S., M. E. Selsted, and S. H. White. 1997. Bilayer interactions of indolicidin, a small antimicrobial peptide rich in tryptophan, proline, and basic amino acids. Biophys. J. 72:794-805; Woody, R. W. 1994. Contributions of tryptophan side-chains to the far-ultraviolet circular-dichroism of Proteins. Eur. Biophys. J. Biophy. 23:253-262). Peptide interactions with liposomes are accompanied by distinct CD spectral changes. Amphipathic AMP's such as indolicidin are thought to undergo an ordering transition on interaction with membranes (Ladokhin, A. S., M. E. Selsted, and S. H. White. 1999. CD spectra of indolicidin antimicrobial peptides suggest turns, not polyproline helix. Biochem. 38:12313-12319). However, it is hard to interpret the CD spectra simply in terms of secondary structure because of their Trp content. Changes in conformation are detected at shorter chain lengths in the presence of negatively charged POPG than in neutral POPC (FIG. 24). For example the CD spectrum of (RW)₃ indicates the presence of structure in POPG but remains disordered in POPC. This interpretation is supported by our NMR studies of (RW)₃ peptides conformation in negatively charged sodium dodecyl sulfate (SDS) and neutral dodecylphosphocholine (DPC) micelles. The structure ensemble of (RW)₃ peptides appears to be more ordered in SDS than in DPC (data not shown).

Trp-rich AMP's preferentially bind to model membranes containing a negatively charged headgroup relative to those containing a zwitterionic headgroup (Dathe, M., Nikolenko, H., J. Klose, and M. Bienert. 2004. Cyclization increases the antimicrobial activity and selectivity of arginine- and tryptophan-containing hexapeptides. Biochem. 43:9140-9150; Jing, W., Hunter, H. N., J. Hagel, and H. J. Vogel. 2003. The structure of the antimicrobial peptide Ac—RRWWRF—NH2 bound to micelles and its interactions with phospholipid bilayers. J. Pept. Res. 61:219-229; Schibli, D. J., Epand, R. F., H. J. Vogel, and R. M. Epand. 2002. Tryptophan-rich antimicrobial peptides: comparative properties and membrane interactions. Biochem. Cell Biol. 80:667-677). We recognize that the presence of multiple Trp residues does not allow for a simple interpretation of some of the spectroscopic data reported here (Schibli, D. J., Epand, R. F., H. J. Vogel, and R. M. Epand. 2002. Tryptophan-rich antimicrobial peptides: comparative properties and membrane interactions. Biochem. Cell Biol. 80:667-677). Our results are in accordance with previous studies, in which blue shifts and K_{SV} values (Table 8) demonstrate that the Trp residues in all five peptides partition to a greater extent in SUVs that contain negatively charged phosphatidylglycerol (POPG) (a major component of bacterial membranes) (Jing, W., Hunter, H. N., J. Hagel, and H. J. Vogel. 2003. The structure of the antimicrobial peptide Ac—RRWWRF—NH2 bound to micelles and its interactions with phospholipid bilayers. J. Pept. Res. 61:219-229). However there is no obvious trend in the blue shifts with respect to chain length, suggesting that the interactions with synthetic vesicles might imperfectly model those with intact bacterial membranes. Similarly even the shortest peptide is fully inaccessible to the quencher acrylamide in POPG. The picture from these experiments is that all the peptides of the series interact effectively with negatively charged POPG vesicles, a point we return to later in this discussion.

Membrane perturbation by cationic antimicrobial peptides has been proposed to proceed via membrane destabilization and/or transmembrane pore formation (Shai, Y. 2002. Mode of action of membrane active antimicrobial peptides. Biopolymers 66:236-48). Because of their short length it seems reasonable that (RW)ₙ peptides attack bacteria via membrane disruption rather than end-to-end pore assembly, although this is speculative. Arg residues provide electrostatic interactions that attract cationic peptides to negatively charged membrane surfaces (Juffer, A. H., C. M. Shepherd, and H. J. Vogel. 2001. Protein-membrane electrostatic interactions: Application of the Lekner summation technique. J. Chem. Phys. 114:1892-1905; Shepherd, C. M., Schaus, K. A., H. J. Vogel, and A. H. Juffer. 2001. Molecular dynamics study of peptide-bilayer adsorption. Biophys. J. 80:579-596). The indole ring of tryptophan in other RW rich AMP's (50) appears to partition into the interfacial region of membranes flanking the hydrophobic core (Hu, W., K. C. Lee, and T. A. Cross. 1993. Tryptophans in membrane proteins: indole ring orientations and functional implications in the gramicidin channel. Biochem. 32:7035-7047; Reithmeier, R. A. F. 1995. Characterization and modeling of membrane-proteins using sequence-analysis. Curr. Opin. Struct. Biol. 5:491-500; Wimley, W. C., and S. H. White. 1996. Experimentally determined hydrophobicity scale for proteins at membrane interfaces. Nat. Struct. Biol. 3:842-848). Linear RW chains might then interact preferentially with the polar headgroups, such that the peptides intercalate between the core leaflet of the bilayer and the polar headgroups. Peptides have been found to decrease the temperature of the main phase transition (Jing, W., Hunter, H. N., J. Hagel, and H. J. Vogel. 2003. The structure of the antimicrobial peptide Ac—RRWWRF—NH2 bound to micelles and its interactions with phospholipid bilayers. J. Pept. Res. 61:219-229) consistent with destabilization of the bilayer. Quenching studies with internal lipid spin labels report that related RW peptides associate with the hydrocarbon core of neutral bilayers and locate near the polar headgroups in negatively charged model membranes. Their precise position appears to be independent of peptide structure (Schibli, D. J., Epand, R. F., H. J. Vogel, and R. M. Epand. 2002. Tryptophan-rich antimicrobial peptides: comparative properties and membrane interactions. Biochem. Cell Biol. 80:667-677). The spectroscopic behavior of the (RW)ₙ peptides is consistent with an intercalation model (Aliste, M. P., and D. P. Tieleman. 2005. Computer simulation of partitioning of ten pentapeptides Ace-WLXLL at the cyclohexane/water and phospholipid/water interfaces. BMC Biochem. 6:30; Johnson, J. E., Rao, N. M., S. W. Hui, and R. B. Cornell. 1998. Conformation and lipid binding properties of four peptides derived from the membrane-binding domain of CTP: phosphocholine cytidylyltransferase. Biochem. 37:9509-9519). Intercalation of peptides into a membrane could indeed produce an increase in the lateral pressure near the interface leading to local disruption in the packing of lipid chains. The differences observed in the activity of (RW)ₙ could be associated with different spatial arrangements of the charged residues in the lipid head group region with increasing local density of RW motifs, resulting in different peptide surface areas and differently organized peptide-lipid clusters. A second point to consider is the extent to which bound peptides associate once they lie at the surface of the nonpolar core. Studies by Huang's group have shown clear evidence for concentration dependence in the interaction of peptides such as alamethicin with vesicles (6). A concentration dependent process in which effectively neutralized peptides associate more strongly with neighboring peptides as the chain length increases seems plausible, since the indole rings apparently do not bury in the core but orient along the interface (Wimley, W. C., Hristova, K., Ladokhin, A. S., Silvestro, L., P. H. Axelsen, and S. H. White. 1998. Folding of beta-sheet membrane proteins: A hydrophobic hexapeptide model. J. Mol. Biol. 277:1091-1110; Wimley, W. C., and S. H. White. 1996. Experimentally determined hydrophobicity scale for proteins at membrane interfaces. Nat. Struct. Biol. 3:842-848).

The CD spectra that reveal structural changes in peptides upon interaction with negatively charged bilayers can indeed be interpreted as evidence for intermolecular as well as intramolecular ordering. CD spectral changes are frequently observed when disordered peptides in solution partition into lipid bilayers (White, S. H., Wimley, W. C., A. S. Ladokhin, and K. Hristova. 1998. Protein folding in membranes: Determining energetics of peptide-bilayer interactions, Method Enzymol. 295:62-87). The high cost of partitioning peptide bonds into the membrane interface is a major driving force for secondary structure formation in membrane environments (Ladokhin, A. S., and S. H. White. 1999. Folding of amphipathic alpha-helices on membranes: Energetics of helix formation by melittin. J. Mol. Biol. 285:1363-1369; Wimley, W. C., Hristova, K., Ladokhin, A. S., Silvestro, L., P. H. Axelsen, and S. H. White. 1998. Folding of beta-sheet membrane proteins: A hydrophobic hexapeptide model. J. Mol. Biol. 277:1091-1110; Wimley, W. C., and S. H. White. 1996. Experimentally determined hydrophobicity scale for proteins at membrane interfaces. Nat. Struct. Biol. 3:842-848, 56). However for a peptide as short as RW formation of intermolecular structure seems more favorable than intramolecular structure. Thus we argue that the length effect reflects greater facility in inter-peptide interactions rather than exclusive formation of internal structure. How assembled peptides act to disrupt membranes remains unclear. In this work, we have identified (RW)₃ as a sequence with potential to serve as a relatively nontoxic antimicrobial that can be produced economically on a large scale.

TABLE 6
Amino acid sequences and molecular weights of (RW)ₙ—NH₂ peptides.

Molecular weight

| Sequence | Formula | Calcd | Obsd |
|---|---|---|---|
| RW—NH₂ | C₁₇H₂₅N₇O₂ | 359.4 | 359.4 |
| (RW)₂—NH₂ | C₃₄H₄₇N₁₃O₄ | 701.8 | 701.8 |
| (RW)₃—NH₂ | C₅₁H₆₉N₁₉O₆ | 1044.2 | 1044.3 |
| (RW)₄—NH₂ | C₆₈H₉₁N₂₅O₈ | 1386.6 | 1386.7 |
| (RW)₅—NH₂ | C₈₅H₁₁₃N₃₁O₁₀ | 1729.0 | 1729.1 |

TABLE 7
Antimicrobial and hemolytic activities of (RW)ₙ—NH₂ peptides.

| | |
|---|---|
| ^{a}IC₅₀, μM | ^{a,b}HD₅₀, μM |

| Peptides | E. coli | S. aureus | Red blood cell |
|---|---|---|---|
| RW—NH₂ | 4.3 × 10³ | 2.1 × 10³ | 8.9 × 10³ |
| (RW)₂—NH₂ | 7.3 × 10² | 2.1 × 10² | 3.7 × 10³ |
| (RW)₃—NH₂ | 16 | 8.0 | 2.1 × 10² |
| (RW)₄—NH₂ | 9.6 | 5.1 | 1.0 × 10² |
| (RW)₅—NH₂ | 6.2 | 3.6 | 76 |

^{a}The results are the mean of three independent experiments performed in parallel.
^{b}HD₅₀ determined from dose-response curve is peptide concentrations corresponding to 50% hemolysis.

TABLE 8
Fluorescence spectroscopy parameters measured for (RW)ₙ-NH₂ peptides
in the presence and absence of POPG and POPC vesicles.

| | λₘₐₓ | Blue shift (nm) | | ^{a}Kₛᵥ (M⁻¹) | | |
|---|---|---|---|---|---|---|
| Peptides | buffer(nm) | POPG | POPC | buffer | POPG | POPC |
| RW-NH₂ | 350 | 10 | 6.5 | 15 | 2.5 | 2.3 |
| (RW)₂-NH₂ | 350 | 11 | 6.0 | 14 | 2.7 | 2.5 |
| (RW)₃-NH₂ | 349 | 11 | 6.0 | 18 | 1.8 | 2.8 |
| (RW)₄-NH₂ | 350.5 | 10 | 6.0 | 17 | 2.3 | 5.8 |
| (RW)₅-NH₂ | 348 | 11 | 8.5 | 25 | 2.7 | 5.3 |

^{a}Stern-Vollmer constants Kₛᵥ (M⁻¹) were determined from the Stern-Vollmer equation: F₀/F = 1 + Kₛᵥ[Q], where [Q] is the concentration of quencher (acrylamide). Concentrations of quencher varied from 0.01 to 0.40M.

Summary

In summary, we have analyzed the relationship between the antibacterial activity of peptides in the series (RW)ₙ—NH₂ and their spectral properties in two model membrane systems. The results confirm that the RW combination is an active element for antimicrobial activity. Moreover, the antimicrobial activity of the peptides studied above increases with chain length, as does the hemolysis of red blood cells. Within the experimental error, longer peptides (n=3, 4 and 5) show similar values of the ratio between hemolytic and antibacterial activity, or hemolytic index. The hexamer (RW)₃ represents the optimal chain length in terms of efficacy of synthesis and selectivity as evaluated by the hemolytic index. The (RW)ₙ peptides show distinctive CD spectral changes upon interacting with model membranes. That is, CD spectroscopy indicates that these short peptides appear to be unfolded in aqueous solution but acquire structure in the presence of phospholipids. Interaction of the peptides with model lipid vesicles was examined using tryptophan fluorescence. The (RW)ₙ peptides preferentially interact with bilayers containing the negatively charged headgroup phosphatidylglycerol (POPG) relative to those containing a zwitterionic headgroup, phosphatidylcholine (POPC).

### Example 4

### The N-Terminal SRCR-SID Domain of gp340 Interacts with HIV-1 gp120 Sequences and Inhibits Viral Infection

Materials and Methods

Reagents

A complete set of gp120MN 15-mer peptides, overlapping by 11 amino acids, was obtained from NIH AIDS Reagent Program. SP6283, a SAG-binding sequence derived from HIV-1 gp120MN, was synthesized and HPLC purified to >95% purity by Sigma. All the viruses were obtained from NIH AIDS Reagent Program and propagated locally. 293/T or 293/O cells were obtained from ATCC. SwellGel™ nickel chelate discs were purchased from Pierce. Mabs 213-01 and 213-06 were from Antibody Shop (Copenhagen, Denmark) and GT-199 was obtained from GlycoTech (Rockville, Md.). Anti-HSV tag antibody was purchased from Novagen (Darmstadt, Germany). Polyclonal antibodies 1527 and 1529 and monoclonal antibodies 143 and 116 were raised to purified SAG (Wu Z W, Golub E, Abrams W R and Malamud D: gp340 (SAG) binds to the V3 sequence of gp120 important for chemokine receptor interaction. AIDS Res and Human Retroviruses 2004; 20:600-607, Takano K, Bogert M, Malamud D, Lally E and Hand A R: Differential distribution of salivary agglutinin and amylase in the Golgi apparatus and secretory granules of human salivary gland acinar cells. The Anatomical Record 1991; 230:307-318).

Expression of N-Terminal SRCR (V-SRCR)

PCR primers containing restriction sites at the 5′ ends spanning the leader sequence, first SRCR, and one half of the first SID were used on the DMBT1 plasmid. The resulting PCR product was digested and ligated into the TriEx 3 expression plasmid (Novagen, San Diego, Calif.) such that the C-terminal 6-his and HSV tags were in frame. The plasmid was sequenced and transfected into 293/O cells using the Fugene 6 reagent per manufacturer's instructions (Roche, Alameda, Calif.).

N-SRCR Protein Purification

N-SRCR/293 monolayers were rinsed with ice-cold PBS and lysed with ice-cold lysis buffer (10 mM Phosphate buffer pH 7.4, 1% Triton X-100, 0.1% SDS, 0.5% Na deoxycholate, 100 mM NaCl, 0.1% NaN3) containing a broad spectrum protease inhibitor cocktail (Complete Mini, Roche, Alameda, Calif.). The cells were disrupted by homogenization and insoluble debris removed by centrifugation at 1000×g for 10 min. The soluble fraction was batch mixed continuously at room temperature for 15 min with SwellGel™ nickel chelated discs (Pierce, Rockford, Ill.). Beads were transferred to a column and washed with 10× column volume of 50 mM Tris, pH 7.4-300 mM NaCl followed by step-wise 5× column volume aliquots of increasing concentrations of imidazole, ranging from 20 to 60 mM. The N-SRCR protein was eluted with 50 mM Tris, pH 7.4 containing 300 mM NaCl and 200 mM imidazole. Elution with 200 mM imidazole was repeated and the eluates pooled. The flow through (FT), washes, and eluted N-SRCR were analyzed by SDS-PAGE and Western blots probed with either a gp-340-specific polyclonal antibody or a monoclonal antibody specific for the HSV tag. The pooled eluates were dialyzed against 10 mM Tris, pH7.4 and concentrated by lyophilization.

SDS-PAGE and Western Blot:

SDS-PAGE and Western blot analyses were performed as reported 4. When indicated, Protein samples were reduced with 50 mM DTT in the presence of 0.1% SDS at 100° C. for 5 min. The antibodies used were protein-A column purified rabbit polyclonal antibody 1527 and 1529 (used at 1000× dilution), gp-340-specific monoclonal antibody 213-06 and a monoclonal antibody specific for HSV gD tag used at a concentration of 0.4 μg/ml (Novagen, San Diego, Calif.).

Peptide Binding Assay (Solid Phase ELISA

Solid phase ELISA for N-SRCR-peptide interaction was performed as reported 1 with modifications. Briefly, 96-well, Ubottom vinyl plates were coated with 2.5 μg peptides/well in 50 μl of sodium bicarbonate buffer, pH 9.6, at 4° C., overnight. The plates were washed four times by flooding and emptying the plate with Washing Buffer containing 20 mM Tris-HCl, pH 7.4, 0.05% Tween-20, 4 mM CaCl₂ prior to blocking with 100 μl/well of 2% blotting grade non-fat milk (Bio-Rad Laboratories, Hercules, Calif.) at 37° C. for 60 min. SAG purified from saliva, recombinant gp-340, or N-SRCR was diluted in the Binding Buffer (20 mM Tris-HCl, pH 7.4, 4 mM Ca++ and 0.05% Triton X-100) and incubated with the solid-phase gp-120 derived 15-mer peptides at 37° C. for 60 min. The bound ligand was detected with protein A-purified 1527 antibody and APconjugated goat anti-rabbit antibody and ImmunoPure PNPP substrate (Pierce Biotechnology Inc., Rockford, Ill.) in DEA buffer measured at 410 nm.

HIV Inhibition Assay

The inhibition assay was carried out as previously reported 4. Briefly, virus was pre-treated at 37° C. for 60 min with purified N-SRCR in serial dilutions of RPMI-1640 containing 10% FCS, 1 mM CaCl2 and 2 ug/ml polybrene. The highest SRCR concentration utilized was always below 15 μg/ml, which was the pre-determined toxic dosage. Either 1×10⁵ IL-2 stimulated primary human PBMCs or 5×10⁴H9 cells were added and the infection carried out at 37° C. for 18 hours before the excess virus and the inhibitors were removed by 4× washings with culture medium. The cells were re-fed with medium containing the same concentrations of N-SRCR. Culture supernatant p24 levels were determined after 5-6 days of infection, using a commercial assay (Beckman-Coulter, Fullerton, Calif.).

Viral Attachment Assay

0.5×10⁶ H9 cells were pre-incubated with purified gp-340 ranging from 2 to 10 μg/ml, or N-SRCR at 0.5 μg/ml at 37° C. for 60 min before viral exposure at 50 ng/ml p24 at 37° C. for 3 hours. The exposed cells were washed repeatedly to remove unbound virus, either lysed for cell-associated viral p24 measurement, treated with trypsin for 5 min before lysis, or refed with culture medium and maintained in culture for an additional 4 days. Culture medium was collected, cleared of cellular debris, lysed and viral p24 was measured.

Results

Expression and Purification of SRCR

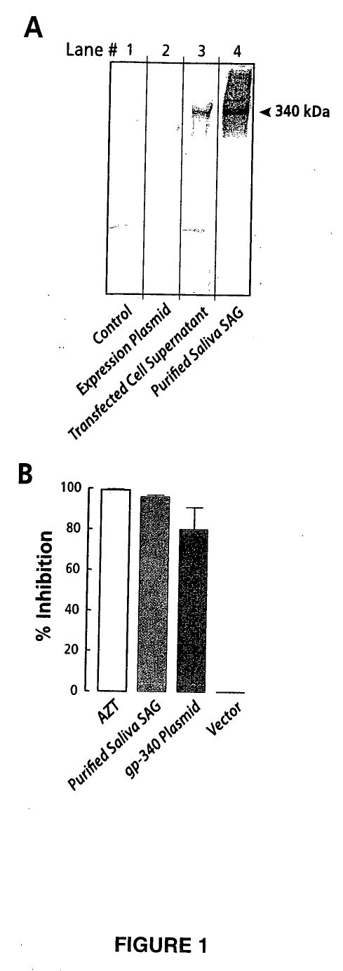

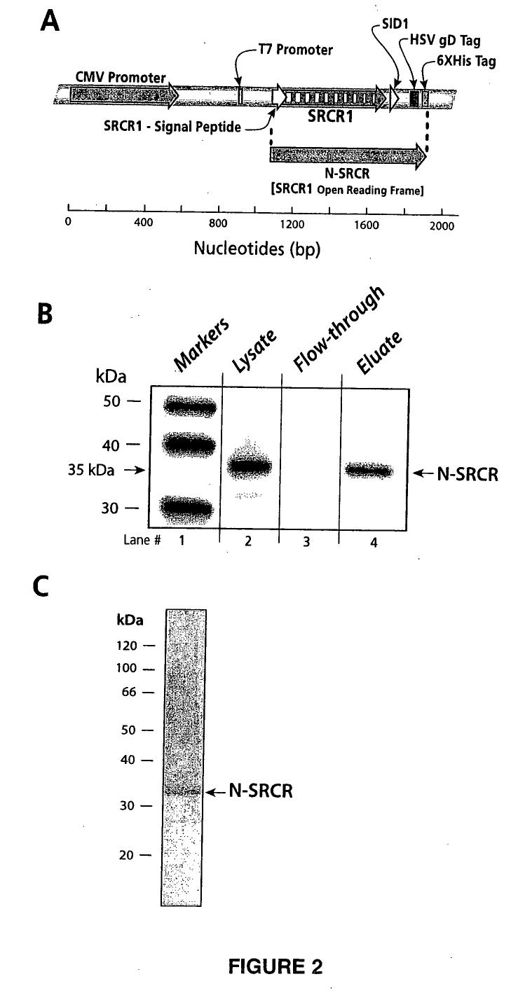

An expression plasmid containing the full-length gp-340 gene in the pDEST 12.2 vector was obtained from Holmskov 5. It contained a CMV promoter and a neomycin selection marker. The transfected 293T cells secreted a protein that co-migrated with SAG on SDS-PAGE and was reactive with a SAG-specific antibody, Ab-1527, on Western (FIG. 1A). In an inhibitory assay, this protein exhibited potent anti-HIV-1 activity compared to SAG purified from human saliva (see FIG. 1B). To determine the functional roles of SRCR domains of gp-340 in inhibiting HIV-1 infection, a construct containing the N-terminal sequence, starting from the signal peptide to halfway through the first SID sequence, was cloned into the pTriEx 3 expression vector containing a CMV promoter and a G418 selection marker. To assist in purification, two tags were included downstream to the N-SRCR gene at the Cterminus (see FIG. 2A), an HSV gD sequence and a His-6 tag. Transfectants were selected in G418 at an early time point and high producing stable clones were generated by limiting dilution cloning and screened by SDS gel electrophoresis and Western blots, probed with a polyclonal anti-serum raised against SAG, Ab-1527, or a commercial Mab against the HSV tag, anti-gD.

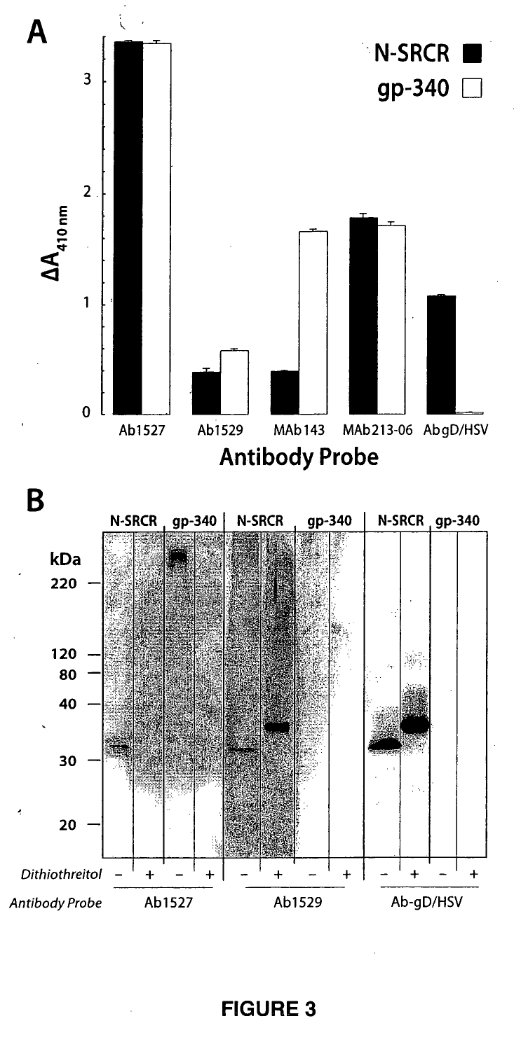

The expressed protein was only minimally secreted into the culture supernatant and most of the protein was retained intracellularly (data not shown). Therefore, the protein was isolated from cell lysates, and purified by taking advantage of the His tag using affinity metal ion chromatography. The purification of the protein was monitored by Western blots (FIG. 2B) and the protein purity assessed by Coomassie Blue staining. As shown, the HSV gD specific monoclonal antibody detected a protein with an apparent molecular weight of 35 kDa, (FIG. 2B) that corresponded to a Coomassie Blue-stained band with the same molecular weight (FIG. 2C). Characterization of the purified protein with a panel of monoclonal antibodies (FIG. 3) showed that N-SRCR was reactive with an antibody specific for a SAG (gp-340) conformational epitope detected with Mab 143, polyclonal antibody Ab-1527 raised against native SAG and Ab-1529 raised against denatured SAG. The expressed protein was also recognized by Mab 213-06, a commercial antibody raised against gp-340 purified from bronchioalveolar lavage and recognizing a conformational epitope (Bikker F J, Ligtenberg A J, van der Wal J E, et al.: Immunohistochemical detection of salivary agglutininlgp-340 in human parotid, submandibular, and labial salivary glands. J Dent Res 2002; 81: 134-139 and unpublished data]. Little reactivity was detected with antibodies specific for sialic acid (data not shown), known to be present on intact gp-340. Polyclonal Ab1527 did not bind to N-SRCR recombinant protein after reduction with DTT (FIG. 3B), a property also seen with intact gp-340. In contrast, anti-gD Mab and polyclonal Ab 1529 retained their reactivity with the reduced protein. The apparent decreased mobility of N-SRCR after reduction reflected the disruption of the disulfide bonds in the protein.

N-SRCR Protein Interaction with gp120 and a V3 Peptide

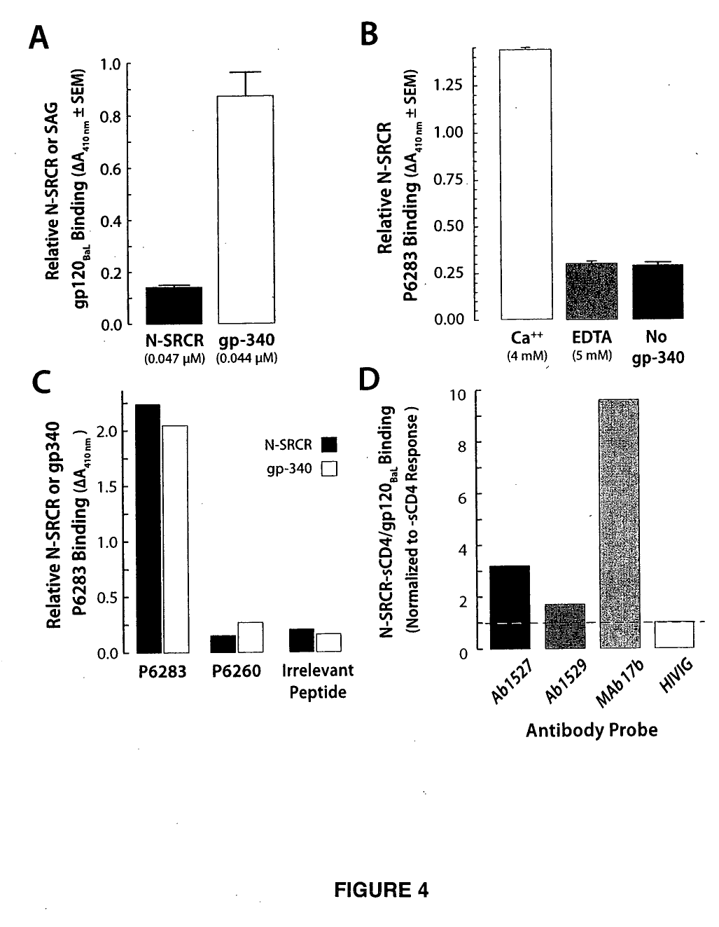

Purified N-SRCR protein was tested in an ELISA for interaction with gp120 captured via its N-terminus with antibody D7324 and shown to bind immobilized gp120. At an equal molar concentration, N-SRCR bound to gp120 significantly less than to gp-340 (FIG. 4A). In an earlier study, we demonstrated that SAG (gp-340) bound to a peptide sequence (P6283) derived from the N-terminus of the gp120MN V3 loop in a Ca++-dependent manner 1. EDTA also blocks the interaction of N-SRCR with P6283 (FIG. 4B). However, binding of N-SRCR to the peptide P6283 is comparable to binding of gp-340 (FIG. 4C), in contrast to the enhanced binding of gp-340 to the entire gp120 molecule (FIG. 4A).

The N-SRCR interaction with gp120 was enhanced by sCD4 pre-binding to gp120. As shown in FIG. 4D, N-SRCR binding to gp120 was significantly increased upon sCD4 treatment of gp120, as detected by Ab-1527. The sCD4-induced conformational shift was evident, as indicated by a dramatically increased 17b binding to gp120 (FIG. 4D). The HIVIG reactivity indicated that the amount of gp120 remained constant after incubation with sCD4. The observation is consistent with that seen for SAG binding to gp120 1, suggesting that N-SRCR also exhibits enhanced binding to gp120 following exposure to sCD4.

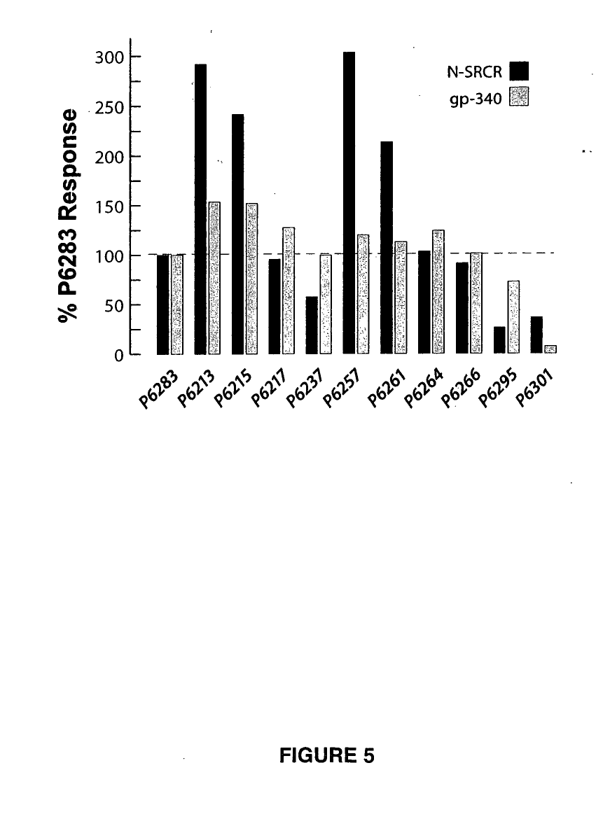

To investigate if gp-340 or N-SRCR protein interacts with other regions of gp120, both proteins were analyzed for their binding to a set of overlapping synthetic peptides derived from the complete env sequence of HIV-1 MN (NIH AIDS Reagent Program). In addition to the V3 peptide (P6283), SAG interacted with 8 peptides from other regions of the gp120 molecule (FIG. 5). The N-SRCR protein reacted most strongly with the same peptides as the entire gp-340. All the peptide binding to either gp-340 or N-SRCR required the presence of Ca++ (data not shown). Interestingly, a number of peptides (P6213 and P6215) reacted with both N-SRCR and gp-340 at higher levels than P6283, while P6295, P6301 showed lower binding. N-SRCR generally exhibited relatively higher reactivity with P6213, P6215 and P6261 but lower reactivity with P6237, and P6295 as compared to gp-340. The binding of either gp-340 or N-SRCR to other gp120 derived peptides was not significantly above the blank (data not shown).

N-SRCR Interaction with the V3 Peptide is Likely Mediated by SRCR Domain

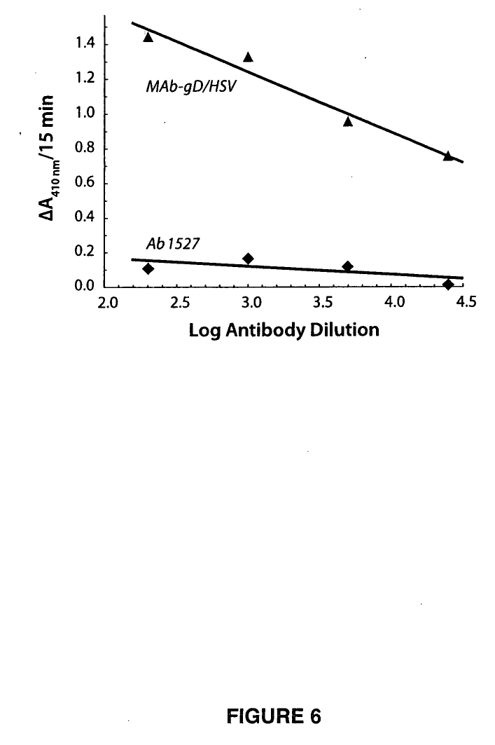

A number of antibodies were evaluated for recognition of N-SRCR protein bound to the V3 peptide P6283. Polyclonal Ab1527 has significantly reduced binding to N-SRCR protein that is bound to immobilized P6283, the HIV-1 gp120 peptide derived from the V3 sequence of MN strain. However, Ab1527 reacts strongly with N-SRCR when it is immobilized directly on the plate (see FIG. 3A). Antibody to the HSV/gD epitope demonstrates strong reactivity with to peptide-bound recombinant N-SRCR. These contrasting binding characteristics are illustrated as titrations against N-SRCR protein bound to P6283 (FIG. 6). These data suggests that the interaction of N-SRCR protein and the HIV-1 V3 peptide P6283 occludes Ab1527 binding epitopes.

N-SRCR Inhibition of HIV-1 Infection

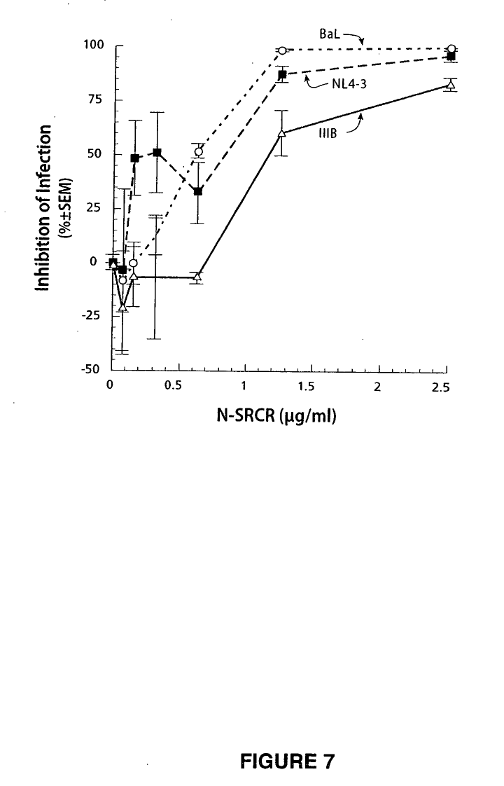

Purified N-SRCR was evaluated for anti-HIV-1 activity in an inhibitory assay using either stimulated human PBMCs or H9 cells. Three viral isolates representing both CCR5- and CXCR4-using phenotypes were assayed. FIG. 7 shows that two CXCR4 and one CCR5-using viruses were inhibited by N-SRCR. The estimated ID90 for NL4-3 was 1.5 μg/ml and 1.1 μg/ml for BaL. An ID90 for IIIB was not achieved, however the ID75 was 2 μg/ml. N-SRCR was not toxic to H9 cells at concentrations up to 10 μg/ml.

Effect of gp-340 or N-SRCR on HIV-1 Attachment to Target Cells

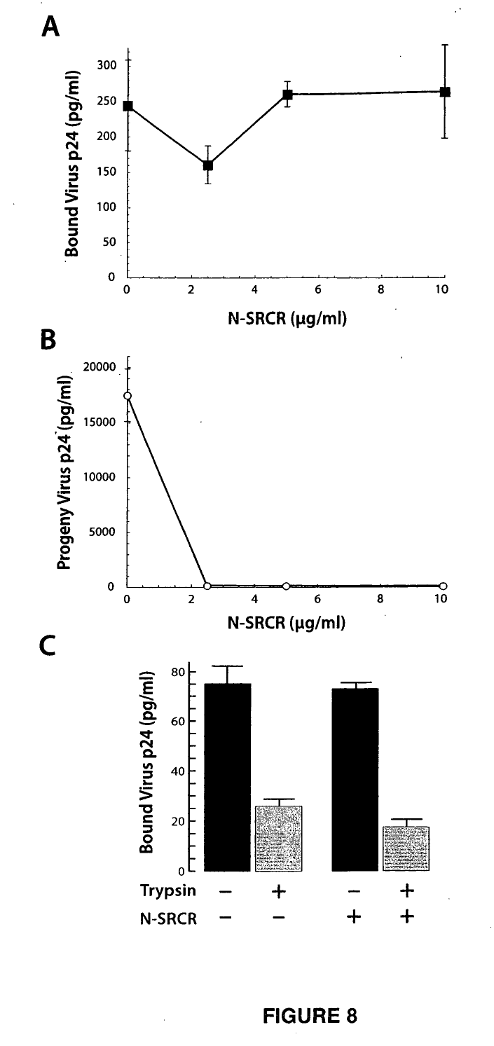

Our earlier data demonstrated that sCD4 binding to gp120 had no effect on subsequent gp-340 binding 4 suggesting that gp-340 binds to a distinct region on gp120 and that gp-340 inhibits viral infection at a post-binding step. This hypothesis was substantiated by identifying gp-340 interaction with a V3 N-terminal sequence that is also important for virus-co-receptor interaction. To further explore the mechanism of gp-340 inhibition, the effect of this inhibition on HIV-1 binding to target cells was studied. Gp-340-treated virus was incubated with target cells for 4 hours, washed and the cells were lysed to measure bound virus. FIG. 8A shows that the cell-associated virus was not affected by exposure to gp-340. In contrast, the viral replication in the treated cells was significantly inhibited (FIG. 8B). Similar results were obtained in the SRCR-treated cells, as shown in FIG. 8C. The N-SRCR treatment also did not effect viral attachment. When cells were treated with trypsin following viral challenge, the cells treated with or without N-SRCR showed no significant difference in cell-associated virus. These observations are consistent with earlier studies and the effect of sCD4-enhanced N-SRCR binding, and suggests that gp-340 and N-SRCR inhibit HIV-1 infection by blocking a step post-host cell attachment.

Discussion

The lung scavenger receptor protein gp-340 binds to surfactant proteins D (SP-D) (Holmskov U, Mollenhauer J, Madsen J, et al.: Cloning of gp340, a putative opsonin for surfactant protein D. Proc. Natl. Acad. Sci. 1999; 96:10794-10799) and probably SP-A as an opsonin receptor believed to play important roles in host defense against pathogens. Salivary agglutinin (SAG), which is encoded by the DMBT-1 gene, has been implicated in the oral clearance of microorganisms and innate protection against dental caries (Ericson T and Rundegren J: Characterization of a salivary agglutinin reacting with a serotype c strain of Streptococcus mutans. Eur. J. Biochem. 1975; 133:255-261; Demuth DR, Golub EE and Malamud D: Streptococcal-host intractions: Structural and functional analysis of a S. sanguis receptor for a human salivary glycoprotein. J Biol Chem 1990; 265:7120-7126; Carlen A, Bratt P, Stenudd C, Olsson J and Stromberg N J: Agglutinin and acidic proline-rich protein receptor patterns may modulate bacterial adherence and colonization on tooth surfaces. Dent Res. 1998; 77:81-90). Our earlier studies extended the understanding of this innate immune component by demonstrating that gp-340/SAG possesses potent anti-HIV-1 activity, (Nagashunmugam T, Friedman H M, Davis C, Kennedy S, Goldstein L T and Malamud D: Human submandibular saliva specifically inhibits HIV-1. AIDS Res. Human Retro. 13 1997; 371-376) and gp-340 inhibited virus infection by directly binding to the virus envelop gp120 glycoprotein in a Ca++ dependent manner (Wu Z W, Van Ryke D, Davis C, et al.: Salivary agglutinin (SAG) inhibits HIV-1 infectivity through interaction with the envelope glycoprotein. AIDS Res and Human Retroviruses 2003; 19:201-209). Recently, we determined that gp-340/SAG interacted with a linear sequence derived from a highly conserved region in the N-terminal stem of HIV-1 V3 loop and this interaction required Ca++ and was enhanced by pre-binding of sCD4 (Wu Z W, Golub E, Abrams W R and Malamud D: gp340 (SAG) binds to the V3 sequence of gp120 important for chemokine receptor interaction. AIDS Res and Human Retroviruses 2004; 20:600-607), a process that is believed to be important for HIV-1 co-receptor binding (Sattentau Q J and Moore J P: Conformational changes induced in the human immunodeficiency virus envelope glycoprotein by soluble CD4 binding. J. Exp. Med 1991; 174:407-415; Trkola A, Dragic T, Arthos J, et al.: CD4-dependent, antibody-sensitive interactions between HIV-1 and its co-receptor CCR-5. Nature 1996; 384:184-187; Wu L, Gerard N P, Wyatt R, et al.: CD4-induced interaction of primary HIV-1 gp120 glycoproteins with the chemokine receptor CCR-5. Nature 1996; 384:179-183). We postulated that the anti-viral activity of gp-340/SAG is mediated by binding to this sequence and thus blocking the virus access to co-receptors (Wu Z W, Golub E, Abrams W R and Malamud D: gp340 (SAG) binds to the V3 sequence of gp120 important for chemokine receptor interaction. AIDS Res and Human Retroviruses 2004; 20:600-607). However, it was not known what regions of gp-340/SAG were involved in binding to gp120. Studies on SAG-bacteria interactions have shown that carbohydrate structure on gp-340 play only a partial role in binding and agglutination of bacteria (Demuth DR, Golub EE and Malamud D: Streptococcal-host intractions: Structural and functional analysis of a S. sanguis receptor for a human salivary glycoprotein. J Biol Chem 1990; 265:7120-7126). More recently, Bikker et. al., (Bikker F J, Ligtenberg A J, van der Wal J E, et al.: Immunohistochemical detection of salivary agglutinin/gp-340 in human parotid, submandibular, and labial salivary glands. J Dent Res 2002; 81:134-139; Bikker F J, Ligtenberg A J M, End C, et al.: Bacteria binding by DMBT1/SAG/gp-340 is confined to the VEVLXXXXW motif in its scavenger receptor cysteinerich domains. J Biol. Chem. 2004; 279:47699-47703) reported that a 16-mer linear peptide derived from the consensus SRCR sequence was able to agglutinate S. mutans, and to bind to a wide variety of Gram-positive and Gram-negative bacteria. The study suggested possible roles of SRCR domain in bacterial aggregation. In the present study, we demonstrate the direct N-SRCR interaction with HIV-1 gp120 sequences and the anti-viral activity of N-SRCR protein. To our knowledge, this is the first report showing the direct involvement of the N-terminal SRCR region in binding HIV-1 env and mediating anti-viral activity.

Characterization with antibodies indicated that the N-SRCR protein expressed many of the epitopes recognized by antibodies raised against intact gp-340 (FIG. 3). The N-SRCR protein also shared a similar structural property with gp-340, as indicated by three antibodies specific for conformational epitopes (Mab 143, Mab 213-06 and polyclonal antibody Ab1527). All evidence suggested that the expressed protein resembles the SRCR domain of gp-340 and the protein appears to be correctly folded. Although the N-SRCR was engineered to be secreted from the cell, it was retained intracellularly. Sequence analysis predicted that there was a 50% chance that the recombinant N-SRCR protein could be retained in inclusion bodies due to a high percentage of hydrophobic amino acid residues. An alternative but unlikely explanation is that the signal peptide was prematurely truncated during synthesis.

The peptide binding studies showed that the N-SRCR protein reacted with the same V3 peptide as gp-340 and SAG and that this interaction is Ca++-dependent (FIGS. 4B and 4C). The N-SRCR-peptide interaction is likely mediated by the N-SRCR domain rather than the tags since the gp-340-specific anti-serum 1527 reacted poorly with the N-SRCR protein when bound to P6283. In contrast, the HSV-specific antibody reacted with the bound N-SRCR-SID protein (FIG. 6), suggesting that 1527 epitopes were blocked by the N-SRCR-peptide interaction and the HSV sequence adjacent downstream to the SID was away from the interaction. Since polyclonal antibody 1527 recognizes epitope(s) highly sensitive to disulfide reduction and the non-SRCR N-terminus and the SID sequence contain no disulfide bonds, it is reasonable to assume that Ab 1527 epitope(s) are located within the SRCR domain. However, it cannot be completely ruled out that the N-terminus non-SRCR or the SID sequence may play a role in gp120 binding and anti-viral activity.

The multiple gp120 binding sites of either N-SRCR or gp-340 and the requirement of Ca++ are intriguing. Analysis has shown that the reactive peptides in gp120 do not share common sequence motif (data not shown). However, a crystal structure of variable loop-deleted gp120 complexed with a two-domain CD4 and Mab 17b suggests that the regions represented by the reactive peptides are brought into proximity and the regions are involved in co-receptor binding (Zolla-Pazner S: Identifying epitopes of HIV-1 that induce protective antibodies. Nat Rev Immunol 2004; 4:199-210). Our structural modeling stitched with an NMR-derived V3 structure (Wu Z W, Golub E, Abrams W R and Malamud D: gp340 (SAG) binds to the V3 sequence of gp120 important for chemokine receptor interaction. AIDS Res and Human Retroviruses 2004; 20:600-607) also predicts that the V3 stem sequence (P6283) is located on the same face as the other reacting peptides (unpublished data). Various gp120 regions may bind multiple gp-340 molecules through interacting with the SRCR-1 domain or alternatively the SRCR-1 domain from which N-SRCR was derived binds the V3 N-terminal sequence while other SRCR domains of gp-340 interact with other gp120 sequences. It is possible that gp-340 interacts with multiple sequences of gp120 to increase binding affinity or avidity. The binding of N-SRCR and gp-340 at an equal molar concentration suggested that multiple SRCR domains on gp-340 mediated such an interaction. Kinetic studies and competition analysis will be needed to definitively address the issue.

Ca++ may either be directly involved in protein-protein interaction or indirectly influence the conformational structure of N-SRCR or gp-340. Based on the Ca++requirement for N-SRCR binding to all reactive gp120 sequences, it is likely that Ca++ is involved in maintaining gp-340 or N-SRCR structure. Structural analysis of N-SRCR or gp-340 with or without Ca++ is being performed to address the issue.

The specificity of anti-HIV-1 inhibitory activity of N-SRCR is similar to gp-340/SAG since both CCR5 and CXCR4 viruses were inhibited with comparable potency. This property can be explained by the fact that N-SRCR protein binds to a highly conserved viral sequence that may be involved in co-receptor binding. We hypothesize that both gp-340 and N-SRCR inhibited HIV-1 infection by blocking virus binding to its co-receptor. This hypothesis is substantiated by the observation that neither N-SRCR nor gp-340 affected virus binding to target cells but viral infection was reduced, suggesting that the blocking is at a post CD4 binding step.

In summary, a truncation fragment representing the N-terminal region of the DMBT-1 gene product including the first SRCR and the first half of the SID sequence was successfully expressed and the anti-HIV-1 activity was confirmed. The expressed N-SRCR protein shared many properties similar to its parental gp-340 and exhibited potent anti-HIV-1 activity. The mode of the action, its potency against both CXCR4 and CCR5 viruses, and the small size of the molecule make N-SRCR an interesting candidate as a therapeutic target or as a potential microbicide for blocking HIV-1 transmission.

### Example 5

### Expression of Full Length gp340 and its Ability to Block HIV-1 Infection: Comparison with SAG

Earlier studies identified salivary agglutinin (SAG) a 340 kDa glycoprotein previously characterized as an agglutinin for oral bacteria (Demuth D R, Larnmey M S, Huck M, Lally E T and Malamud D: Comparison of Streptococcus mutans and Streptococcus sanguis receptors for human salivary agglutinin. Microb Pathog 1990; 9: 199-211) as the major HIV inhibitor present in human saliva (Wu Z W, Van Ryke D, Davis C, et al.: Salivary agglutinin (SAG) inhibits HIV-1 infectivity through interaction with the envelope glycoprotein. AIDS Res and Human Retroviruses 2003; 19:201-209). In a series of anti-viral studies we described this activity as HIV-1 specific (Nagashunmugam, Malamud, D., Davis, C., Abrams, W. R and Friedman, H. M. (1998). Human submandibular saliva inhibits human immunodeficiency virus type 1 infection by displacing envelope glycoprotein gp120 from the virus. J Infect Dis 178(6): 1635-1641; Wu Z W, Van Ryke D, Davis C, et al.: Salivary agglutinin (SAG) inhibits HIV-1 infectivity through interaction with the envelope glycoprotein. AIDS Res and Human Retroviruses 2003; 19:201-209), and reported that its mechanism of action involved binding to gp120 (Wu Z W, Golub E, Abrams W R and Malamud D: gp340 (SAG) binds to the V3 sequence of gp120 important for chemokine receptor interaction. AIDS Res and Human Retroviruses 2004; 20:600-607). Subsequently, SAG was identified by proteomic analysis as gp340, an alternatively spliced form of DMBT1, a member of the SRCR family of proteins (Prakobphol A, Feng X, Hoang V M, et al.: Salivary agglutinin, which binds to S. mutans and H. pylori, is the lung scavenger receptor cysteine-rich protein gp340. J. Biol. Chem. 2000; 275:39860-39866).

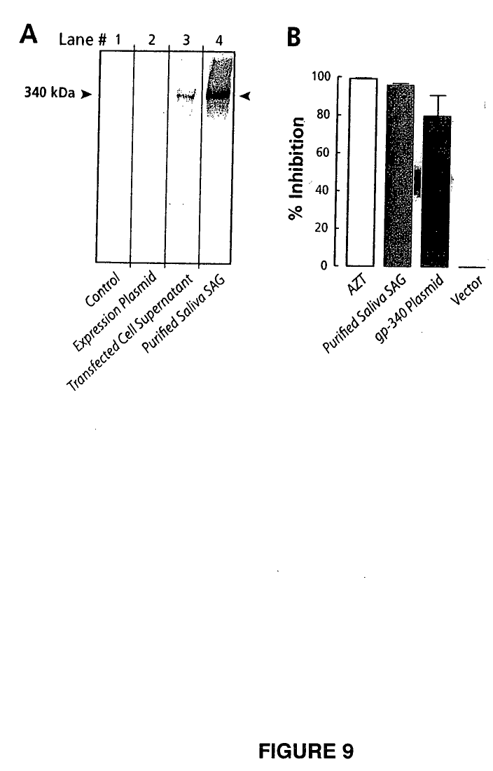

We obtained an expression plasmid containing the full length gp340 gene in the pDEST 12.2 vector, transfected mammalian 293 cells with the plasmid, and demonstrated that the expressed glycoprotein was as active in blocking HIV-1 infection as SAG purified from human saliva (FIG. 9).

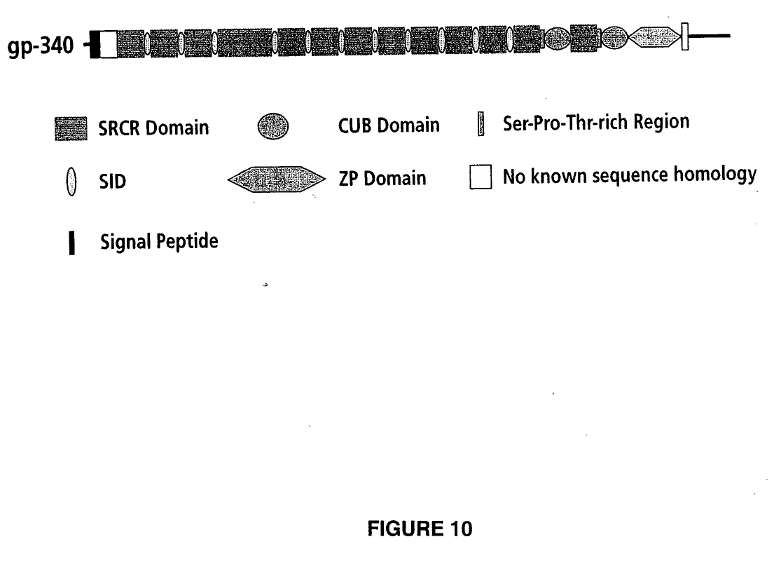

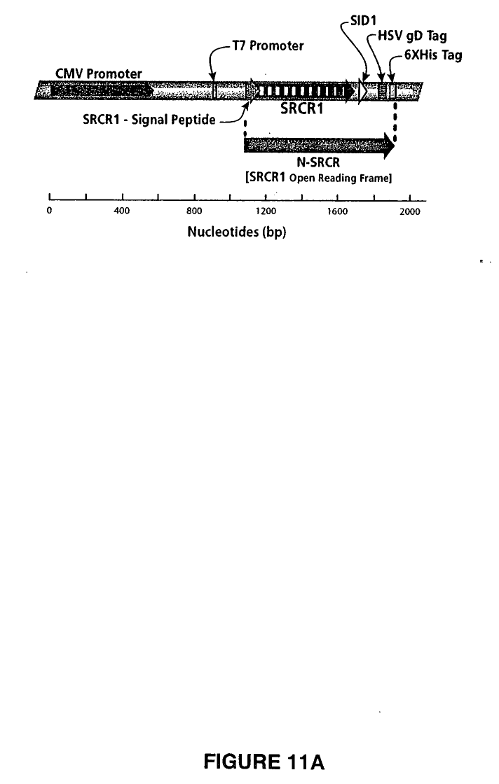

Furthermore, we reported that the major binding site for gp340 on Env involved a highly conserved sequence in the stem of the V3 loop (Wu Z W, Golub E, Abrams W R and Malamud D: gp340 (SAG) binds to the V3 sequence of gp120 important for chemokine receptor interaction. AIDS Res and Human Retroviruses 2004; 20:600-607). Since gp340 demonstrated a broad range of activity against all HIV-1 laboratory and clinical strains tested, we focused our studies on determining its active HIV binding region, with the goal of developing analogs of this structure as a microbicide. gp340 is a member of the SRCR superfamily of proteins and contains 13 highly conserved SRCR domains, each with a −110 highly conserved amino acid sequence separated by small SRCR interspersed domains (SIDs), a CUB domain, a ZP domain and one, less conserved SRCR domain (FIG. 10).

### Example 6

### Cloning of the SRCR Domain of gp340 and Inhibitory Activity against M and T Tropic HIV-1 Isolates

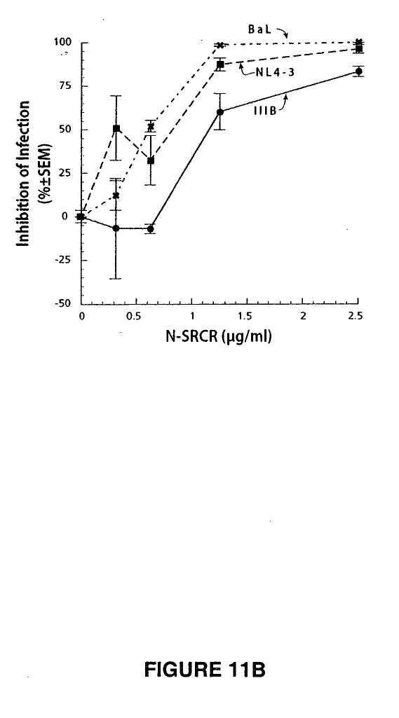

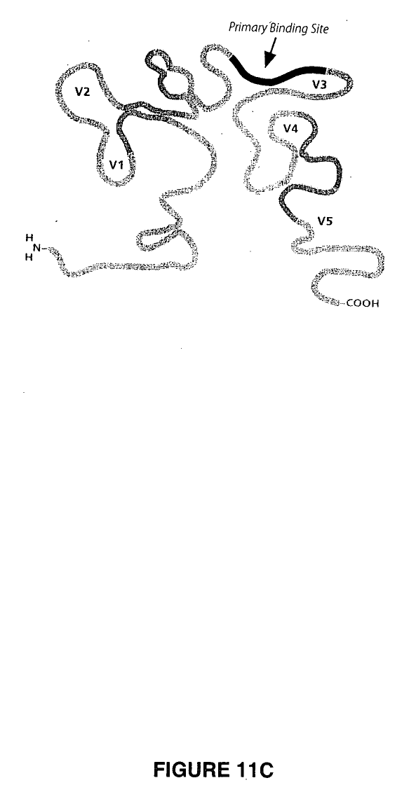

We cloned the first SRCR domain along with half of the first SID (FIG. 11A), and demonstrated inhibitory activity against M- and T-tropic HIV-1 strains, similar to the intact gp340 (FIG. 11B). To identify the binding sites on gp120 of the N-SRCR, overlapping 15 mer peptides spanning the entire length of gp120 were obtained from the AIDS Research and Reference Reagent Program. These peptides were immobilized onto a 96 well plate, and binding of intact gp340 or smaller derived molecules was determined. The 34 kDa truncated protein bound to the same regions of gp120 previously identified as the target for intact gp340 (Wu Z W, Golub E, Abrams W R and Malamud D: gp340 (SAG) binds to the V3 sequence of gp120 important for chemokine receptor interaction. AIDS Res and Human Retroviruses 2004; 20:600-607) and was also effective in blocking HIV infection with all viral strains tested (FIG. 11B). Most monoclonal and polyclonal antibodies raised to intact gp340 also recognize the N-SRCR construct. Preliminary binding data from studies of overlapping 15 mer peptides suggests that the sequence at the stem of the V3 loop (CTRPNYNKRKRIHIG (SEQ ID NO: 6) or VQINCTRPNYNKRKR (SEQ ID NO: 8), indicated in red) is the primary binding site while the yellow regions in FIG. 11C bind less gp340, and are proximal in the tertiary structure of gp120 appearing to play a secondary role in binding. To date these studies have only been carried out using an ELISA format, and in the present proposal will be confirmed and extended with biosensor studies to determine binding kinetics.

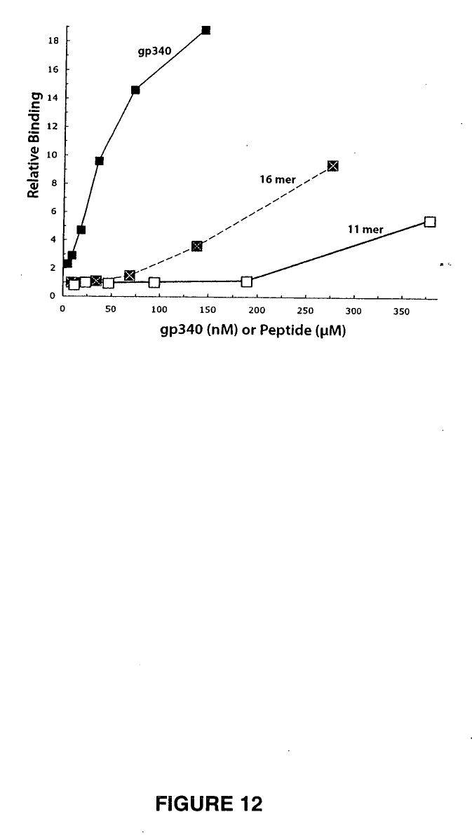

Bikker et al. studying gp340 as an agglutinin of oral Streptococci selected a consensus sequence within the 13 SRCR domains and synthesized a series of overlapping peptides spanning the entire 110 amino acid sequence of a single SRCR (Bikker F J, Ligtenberg A J M, End C, et al.: Bacteria binding by DMBTIISAGlgp-340 is confined to the VEVLXXXXW motif in its scavenger receptor cysteinerich domains. J Biol. Chem. 2004; 279:47699-47703). In their studies they identified a 16 mer (QGRVEVLYRGSWGEVC (SEQ ID NO: 7)) and an, internal 11 mer (GRVEYLXBGSW (SEQ ID NO: 2) that bound to Streptococci and promoted bacterial aggregation (Bikker, F. J., Ligtenberg, A. J., Nazmi, K., Veerman, E. C., van't Hof, W., Bolscher, J. G., Poustka, A, Nieuw Amerongen, A V. and Mollenhauer, J. (2002). Identification of the bacteria-binding peptide domain on salivary agglutinin (gp-340/DMBT1), a member of the scavenger receptor cysteine-rich superfamily. J Bioi Chem 277(35): 32109-32115.). Furthermore, by alanine scanning they subsequently demonstrated that mutations of any of only 5 amino acid (VEVL •••• W) abrogated bacterial binding activity. When we tested the 16 mer we were able to confirm the Dutch findings with binding to Streptococci and surprisingly, upon testing the binding to 15 mer peptides representing the entire sequence of gp120 we demonstrated that the gp340-derived 16 mer identified with streptococcal aggregation bound to the same peptides in Env as previously determined for the N-SRCR and the intact gp340 (FIG. 11C) with the major binding site the stem of the V3 loop (red). Note however (FIG. 12) that the binding activity of the peptides is considerably less than for the intact gp340. The present invention addresses the anti-viral activity of these peptides.

### Example 7

### Use of the SRCR Domain of M2BP for Identifying the gp120 Binding Domain

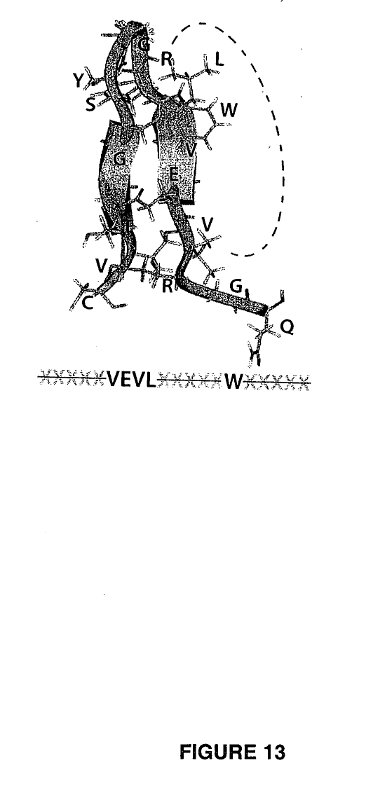

The tertiary structure of the 16 mer, which bound gp120, was modeled using the crystal structure for a comparable SRCR domain in M2BP (cyclophilin C-associated protein). Energy minimization resulted in the predicted structure shown in FIG. 13. The 5 amino acids demonstrated by alanine scanning to be essential for bacterial binding (VEVL . . . W) are all predicted to be on the same molecular face. In addition, the presence of a single aromatic tryptophan residue is useful for peptide-peptide interaction studies (see below).

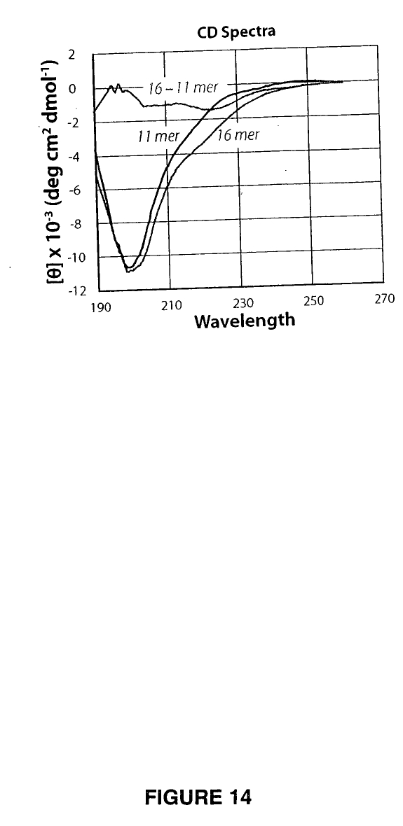

The circular dichroism (CD) spectrum of the unconstrained 16 mer, and the internal 11 mer was analyzed to determine if it was consistent with the structure predicted in FIG. 13. As shown in FIG. 14, the far UV CD of the peptides suggests that both peptides are flexible, consistent with a disordered PII chain structure with weak α or β structure superimposed. Subtracting the two spectra suggests that the two structures differ mostly in α helix content.

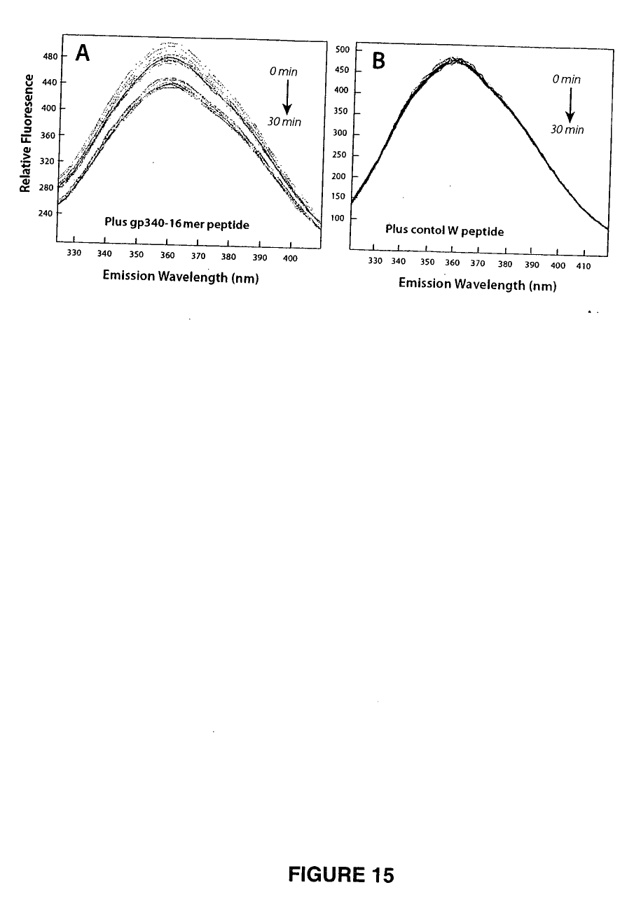

Of particular interest for the present studies is the observation that when a target peptide on gp120 (VQ I NCTRPNYNKRKR (SEQ ID NO: 8)) is mixed in solution with the 16 mer from gp340 (QGRVEVLYRGSWGEVC (SEQ ID NO: 7)), there is fluorescent quenching over time, suggesting that the trp residue becomes buried within the gp120 target site (FIG. 15A). An unrelated 15 mer with a single trp residue does not display this type of quenching, (FIG. 15B).

Although the peptide bound to the “correct” site on gp120 only modest inhibition of HIV infectivity at peptide concentrations up to 10 ug/ml were shown. While there are several possible explanations for this low bioactivity (e.g., multiple active moieties on gp340, a glycopeptide is the active structure), we hypothesized that the isolated peptide does not form or does not maintain the stable conformation predicted from the crystal structure of SRCR (FIG. 13).

### Example 8

### Methods of Stabilizing the Anti-HIV Peptides

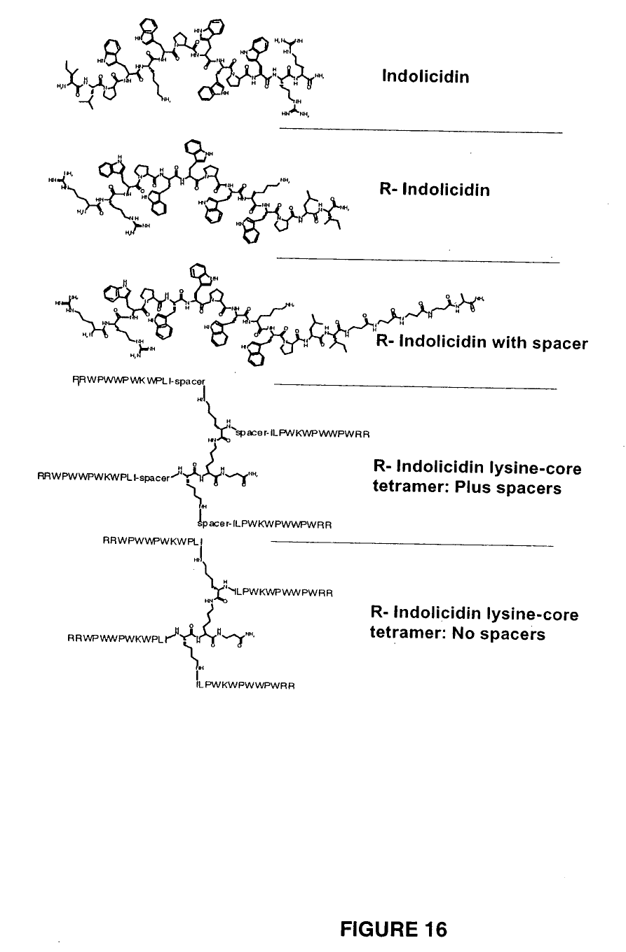

We demonstrated that covalent linkage of monomeric antimicrobial peptides based on Trp and Arg sequences offers a route to enhance the activity of peptides against several bacterial pathogens without altering cytotoxicity. The scaffolds used include dendrimeric structures as well as linear complexes comprising up to 50 monomers (FIG. 16). Peptides linked this way include the 13′ mer indolicidin which is a potent antimicrobial peptide isolated from bovine neutrophils (Staubitz, P., Peschel, A, Nieuwenhuizen, W. F., Otto, M., Gotz, F., Jung, G. and Jack, R W. (2001). Structure-function relationships in the tryptophan-rich, antimicrobial peptide indolicidin. J Pept Sci 7(10): 552-564), similar in size to the SRCR-derived peptides proposed herein. By truncating the indolicidin sequence to tetramers and increasing the number of monomers in the complexes, a 10 fold decrease in MIC values was achieved. This strategy of multivalent presentation of weak affinity ligands offers a route to overcome one potential source of the weak binding by peptides: gp340 has 13 tandem repeats of the SRCR domains. One aspect of the invention provides for enhancing the activity of SRCR-derived peptides by covalently attaching a sufficient number to an artificial scaffold.

Our working hypothesis is that the 11 and 16 mer sequences are responsible for binding to gp120, and when presented in an appropriate conformation and/or multiplicity, will inhibit HIV infectivity. Synthetic strategies, singly or in combination, are proposed to generate a set of stable and highly active and economical analogs. Synthesis and evaluation of three kinds of products will be conducted.

### Example 9

### Synthesis and Screening of Peptide Mimics

We will synthesize and screen mimetics based on active HIV-binding peptides within gp340 to create conformationally restricted small molecule inhibitors of HIV infection. These structures will initially comprise linear and disulfide linked 7-mers in order to explore the sequence space of short peptides containing permutations of the key side chains (VEVL . . . W) with and without conformational restrictions. Longer olefinic H-bond surrogates will be synthesized using the most active sequence combinations identified by a phage display screen of gp120 affinity of libraries enriched for these side chains. A second-round of phage display screening will be used to optimize this choice of final product sequences. All of the synthesized structures will be evaluated for anti-HIV efficacy and cytotoxicity. If bioactivity is low, we will increase the size of the starting peptide from a 7-mer to a 16-mer.

### Example 10

### Sequence Analysis of the HIV Binding Interface

The five side chains, VEVL ·••• W, identified in the alanine scan identified as required for binding can be presented in a large number of permutations, each presumably corresponding to a distinct set of structures. To define the sequence requirements for interaction with HIV-1, we propose to use the Ph.D.C.-7-C phage display peptide library that offers nearly complete sampling of the 20⁷=1.3×10⁹ possible 7 mer sequences. The randomized segment of the Ph.D.C.-7-C library is flanked by a pair of cysteine residues, which are oxidized during phage assembly to a disulfide linkage, resulting in the displayed peptides being presented to the target as loops. As a first step towards development of small molecules that mimic the HIV binding region of gp340, we will seek to identify small peptidic macrocycles that inhibit the target proteinprotein interaction with high affinity and selectivity. We postulate that peptide macrocycles that mimic the VEVL ·••• W motif will be found in this library. We will initially utilize the 7-mer library although the β sheet structure contains more residues. Libraries that contain longer peptides flanked by cysteines are not currently available and because identification of shorter mimetics would greatly simplify development of small molecule ligands for H IV. However, a 12 mer phage display peptide library is also available from the same company, and will be used if the Ph.D.-C-7-C does not yield sufficiently active molecules.

As supplied, the Ph.D.-C-7-C library contains 2×10⁹ independent clones, fused at the N terminus of the minor coat protein pili with a short spacer sequence GGGS. The protocol we will adopt (FIG. 10) is to add the library to gp120 or the previously identified sequence on the stem of the V3 loop immobilized on 384 well flat bottom microtiter plates (NUNC #242757). Use of the V3-peptide has the potential to increase the specificity of binding and decrease the cost of the assays. Following binding, unbound phage are removed by washing with buffer. This process is referred to as panning. These phage are amplified by growth on host cells (E. coli ER2738), and after harvesting, subjected again to the same binding assay. This methodology is widely used, although there are a number of pitfalls that could interfere including stringency of binding, dilution of the phages, and non-specific binding to the wells. However, most of these problems are averted by a preliminary exposure to untreated plastic wells before panning to eliminate nonspecific hydrophobic interactions that bind plastic alone.

After 3-4 rounds of selection, corresponding to 1:10³-⁴ per round, individual clones are selected and their sequences determined using the primer provided to define the sequences that have emerged. Sequencing up to 25 of the selected phage can then reveal essential information concerning the sequence requirements for unconstrained peptide binding to gp120. Several outcomes are possible: (i) the sequences show no conserved side chains at all. (ii) the sequences show conserved side chains but not VEVL •••• W. (iii) the sequences show conservation and include VEVL •••· W. Through this experiment, we will discern whether or not alternative sequences to VEVL •••· W have promise for further microbicide studies, and confirm or reject the hypothesis that VEVL •••· W in a conformationally restricted background offers an optimal lead for chemical synthetic mimetics.

As a second stage experiment, we will use a 12 mer untethered PhD.-12 library that offers a selection of possible 12 mer sequences to complement the 7 mer restricted library described above. This library of longer sequences is not comprehensive, but overcomes the problem of size. The procedures to be used for the second screening are essentially identical to those described for heptamers: repeated cycles of binding, panning, enrichment and finally sequencing samples of the third to fourth round phages to identify common patterns.

### Example 11

### Preparation of Conformationally Restricted Analogs

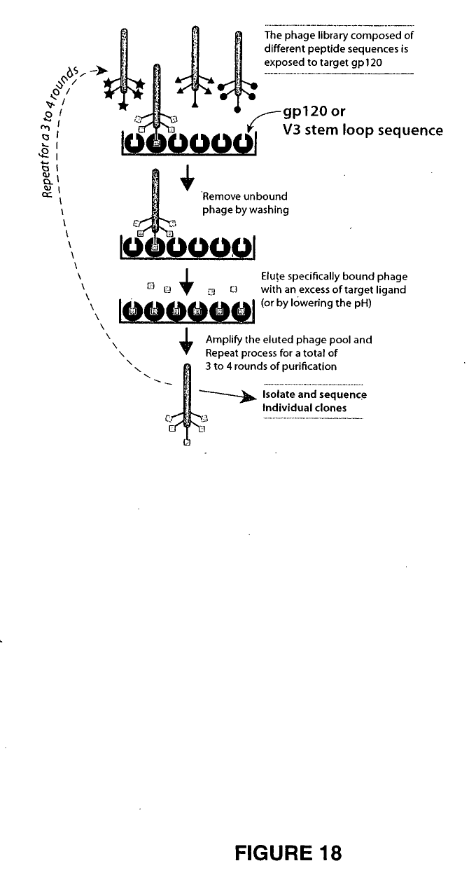

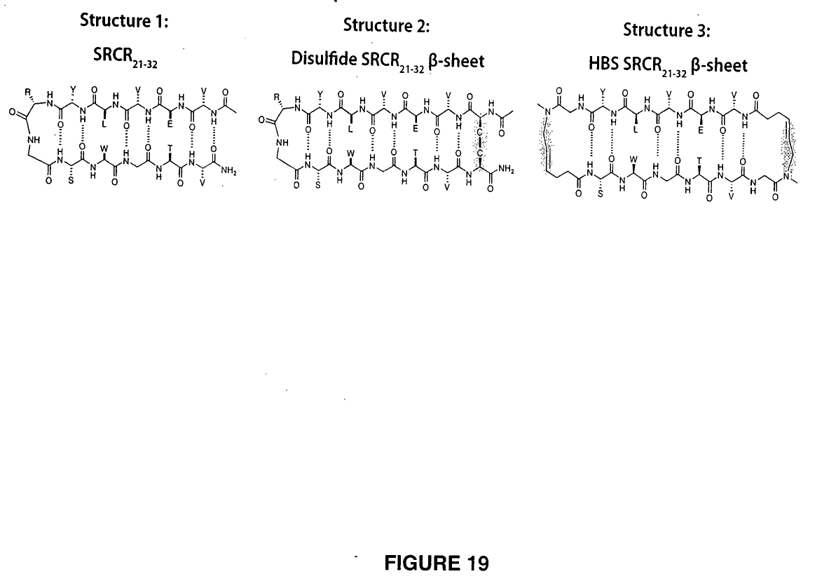

The scheme below (FIG. 19) illustrates 3 alternative constructs that we propose to synthesize and evaluate for anti-HIV activity. The compounds shown in FIG. 18 incorporate the parental SRCR21-32 and two beta sheet mimics of that peptide, one in which disulfides are introduced to stabilize the structure and the second which utilizes olefinic H-bonds. It should be noted that any tighter binding sequences that emerge from the phage display experiments will be substituted for those shown.

Peptides will be synthesized by standard Fmoc solid-phase synthetic chemistry, purified by reversed phase HPLC and characterized by mass spectrometry. The solution conformation of each compound will be determined by circular dichroism and 2D NMR spectroscopy: beta strands have a characteristic minimum in the CD at 218 nm and show strong NOE's between adjacent Cα and NH protons. The disulfide hairpin 2 will be synthesized from the linear peptide following standard methods for the formation of the disulfide link (Annis, I., Hargittai, B. and Barany, G. (1997). Disulfide bond formation in peptides. Methods Enzymol 289: 198-221). The β-Sheet mimetic 3 contains two covalent bonds in place of the inter-strand hydrogen bonds. Replacement of the interstrand hydrogen-bonds with covalent links is expected to provide a highly stable β-sheet mimetic. This method differs from the disulfide approach used in hairpin 2 and other commonly employed side-chain cross-linking strategies because all side-chain functionality required for molecular recognition remains intact while no external appendages that may block recognition are placed on the molecule. Modeling studies suggest that the constraint in the second peptide will stabilize the sheet significantly without introducing gratuitous side chains that might reduce the affinity of the peptide for target. In each case the five essential side chains (VEVL •••· W) identified in the alanine scan of SRCR are conserved on one face.

### Example 12

### Synthetic Path for Construction of HBS SRCR B-Sheet

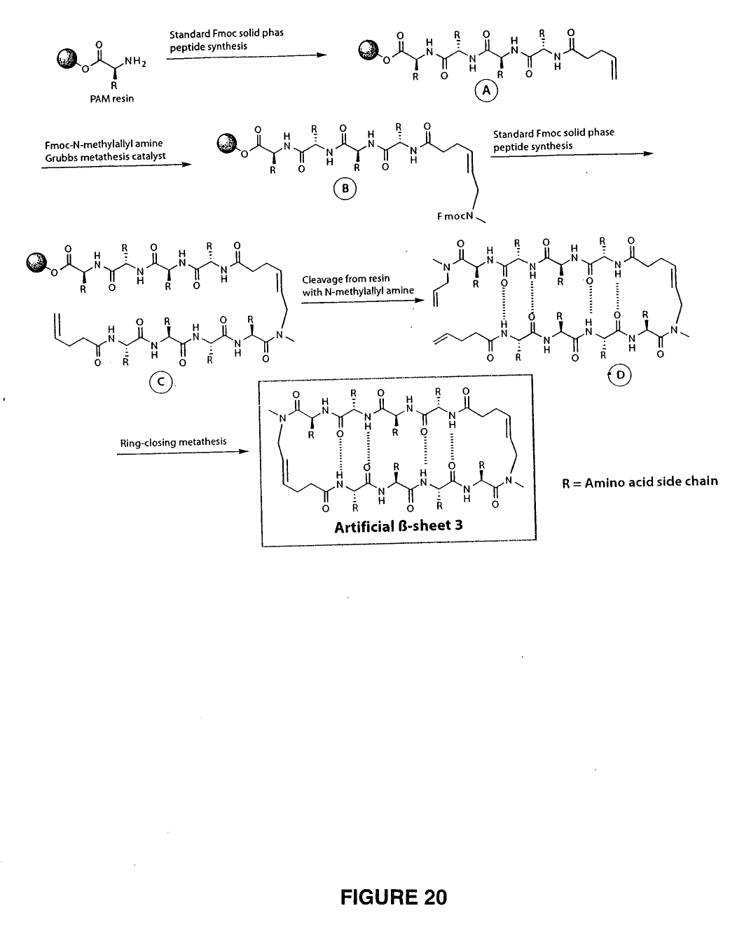

The hydrogen-bond replacement approach for the synthesis of stable α-helices that we will use has been utilized successfully in the past (Chapman, R. N., Dimartino, G. and Arora, P. S. (2004). A highly stable short alpha-helix constrained by a main-chain hydrogen-bond surrogate. J Am Chem Soc 126(39): 12252-12253; Dimartino, G., Wang, D., Chapman, R. N. and Arora, P. S. (2005). Solid-phase synthesis of hydrogen-bond surrogate-derived alpha-helices. Org Lett 7(12): 2389-2392; Wang, D., Liao, W. and Arora, P. S. (2005). Enhanced metaolic stability and protein binding properties of the hydrogen-bond surrogate-derived artificial alpha-helices: Application to Bcl-xl. Angew Chem Int Ed 44: In press). Structure 3 will be synthesized in an analogous manner utilizing the ring-closing metathesis reaction as described (Dimartino, G., Wang, D., Chapman, R. N. and Arora, P. S. (2005). Solid-phase synthesis of hydrogen-bond surrogate-derived alpha-helices. Org Lett 7(12): 2389-2392). The synthetic scheme is diagramed in FIG. 20. Briefly, tetrapeptide A capped with pentenoic acid will be synthesized by standard peptide synthesis methods on PAM resin. Olefin metathesis of A with Fmoc-N-methylallyl amine will afford compound B which will be elongated and capped with another pentenoic acid monomer to obtain C. Cleavage of C with N-methylallyl amine followed by a ring-closing metathesis step is expected to provide the artificial B-sheet 3. It will be purified by reversed-phase HPLC and characterized by LCMS and circular dichroism and 20 NMR spectroscopies (including TOCSY and NOESY). Methods for synthesis, HPLC purification and proof of structure using MS and NMR have all been published (Chapman, R. N., Dimartino, G. and Arora, P. S. (2004). A highly stable short alpha-helix constrained by a main-chain hydrogen-bond surrogate. J Am Chem Soc 126(39): 12252-12253; Dimartino, G., Wang, D., Chapman, R. N. and Arora, P. S. (2005). Solid-phase synthesis of hydrogen-bond surrogate-derived alpha-helices. Org Lett 7(12): 2389-2392; Wang, D., Liao, W. and Arora, P. S. (2005). Enhanced metabolic stability and protein binding properties of the hydrogen-bond surrogate-derived artificial alpha-helices: Application to Bcl-xl. Angew Chem Int Ed 44: In press).

### Example 13

### Synthesis of Multivalent Molecules from Reactive Peptide Polymers

We will create polyvalent structures from SRCR-derived peptides identified above, the goal being to enhance avidity by coupling peptides and/or mimetics to dendrimeric and linear scaffolds (Mammen, M., Choi, S. K. and Whitesides, G. M. (1998). Polyvalent interactions in biological systems: Implications for design and use of multivalent ligands and inhibitors. Angewandte Chemie-International Edition 37(20): 2755-2794) Just as chelation dramatically enhances metal binding of EDTA by increasing the avidity of weakly interacting acidic ligands, a similar strategy can promote tighter binding of complexes to biological targets such as cell surfaces (Kiessling, L. L., Gestwicki, J. E. and Strong, L. E. (2000). Synthetic multivalent ligands in the exploration of cell-surface interactions. Curr Opin Chem Biol 4(6): 696-703). An alternative strategy based on the 13 SRCR repeats in gp340, is to generate multiple binding mimetics on a single polymeric construct.

Antiviral linear peptide polymers are based on the reactive linear scaffold PMA-polymaleic anhydride covalently linked to antiviral peptide monomeric sequences (Gaylord, N. G. (1975). Poly(Maleic Anhydride). Journal Of Macromolecular Science-Reviews In Macromolecular Chemistry And Physics C 13(2): 235-261). The peptide monomers will be synthesized using standard Fmoc solid phase chemistry and the N termini capped for comparison with the polymeric species. PMA is supplied at an average molecular weight of 5000 (Polysciences, Inc., PA), corresponding to 50 maleic anhydride units per chain. The multivalent peptide complexes will be derived by the conjugation of the maleic anhydride moieties with free N termini (Tsiourvas, D., Paleos, C. M. and Dais, P. (1989). Functionalized Polymers Derived From The Reaction Of Polymaleic Anhydride With Amines And Alcohols. Journal Of Applied Polymer Science 38(2): 257-264) of monomeric peptides whose side chains are protected to avoid cross-linking. Free carboxylate groups will be methylated. See schemes 1, 1A, 2 and 2A. The final polymer product has a series of branches that are either peptides or acrylate, which is confirmed by 1H NMR. As a control for the polymer backbone itself, a sample of the polymer is fully substituted with the addition of methylamine instead of peptides. The extent of substitution in each polymer will be calculated by integration of 1H NMR peaks. The NMR resonances used for composition determination include the reference peak at 63.4-3.8 (assigned to CO₂Me of the backbone). The integrals of peptide peaks relative to the backbone will be used to determine the residue ratio. The molecular weight distribution in each polypeptide will also be determined using a gel permeation chromatography column. The products will be purified by HPLC, and the composition verified by MS and NMR analysis of the ratio of polymer to resonances in the monomer (e.g., trp).

The advantage of these polyvalent constructs is the ease of large scale synthesis, since the PMA is available commercially and synthesis of monomer peptides is inexpensive ($12/gm for short chain peptides). For the antiviral constructs we will explore further truncations of SRCR peptide analogs to further reduce costs. We also plan to explore alternative polymeric backbones and variation in the ratio of peptide to unfilled polymer sites to optimize bioactivity.

Monomeric peptides are assembled on Sieber Amide resin by using standard Fmoc solid phase synthesis without capping N terminus at the last step of synthesis. After completion of synthesis, peptides are cleaved from resin with 1% TFA in dichloromethylene. The conjugation reaction between PMA (50 maleic anhydride units/polymer chain) and excess peptides is carried out overnight at 40° C. with agitation on a rotary shaker in DMF catalyzed by DIEA. The unreacted maleic anhydride groups are quenched by the addition distilled and deionized water, the product treated with excess trimethylsilyldiazomethane (TMDM) to methylate the resulting carboxylic acid groups, dialyzed overnight (3500 molecular weight cut off, 1000 mL) in distilled water and polymeric peptides isolated. Control polymers are generated by the addition of methylamine instead of peptides and isolated by the steps described above. The cleavage of side chain-protecting groups from the polymer is performed with 95% TFA, 2.5% TIS and 2.5% H₂O. The final product is precipitated with cold ether, lyophilized to dryness, and its composition confirmed by ¹H NMR.

### Example 14

### Synthesis of Dendrimeric Constructs

An alternative to synthesis of linear polyvalent constructs involves the use of more highly branched dendrimers. Based on our experience with indolicidin dendrimers, we will design and prepare dendrimers using SRCR-derived bioactive peptides.

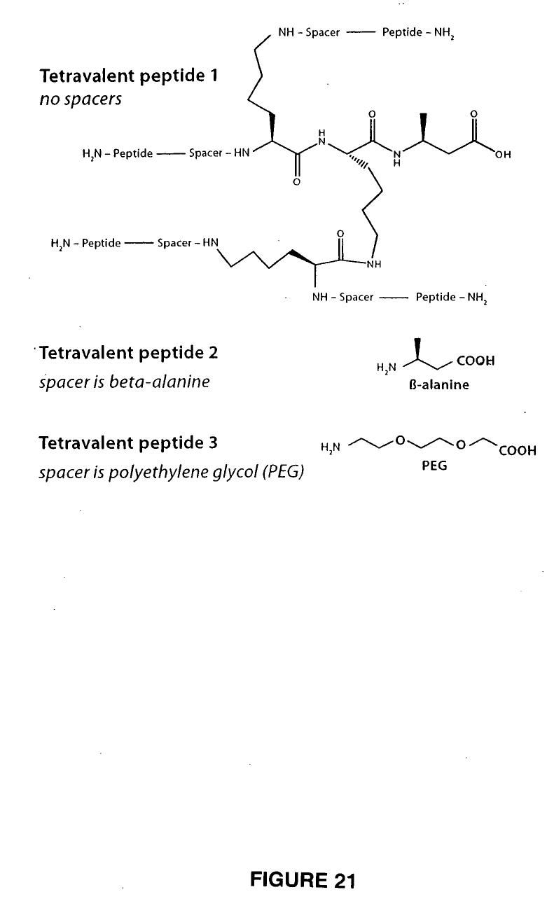

The synthesis of the tetravalent peptide structures 1, 2, 3 (FIG. 21) utilizes a template composed of three residues of lysine. Synthesis is initiated by coupling an Fmoc-protected ˜-alanine residue, making up the C-terminus, to Wang resin. This residue is deprotected and coupled to Fmoc-Lys (Fmoc)-OH with Fmoc-protected a- and E-amino groups. Using the same protection group for both amino groups allows for simultaneous deprotection and formation of two amide bonds during the next coupling step, which results in a branched peptide. A tetravalent core is achieved by repeating the coupling to Fmoc-Lys (Fmoc)-OH with Fmoc-protected a- and E-amino groups, so that both amino groups of the first lysine are coupled to lysine residues and once again protected with two Fmoc groups. After deprotection, Fmoc-protected PEG or ˜-alanine spaces is coupled to the four NH₂ groups of the tri-lysine template, followed by additional amino acids using an automated peptide synthesizer. Crude peptide solutions are routinely deprotected with 94% TFA (trifluoroacetic acid), 2.5% H₂O, 2.5% TIS (triisopropylsilane), 1% EDT (ethanedithiol), precipitated in ether and HPLC purified on a C18, 300 A reverse phase column.

Dendrimeric Tetrapeptides

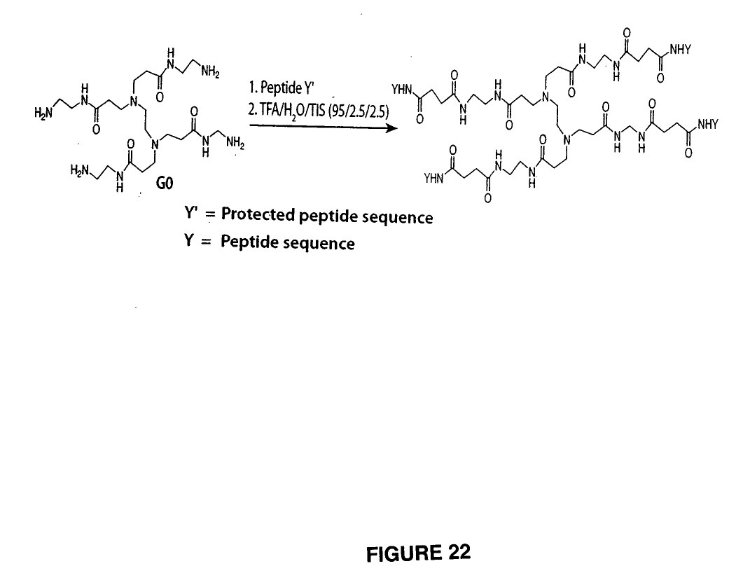

The synthesis of polyamidoamine (PAMAM) dendrimer-peptide conjugates PAMAM-Y, where Y is the peptide, GO, G1 and G2 generations are achieved by first synthesizing monomers with succinic anhydride capping at the N-termini (FIG. 22). The subsequent addition of the monomers onto PAMAM's reactive surface sites is effected by reacting the PAMAM's amine groups with carboxylic acid groups from monomers capped at the N terminus with succinic anhydride forming amide bonds in the presence of coupling reagents DIEA (diisopropylethylamine). The reaction mixture is shaken at 3TC for 48 hrs, dialyzed (3500 MW cutoff, 1000 mL) against distilled water, and then lyophilized to dryness. Side-chains are deprotected with 95% TFA, 2.5% H₂O, and 2.5% TIS. Molecular weights are verified by time of flight mass spectrometry (Bruker Maldi-TOF spectrometer).

### Example 15

### Quantitative Assessment of the SRCR Analogs and Multivalent Complexes

Each of the initial structures produced by the aforementioned methods will be tested in vitro for biologic activities. These assays fall into two general types: inhibition of HIV infectivity and binding to gp120. In addition, cytotoxicity studies using both the HIV target cells and ME-180 cells will be carried out. All studies will be performed over a wide concentration range (nM-μM) and data expressed as concentration of structure giving 50% binding or 50% inhibition as compared to the N-SRCR.

A. Measurement of Anti-HIV-1 Activity

i) HIV-1 Inhibitory Assays for Cell-Free Virus

Various assays will be employed for testing for inhibition of HIV infectivity, e.g. GFP-based assays using either reporter viruses with fluorescent protein genes that produce fluorescence upon infection of cells, or Jurkat cells transfected with an LTR-EGFP (Enhanced GFP) reporter construct (referred to as JLTRG-R5 cells) that express minimal EGFP when uninfected, but respond to HIV-1 infection with extremely high levels of EGFP fluorescence (Kutsch, O., Levy, D. N., Bates, P. J., Decker, J., Kosloff, B. R., Shaw, G. M., Priebe, W. and Benveniste, E. N. (2004). Bis-anthracycline antibiotics inhibit human immunodeficiency virus type I transcription. Antimicrob Agents Chemother 48(5): 1652-1663; Ochsenbauer-Jambor, C., Jones, J., Heil, M., Zammit, K. P. and Kutsch, O. (2006). T-cell line for HIV drug screening using EGFP as a quantitative marker of HIV-1 replication. BioTechniques 40(1): 91-99). Since the cells express both CXCR4 and CCR5 co-receptors they can be used with a wide range of M-tropic and T-tropic viruses. In addition, the wide dynamic signal range and low background facilitates micro well plate-based assays. Also, a series of HIV reporter viruses that direct expression of GFP or other fluorescent proteins (CFP, YFP) during productive infection will be employed for these studies. These reporter viruses allow measurement of infection as a % of cells that become fluorescent, (Le., inhibited), in the presence of drug and will be a valuable tool for subsequent studies with vaginal explants or mixed blood cell cultures. Both of these fluorescence based technologies are ideal for high-though-put assays and have been used in FACS analyses as well as 384-well plate assays monitored with a fluorometric plate reader (Bio-Tek Synergy HT). Our plan is to use the JITRG-R5 assay for preliminary screening of drugs and dose ranging studies and then confirm these findings with a more traditional assay, described below.

Plate based assays are carried out in 384-well optical bottom black plates with a final concentration of 1×10⁵ cells/well in a final volume of 90 μl of phenol red-free RPMI1640 (supplemented with glutamine, penstrep, and 2% heat-inactivated FCS). Test inhibitor (10 μl) is added to 40 μl of cells and then 50 μl of virus adjusted to an MOI of 0.1 is added. Negative controls lack drug, positive controls contain virus plus AZT, and test drugs are initially evaluated at 10 fold dilutions over 3 log concentrations. When active inhibitors are identified, these studies will be repeated with drug concentrations at 2-fold dilutions over the effective dose range. Data will be analyzed to generate an effective dose (ED), ED₅₀ and ED₉₀ for each drug (nM-μM). Plates are analyzed for EGFP expression at 24 hr intervals for 5 days. For initial studies HIV-1 strains IIIB and Bal will be used.

To confirm the data obtained with the EGFP cells we will employ the assay currently utilized in our laboratory. Virus is treated for 30 min with purified gp340, or each of the small molecule inhibitors in serial dilutions of RPMI-1640 containing 10% FCS, 1 mM CaCl₂ and 2 μg/ml polybrene. Negative controls contain virus only, positive controls contain virus plus AZT (10 nM). Either I1-2 stimulated primary human PBMCs or H9 cells are added and the infection carried out for 18 hrs before residual virus and inhibitors are removed by washing with culture medium. Cells are re-fed with the same concentration of inhibitor, and p24 levels determined after 5-6 days of culture. Results are expressed as % inhibition relative to the negative controls allowing the calculation of an effective dose (ED₅₀) for each active. All experiments will be carried out with HIV-1 strains IIIB, Bal, and N14-3. Positive findings will be confirmed by additional tests using primary clinical isolates, preferably derived from the lower reproductive tract.

B. Measurement of Binding Activity to Target Peptides from gp120

i) gp120 Binding Assays

96-well U-bottom vinyl plates are coated with gp120, or gp120-derived peptides derived from the stem of the V3 loop (2.5 μg/50 μl, 100 mM Na bicarbonate buffer, pH 9.6 at 4° C., overnight). Plates are washed 4× and blocked with 2% blotting grade non-fat milk (BioRad). For negative controls, a fixed amount of intact gp340 is added to the wells, the amount selected to give an ΔA of 1.5.

In the remaining wells, N-SRCR, 16 mer-derived peptide, or each of the analogs synthesized in this proposal are added to the plates for 60 min, followed by addition of intact gp340 and binding detected using antibody to gp340. Active molecules will compete with gp340 binding to gp120 sites and thus decrease the observed absorbance, relative to the controls using gp340 only. The binding activity of analogs will be expressed as a percent of competition produced by the original 16 mer. We anticipate that many of the stabilized or polymeric analogs will prove to be stronger competitors than the original 16 mer derived from the SRCR.

ii) Kinetics of Binding of Small Molecule Inhibitors to gp120.

Because the plate assays described above only give approximate binding activity data we will also carry out a series of biosensor binding studies. Earlier studies demonstrated that gp340 bound to sites on gp120 distinct from sCD4, such that binding of the two proteins was Ca++ dependent, and identified monoclonal antibodies to gp340 that inhibited the binding of gp340 to gp120 (Wu et al., 2003).

In the present study we will take the “best” SRCR-derived small molecule inhibitors and evaluate their real-time kinetics using BiaCore (surface plasmon resonance detection) technology. Two types of studies will be carried out: (1) direct binding studies with gp120 and the analogs, and (2) competition studies to determine the ability of the analogs to block binding of the intact gp340. For these experiments we start by capturing gp120 on the sensor chip and studying the binding of the small molecule inhibitors, and then compare this to the reverse situation, where the drugs are immobilized and we monitor gp120 binding. The latter approach has the advantage of using less gp120, and also of giving a larger signal due to the higher molecular weight of the viral glycoprotein. In earlier studies evaluating binding of gp340 to gp120 we determined a Kd of 5.8 nM, comparable to the affinity for sCD4. We predict that the small molecule inhibitors will have similar affinities. Depending on the experimental outcome, we may make additional modifications in the synthesis of mimetics to increase the affinity for gp120 targets. Once optimal conditions for binding and HIV inhibition have been determined, the experiments will be extended to compare the active analogs to a series of anti-HIV 1 microbicide candidates (e.g., T-20, CV-N, 12p1, UC781) to assess their potency as compared to other microbicidal candidates

C. Potential In Vitro Cytotoxicity

i) HIV-1 Inhibitory Assay for Cell-Associated Virus

Inactivation of cell-associated HIV will be tested using Sup T1 cells infected with HIV-1 IIIB. In these assays, cells are infected with virus and 72 hrs later, cells are centrifuged, washed and resupended in RPMI1640 at a concentration of 1×10⁶ cells per 90 μl. Drugs to be tested are diluted in medium to the desired concentration and added in 10 μl aliquots to infected Sup T1 cells for 60 min, then diluted and added to P4-CCR5 MAGI cells for an additional 48-72 hrs. At this time, lysates are assayed for B-galactosidase activity. Alternatively, Molt-4 cells chronically infected with BaL will be used. Drugs are added to these cells and washed, as above. Then −5,000 Molt-4 cells are mixed with 250,000 JLTRG-R5 cells in 384 well plates, and GFP is measured 4 days later to assess the effect of the drugs on cell associated virus. This latter assay has been optimized and used successfully by the UAB group.

ii) Cytotoxicity Assays

To assess potential in vitro toxicity of each of the agents, host cells (PBMCs or H9 cells) and cervical epithelial cells (ME-180) are incubated with serial dilutions of each molecule for 4 and 24 hrs. Cells are then washed and assessed for viability with the CellTiter96® AQᵤₑₒᵤₛ cell proliferation assay (Promega). In this assay, the tetrazolium compound (3-4,5-dimethylthiazol-2yl)-2,5-diphenyltetrazolium bromide) is reduced by cells into a soluble formazan product and its absorbance monitored (AA 490 nm). Results are used to determine the toxicity, if any, of each of the molecules. Data are expressed as the LD₅₀. These values are then used to calculate the therapeutic index (LD₅₀/ED₅₀), a relative value that is useful in comparing the set of gp340-derived small molecule HIV inhibitors. The MTT assay is used for distinguishing live from dead cells. Any synthesized agents that do not kill mammalian cells will be further evaluated using the more sensitive cytokine assays (Fichorova, R. N., Bajpai, M., Chandra, N., Hsiu, J. G., Spangler, M., Ratnam, V. and Doncel, G. F. (2004). Interleukin (IL)-1, IL-6, and IL-8 predict mucosal toxicity of vaginal microbicidal contraceptives. Biol Reprod 71(3): 761-769). These ELISA assays, utilizing commercially available kits (R&D systems, Minneapolis Minn.), measure the cytokine levels (typically IL-1, IL-6, and IL-8) in culture supernates. Cells will be exposed to each of the agents at a range of dosages above and below the ED₅₀ for 24 hours and the supernatants will be analyzed in comparison to control wells to assess the effects on cytokine released. Data will be expressed as pg cytokine/ml culture supernatant or per 10⁶ viable cells (as determined by the MTT assay).

### Example 16

### Evaluation Matrix

We will generate an evaluation matrix to assist in the process of selecting the gp340 mimic to develop into microbicides. This matrix will include efficacy data, cytotoxicity, therapeutic index, ease and cost of synthesis.

To facilitate the selection process we have designed a preliminary evaluation matrix. As drug candidates become available, they will be given a drug number, and entered into the form. We believe that drug efficacy (ED₅₀) cytotoxicity (LD₅₀) and the therapeutic index will be the 1^{st} tier parameters in making a decision as to which drugs to advance into the R33 phase. The 2^{nd} Tier parameters will be scored with a numerical scale (1-4).

### Example 17

### Release Characteristics of Candidate Microbicides, and Determination of Anti-HIV Properties, Cytotoxicity, and Effects of Exogenous Proteins on the Bioactivity of Formulated Drug

As noted herein, various formulations are contemplated for delivery of the active anti-HIV peptide, or derivative or active fragment thereof, for example, a vaginal gel. There are several options for detecting drugs released from the formulation. Isotopic labeling has the advantage of ease of detection, but there may be regulatory issues dealing with isotopic transport. In one embodiment, the ATTO-TAG system (Molecular Probes) will be used to bind to primary amines on the peptide mimetics. The synthesized peptide derivatives are incubated with KCN and a CBQCA reagent for 1 hr at room temperature at pH 8.5-9.5. The resulting products are maximally excited at 450 nm with an emission spectrum centered at ˜550 nm. The advantage of this highly fluorescent type of labeling is that it can be carried out after the dissolution of the formulation and the drugs are not modified until the time of detection. The formulation and dissolution assays described monitor both drug release and stability of the active agent after the formulation. In studies described below, we will also evaluate the formulated agents using ex vivo tissue.

To be an effective microbicide, a drug must have activity against a wide group of X4 and R5 viruses including clinical isolates and resistant viruses. Effective formulations will be tested against a panel of HIV-1 laboratory and clinical isolates, including representatives from each clade and a series of multi-drug resistant HIV-1 viruses. Most of these are available from the AIDS Research and Reference Reagent Program. Primary isolates from Group M with Env subtypes A, B, C, O, E, and F and also Groups N and O will be used. In addition activity against RT, protease, and multi-drug resistant HIV-1 will be evaluated, as will clinical isolates obtained from the lower reproductive tract. In each case the drug/formulation will be titered over a wide range of concentrations and the ED₅₀ determined as compared to neat drug. Data will be entered into an evaluation matrix in order to distinguish between formulations with broad anti-HIV activity from those with more limited activities.

For cytotoxicity evaluation the effects of neat and formulated drugs on both tissue culture cells and with vaginal/cervical tissues ex vivo will be studied. In the cell based assays the ME-180 cells will be used as the target and assay viability with the standard MTT assay as described. However, this is a live-dead assay, and agents which “pass” the MTT assay may still cause the release of inflammatory cytokines. Once again, each agent (i.e., drug, formulation, released drug) will be titered over a wide range of concentrations and data expressed as LD₅₀ for triplicate determinations.

Since many drug candidates are inactivated by serum, the effects of its addition as well as cervical vaginal lavage on the bioactivity of the SRCR analogs will be evaluated. For these studies, the GFP assay will be used for determining anti-HIV activity. In each case, virus is incubated with serum or CVL for 30 min, and the mixture then added to test cells. Serum and CVL will be collected (=100%) and serially diluted prior to testing. Each of the drugs (neat and formulated) will be incubated with serum or CVL for 30 min, and then tested for anti-HIV activity as compared to the drugs incubated with buffer only. Percent inhibition by added proteins will be calculated by the difference between drugs plus serum or CVL and drugs in buffer. Information that the activity of any of the agents being developed is compromised by serum or CVL will be added to the evaluation matrix and interpreted with the aid of QSAR modeling, and may lead to changes in the lead drug selected, or to the synthesis of modified mimetics.

### Example 18

### Antibacterial and Spermicidal Activity of Selected Microbicide Candidates

The effect of the SRCR analogs synthesized will be tested for broad anti-microbial activity including gram positive and gram negative organisms, for example, Streptococci and Helicobacter, as well as others. In addition, the analogs will be tested to determine whether the normal flora of the vagina can be maintained, for example, lactobacilli. While intact gp340 can agglutinate numerous bacterial species, it does not appear to be bacteriocidal. However, this has not been investigated in detail by us or others, and furthermore, the small molecular weight mimetics may have different bioactivities than the entire gp340. To this end, we will determine MIC (Minimum Inhibitory Concentration) values for the small molecule inhibitors using Lactobacillus as the primary target organism. For these assays we will employ a 96-well plate assay with 100 μl of medium, 100 μl of test drugs in serial dilution (in triplicate). Lactobacilli (˜1.5×10⁶ bacterial/10 μl) are then added to the wells, giving a final concentration of ˜1.5×10⁵ bacteria per well. Plates are read visually after 24-48 hrs to determine the MIC. To determine the Minimum Cytotoxic Concentration (MCC), aliquots from wells on each side of the MIC are streaked onto agar plates. The MCC is the lowest concentration that shows no bacterial growth. While we do not believe that these candidate microbicides will have anti-bacterial activity, we will also look at their effect on target STIs including Chlamydia trachomatis, Neisseria gonorrhoeae, and Treponema pallidum. If any of the analogs demonstrate significant bacteriostatic or bacteriocidal activity, we will do further analysis to understand the mechanism, identify HIV inhibitors that lack anti-bacterial activity, or devise methods to prevent depletion of endogenous Lactobacilli. We will also look at the effect of the analogs on viral STIs such as Herpes Simplex Virus (HSV) and Human Papilloma Virus (HPV). In all cases we will test the neat drug, formulated drug, and placebo for anti-bacterial activity.

Testing will also be done to determine whether the analogs have spermicidal activity. To that end, we will carry out in vitro spermicidal testing of the candidate microbicides, both neat and formulated, in collaboration with CONRAD. This organization has refined the in vitro tests to include a modified Sander-Cramer assay, a double-end test carried out in capillaries, and sperm motility and viability tests (Fichorova et ai, 2004).

### Example 19

### Drug Absorption and Toxicity Studies Using Ex Vivo Vaginal/Cervical Tissues

There are several model systems to evaluate in vitro effects on vaginal/cervical tissues including the models reported by Shattock (Watts, P., Nuttall, J., Coplan, P., Fairhurst, D., Mitchnick, M. and Shattock, R. (2004). Concordance between different in vitro culture models designed to predict vaginal irritation and or toxicity. Microbicides 2004. London, England), and Gupta (Collins, K. B., Patterson, B. K., Naus, G. J., Landers, D. V. and Gupta, P. (2000). Development of an in vitro organ culture model to study transmission of HIV-1 in the female genital tract. Nat Med 6(4): 475-479), and tissue systems available from MatTek Corporation. We propose to carry out initial testing using vaginal-ectocervical tissue (MatTek) to follow HIV infection and inhibition, uptake of drug into tissue and cytotoxicity/cytokine release.

MatTek has developed a tissue culture model using human vaginal-ectocervical (VEC) and dendritic cell co-cultures to form a 3-D tissue system (Ayehunie, S., Cannon, C., Lamore, S., Bellevance, K., Kubilus, J., LaBonte, J., Pudney, C., Anderson, D., Sodroski, J. and Klausner, M. (2003). Reconstructed human vaginal-ectocervical tissue models. Topical Microbicides Development and Evaluation Workshop, Washington, D.C., USA, NIH; Ayehunie, S., Cannon, C., Lamore, S., Bellavance, K., Kubilus, J., LaBonte, J., Owens, J., Pudney, C., Anderson, J., Sodroski, D., Klausner, M. and Sheasgreen, J. (2004). Human Vaginal-ectocervical tissue model for microbicide irritation studies. Microbicides 2004. London, England). The tissue can be mounted within a tissue culture well so drug can be added on one side of a porous membrane, and fluid samples can be collected from the other side.

Several investigators have reported using the MatTek system to study HIV infection, cytotoxicity, and cytokine release. We will add each of the test drugs, or placebo to the top of the tissue, and monitor cytotoxicity and cytokine release as described by Watts (Watts, P., Nuttall, J., Coplan, P., Fairhurst, D., Mitchnick, M. and Shattock, R. (2004). Concordance between different in vitro culture models designed to predict vaginal irritation and or toxicity. Microbicides 2004. London, England). For a positive control we will use Nononxynol-9, and for a negative control either buffer (in the case of neat drug) or placebo (with formulated drugs). Cytokines to be monitored in the medium include IL-1, IL-6, and IL-8, using commercial kits (R&D system). Tissue viability will be assessed with the MTT assay as described previously. We will also characterize the potential immunoinflammatory properties of our agents. We will use vaginal or buccal tissue in a Franz cell to monitor the interaction of neat drugs and formulated microbicides with mucosal tissue to estimate if uptake in vivo is likely. These ex vivo methods have been successfully developed to analyze drug permeation into skin, nasal and buccal mucosa (Lee, J. and Kellaway, I. W. (2000). Buccal permeation of [D-Ala(2), D-Leu(5)]enkephalin from liquid crystalline phases of glyceryl monooleate. Int J Pharm 195(1-2): 35-38; Lopes, L. B., Brophy, C. M., Furnish, E., Flynn, C. R., Sparks, O., Komalavilas, P., Joshi, L., Panitch, A. and Bentley, M. V. (2005). Comparative study of the skin penetration of protein transduction domains and a conjugated peptide. Pharm Res 22(5): 750-757; Ventura, C. A., Giannone, I., Musumeci, T., Pignatello, R., Ragni, L., Landolfi, C., Milanese, C., Paolino, D. and Puglisi, G. (2005). Physico-chemical characterization of disoxaril-dimethyl-beta-cyclodextrin inclusion complex and in vitro permeation studies. Eur J Med Chem). Briefly, tissue is equilibrated in a Franz cell by adding 0.5 ml PBS to the donor and 2.5 ml PB to the receiver compartment. The chamber is incubated at 37° C., and then drug is added to the donor compartment, and aliquots of the solution in the receiver are sampled for drug using the ATTO-TAG post-labeling method described above. This allows one to calculate the kinetics of permeation of drug through a mucosal surface. Preliminary studies will be carried out with commercially available porcine buccal tissue. In addition we will monitor the penetration of drug into the tissue using fluorescent microscopy of tissue sections. Subsequent studies will utilize vaginal/cervical tissue obtained from MatTek. The purpose of these experiments is to estimate if systemic uptake is likely in vivo. If formulated or unformulated material does indeed penetrate intact mucosae, we will assess the effects of added serum on this process.

### Example 20

### Quantitative Structure Activity Relationships (QSAR's)

In order to understand the roles of specific residues, and perhaps design more potent microbicides we will carry out an analysis of anti-HIV peptides derived from gp340 using QSAR modeling. These studies will be based on data obtained from the above formulation studies, stability data, anti-bacterial and spermicidal data. The overall agenda of a QSAR study is to analyze structure-function relationships that may guide the design of more suitable synthetic drugs.

The measured physicochemical properties (e.g. charge, hydrophobicity, secondary structure, HPLC retention times, micelle affinity, etc.) and bioactivity (e.g. binding to gp120, inhibition of HIV infectivity, anti-bacterial activity, cytotoxicity, etc.) of analogs and covalent modifications of these sequences will be determined and entered into a database designed for multivariate analysis and modeling (SIMCA-P11, Umetrics, Umea, Sweden, Spotfire DecisionSite Statistics, Spotfire, U.S., Somerville, Mass.). In addition to the measured properties of the peptides, a battery of calculated parameters including: net charge at pH 7, Eisenberg, Garnier and Chou-Fasman helix propensities, Kyte-Doolittle hydrophobicity; Emini surface index, mean hydrophobic moment and mean charge moment will be determined using the following software (Acclelrys GCG, San Diego, Calif.; EMBOSS, etc.) as recommended by Helberg and LeJon (Hellberg, S., Sjostrom, M., Skagerberg, B. and Wold, S. (1987). Peptide quantitative structure-activity relationships, a multivariate approach. J Med Chem 30(7): 1126-1135; Lejon, T., Strom, M. B. and Svendsen, J. S. (2001). Antibiotic activity of pentadecapeptides modelled from amino acid descriptors. J Pept Sci 7(2): 74-81). The importance of these calculated properties is that they facilitate predictive modeling of novel structures, since the effects of variations in structure on calculated properties can be determined in silico. In QSAR analysis, the bioactivity of each compound (usually expressed as the log) is correlated with a QSAR which consists of the sum of terms derived from the measured and calculated properties of the compound weighted according to statistical analysis of the whole database. Thus the collective effects of all the compounds in the data set contribute to the calculated QSAR value. For example, a strong correlation between hydrophobicity and bioactivity in the database would result in a higher weighting for this property in each QSAR value. Spotfire uses a pattern recognition approach, clustering the data into distinct groups, and presenting it in graphical form for interactive interrogation. The SIMCA methodology reduces the total number of variables in the QSAR by applying principle component and projection analysis to the database. This results in the generation a few derived parameters which largely represent the overall behavior of the data set. The database will be updated and reanalyzed continuously as new compounds are tested to provide mid-course corrections to the synthetic program. The results will be used to iteratively guide the synthesis of additional peptide derivatives and mimetics. The extent to which additional agents will be synthesized depends on the overall performance of the structures generated in the R21, when formulation, stability and anti-bacterial data become available.

### Example 21

### The Effects of SRCR Mimetics to Enhance the Bioactivity of a Series of Other Microbicides

Combination Microbicides

We will perform a set of in vitro studies to evaluate the possibility of combining our SRCR mimetics with other selected anti-HIV agents. At this point the most likely candidates would be Cyanovirin-N, 12p1 peptide, UC781 or TMC120, and Pro 2000. We would first obtain these other microbicidal agents and evaluate their anti-HIV activity as compared to the agents identified as gp340 peptide analogs, derivatives or fragments thereof as identified through the procedures disclosed herein. These assays would monitor HIV infectivity in vitro, using techniques described previously. The protocol would involve testing each agent alone and in combination with one of our newly developed mimetics to determine if there is any value to a combination microbicide (i.e., synergistic vs. additive effect). For these studies the concentration of one drug is kept at a dosage giving 50% inhibition, and then the second agent is titered alone or in combination with the first drug. For data analysis we will use the 3D modeling program for determining synergy (MacSynergyll), first described by Prichard and Shipman (Prichard, M. N. and Shipman, C., Jr. (1990). A three-dimensional model to analyze drug-drug interactions. Antiviral Res 14(4-5): 181-205). Synergy volumes of >50 μM²% will be considered synergistic, while lower values will be assumed to have additive effects. If synergy is observed with any of the drugs, we will further examine those combinations using biosensor technology in order to understand the mechanism behind the bioactivity. For biosensor experiments with compounds that interact with gp120 (eg., 12p1 and SRCR mimetics) we will determine whether the binding of the first drug increases the Ka or Kd for binding of the second drug. These types of studies will help illuminate the mechanism of action, and more importantly help in designing modified HIV inhibitors.

### Example 22

### In Vivo Toxicity Studies with Non-GMP Material

Evaluation of Safety in Animals and Humans.

The small anti-HIV molecules identified through the procedures described herein will be evaluated in a standard Rabbit Vaginal Irritation Study, carried out at a contract site such as SRI International. Typically groups of 5 female rabbits including drugs at 3 dose levels and a positive (N-9) and negative control (placebo) will be utilized. Drugs are administered daily for 10 days and evaluations include (a) clinical observation, (b) gross necroscopy, (c) histological evaluation, and (d) calculation of the mean irritation score. If results of this study demonstrate little or no irritation, and little penetration into tissue, we will proceed to transfer the manufacture of materials to a GMP facility and prepare material for future human clinical trials. Non-GMP material will be used for additional toxicology studies including genotoxicity, pharmacokinetics after intravaginal application of drug and in vitro hepatic microsome studies. These studies would be contracted out to the appropriate testing facilities. Our role would be to evaluate the same samples in our laboratories for drug purity, stability, and bioactivity.

As soon as GMP material becomes available our goal is to initiate clinical phase I trials to assure safety. We will also carry out ex vivo bioactivity assays by obtaining CVL samples from women and evaluating these for bioactivity (anti-HIV testing to determine levels of inhibition in comparison to neat drug, and formulated drug as used in the trial). These studies would be modeled after those described by Herold and coworkers (Keller, M. J., Zerhouni-Layachi, B., Cheshenko, N., John, M., Hogarty, K., Kasowitz, A., Goldberg, C. L., Wallenstein, S., Profy, A. T., Klotman, M. E. and Herold, B. C. (2006). PRO2000 Gel Inhibits HIV and Herpes Simplex Virus Infection Following Vaginal Application: A Double-Blind Placebo-Controlled Trial. J Infect Dis 193(1): 27-35) using Pro2000 as the microbicide. This would permit us to determine if anti-HIV activity is maintained in vitro after in vivo exposure.

What is claimed is:

1. A polyvalent complex of a biocompatible polymer, said biocompatible polymer selected from polymaleic anhydride and poly(ethylene/maleic anhydride) copolymers, said biocompatible polymer having a molecular weight in the range of about 1,000 to about 1,000,000; and a plurality of monomeric peptides covalently bound thereto, said peptides selected from antimicrobial peptides, antiviral peptides, and antifungal peptides; wherein said polyvalent complex exhibits activity that ranges up to on the order of 10 times the activity demonstrated by the peptides alone.

2. The complex of claim 1, wherein the peptide is selected from the group consisting of Antiviral protein Y3, Alloferon 1, Lactoferricin B, hexapeptide, Tricyclic peptide RP, indolicidin, GNCP-1, GNCP-2, HNP-1 Defensin, HNP-2 Defensin, HNP-3 Defensin, CORTICOSTATIN III (MCP-1), CORTICOSTATIN IV (MCP-2), NP-3A defensin, Protegrin 2, Protegrin 3, Protegrin 4, Protegrin 5, RatNP-1, RatNP-2, RatNP-3, RatNP-4, Caerin 1.1, Circulin A (CIRA), Circulin B (CIRB), Cyclopsychotride A (CPT), Ginkobilobin, and Alpha-basrubrin.

3. The complex of claim 1, wherein the peptide is selected from an antimicrobial peptide containing at least one combination of arginine (R) and tryptophan (W), as a single unit or multiple units and in any orientation.

4. The complex of claim 1, wherein the peptide is selected from a fragment of salivary agglutinin protein (gp340) that binds to the gp120 envelope of a retrovirus selected from the group consisting of SEQ ID NOS: 1, 2, 3, 4, 7, and any combination thereof.

5. The complex of claim 2, wherein the peptide is Indolicidin.

6. The complex of claim 1, wherein the polymer is ethylene/maleic anhydride copolymer.

7. The complex of claim 1, which is antimicrobially active water-soluble reaction product of ethylene/maleic anhydride copolymer and indolicidin.

8. A pharmaceutical composition comprising a pharmaceutically acceptable carrier and a pharmaceutically effective amount of the complex of claim 1.

9. The pharmaceutical composition of claim 8, wherein the carrier is a parenteral carrier, oral or topical carrier.

10. A method for preventing, treating, ameliorating or managing a disease or condition which comprises administering to a patient in need of such prevention, treatment, amelioration or management, a prophylactically or therapeutically effective amount of the pharmaceutical composition of claim 8.

11. The method of claim 10, wherein the disease or condition is or results from a bacterial infection.

12. The method of claim 10, wherein the disease or condition is or results from a viral infection.

13. The method of claim 10, wherein the disease or condition is or results from a fungal infection.

14. A method for preventing, treating, ameliorating or managing a disease or condition, which comprises administering to a patient in need of such prevention, treatment, amelioration or management a prophylactically or therapeutically acceptable amount of a complex of claim 1, or the pharmaceutical composition of claim 8, wherein the disease or condition results from or is caused by bacterial infection, viral infection or fungal infection.

15. A method of inhibiting the infectivity of a retrovirus, the method comprising;
a) contacting a retrovirus virion with a substantially purified preparation of a soluble fragment of gp340, or an analog, or a derivative, or a variant or a mimic thereof; and
b) incubating the virion with the soluble fragment of gp340, or analog, or derivative, or variant, or mimic thereof, for a period of time sufficient to inhibit the infectivity of the retrovirus.

16. The method of claim 15, wherein the soluble fragment consists essentially of a segment of gp340 that binds to the gp120 envelope glycoprotein of a retrovirus, and wherein the fragment consists essentially of an N-terminal sequence of gp340, as set forth in SEQ ID NO: 1, a consensus scavenger receptor cysteine rich (SRCR) sequence consisting essentially of an amino acid sequence as set forth in SEQ ID NO: 4, a polypeptide having 16 amino acid residues as set forth in SEQ ID NOs: 3 or 7 or a polypeptide having 11 amino acid residues as set forth in SEQ ID NO: 2.

17. A method of inhibiting or preventing binding or spread of a retrovirus to a host cell, comprising contacting a retrovirus virion with a composition comprising a soluble gp340 fragment, or analog, or mimic thereof for a period of time sufficient to inhibit the spread of the retrovirus to the host cell.

18. The method of claim 17, wherein the fragment consists essentially of a segment of gp340 that binds to the gp120 envelope glycoprotein of a retrovirus, and wherein the fragment consists essentially of an N-terminal sequence of gp340, as set forth in SEQ ID NO: 1 or a consensus scavenger receptor cysteine rich (SRCR) sequence consisting essentially of an amino acid sequence as set forth in SEQ ID NO: 4, a polypeptide having 16 amino acid residues as set forth in SEQ ID NOs: 3 or 7 or a polypeptide having 11 amino acid residues as set forth in SEQ ID NO: 2.

19. A method of inhibiting infectivity of a retrovirus, comprising administering to a subject a pharmaceutical composition comprising a therapeutically effective amount of a soluble gp340 fragment, or analog, or derivative, or variant, or mimic thereof, and a pharmaceutically acceptable carrier.

20. The method of claim 19, wherein the fragment consists essentially of an N-terminal sequence of gp340, as set forth in SEQ ID NO: 1 or a consensus scavenger receptor cysteine rich (SRCR) sequence consisting essentially of an amino acid sequence as set forth in SEQ ID NO: 4, a polypeptide having 16 amino acid residues as set forth in SEQ ID NOs: 3 or 7 or a polypeptide having 11 amino acid residues as set forth in SEQ ID NO:2, or any sequence derived by combinatorial searches that competes effectively for binding to gp120 of the HIV envelope protein with any one of SEQ ID NOS: 1, 2, 3, 4 or 7.

21. The method of either one of claims 19 or 20, wherein the composition is administered to a mucosal surface.

22. The method of claim 21, wherein the mucosal surface is selected from the group consisting of the oral mucosa, the rectal mucosa, and the vaginal mucosa.

23. The method of any one of claims 15-20, wherein the soluble gp340 fragment, analog or mimic thereof is administered prophylactically or therapeutically.

24. The method of any one of claims 15-20, wherein the retrovirus is a human immunodeficiency virus.

25. The method of claim 24, wherein the human immunodeficiency virus is HIV-1.

26. The method of any one of claims 16, 18, or 20, wherein the sequence of any one of SEQ ID NOS: 1, 2, 3, 4 or 7 comprises one or more conservative amino acid substitutions.

27. The method of any one of claims 16, 18, or 20, wherein the sequence of any one of SEQ ID NOS: 1, 2, 3, 4 or 7 or a competitive sequence selected combinatorially that exhibits virucidal activity.

28. The method of any one of claims 15-20, wherein the soluble gp340 fragment, or analog, or derivative, or variant, or mimic thereof, is administered topically.

29. The method of any one of claims 15-20, wherein the soluble gp340 fragment, analog, derivative, variant, or mimic thereof is effective against multiple clades of human immunodeficiency virus or against multi-drug resistant strains of human immunodeficiency virus.

30. The method of any one of claims 16, 18, or 20, wherein the fragment, analog, derivative, variant, or mimic thereof is stabilized by covalent modification of the peptide structure to incorporate one or more disulfide or olefinic H-bonds into the peptide structure.

31. The method of any one of claims 16, 18, or 20, wherein the soluble gp340 fragment, or analog, or derivative, or variant, or mimic thereof is stabilized by attachment to a biocompatible polymeric scaffold or presented in a polyvalent dendrimeric complex.

32. The method of claim 31, wherein the biocompatible polyineric scaffold comprises a polymaleic anhydride and poly(ethylene/maleic anhydride) copolymer, and wherein said copolymer has a molecular weight in the range of about 1,000 to about 1,000,000.

33. The method of claim 31, wherein the polyvalent dendrimeric complex comprises a biocompatible polymer selected from the group consisting of three or more lysine residues and an optional spacer molecule, polyamidoamine, and a plurality of monomeric peptides covalently bound thereto, and wherein said peptides are selected from the group consisting of a soluble gp340 fragment, an analog, or derivative, or variant, or mimic thereof as set forth in any one of SEQ ID NOS: 1, 2, 3, 4 or 7 and alternative sequences that compete with the natural SAG protein sequences.

34. The method of claim 30, wherein the covalent modification results in stabilization of the peptide structure without the need for introducing side claims that alter the affinity of the peptide for the target.

35. A conformationally restricted protein-secondary structure mimetic of a soluble gp340 fragment having anti-HIV activity, wherein the mimetic has significantly higher antiviral activity and lower toxicity than the activity of the parental anti-HIV peptide, wherein the parental anti-HIV peptide consists essentially of the sequence of any one of SEQ ID NOS: 1, 2, 3, 4 or 7 and wherein said mimetic is identified on the basis of its ability to bind to the stem of the V3 loop of the HIV envelope protein and prevent the infectivity or spread of the HIV virus.

36. A pharmaceutical composition comprising a peptide or peptide fragment of a soluble gp340 protein, or an analog, or a derivative, or a variant, or a mimic thereof, and a pharmaceutically acceptable carrier, wherein the peptide or peptide fragment, analog, derivative, variant, or mimic thereof consists essentially of the gp120 binding domain of gp340, wherein the peptide or peptide fragment, analog, derivative, variant or mimic thereof inhibits infectivity of a retrovirus, and wherein the gp120 binding domain of gp340 consists essentially of an N-terminal sequence of gp340 as set forth in SEQ ID NO: 1 or a peptide of about 11 to 16 amino acids in length as set forth in the amino acid sequence of either one of SEQ ID NOS: 2, 3, 4 or 7.

37. The pharmaceutical composition of claim 36, formulated for topical delivery.

38. The pharmaceutical composition of claim 36, formulated for delivery to a mucosal surface.

39. The pharmaceutical composition of claim 38, wherein the mucosal surface is selected from the group consisting of the oral mucosa, the rectal mucosa, and the vaginal mucosa.

40. The pharmaceutical composition of claim 36, wherein the retrovirus is a human immunodeficiency virus.

41. The pharmaceutical composition of claim 40, wherein the human immunodeficiency virus is HIV-1.

42. The pharmaceutical composition of claim 36, wherein the sequence of any one of SEQ ID NOS: 1, 2, 3, 4 or 7 contains one or more conservative amino acid substitutions.

43. The pharmaceutical composition of claim 36, wherein the soluble gp340 fragment, analog, derivative, variant, or mimic thereof exhibits virucidal activity.

44. The pharmaceutical composition of claim 36, wherein the soluble gp340 fragment, analog, derivative, variant, or mimic thereof is effective against multiple clades of human immunodeficiency virus or against multi-drug resistant strains of human immunodeficiency virus.

45. The pharmaceutical composition of claim 36, wherein the soluble gp340 fragment, analog, derivative, variant, or mimic thereof is stabilized by covalent modification of the peptide structure to incorporate one or more disulfide or olefinic H-bonds into the peptide structure.

46. The pharmaceutical composition of claim 36, wherein the soluble gp340 fragment, analog, derivative, variant, or mimic thereof is attached to a biocompatible polymeric scaffold or presented in a polyvalent dendrimeric complex.

47. The pharmaceutical composition of claim 46, wherein the biocompatible polymeric scaffold comprises a polymaleic anhydride and poly(ethylene/maleic anhydride) copolymer, and wherein said copolymer has a molecular weight in the range of about 1,000 to about 1,000,000.

48. The pharmaceutical composition of claim 46, wherein the biocompatible polymeric scaffold comprises a polymer selected from the group consisting of:
Poly(2-hydroxyethyl methacrylate) [12% soln. in ethanol], Poly(4-vinylphenol) [MW 22,000] Poly(4-vinylphenol) [MW 9,000-11,000], Poly(acrylonitrile/butadiene/styrene) powder Poly(acryloyl chloride), 25% soln. in dioxane, Poly(butadiene/maleic anhydride) 1:1 (molar), 25% soln. in acetone, Poly(ethylene glycol) monomethyl ether, Poly(ethylene/maleic anhydride) 1:1 (molar), Poly(maleic anhydride 1-octadecene) 1:1 (molar), Poly(maleic anhydride), Poly(methacryloyl chloride), 25% soln. in dioxane, Poly(propylene oxide), cyclocarbonate terminated, Poly(styrene/maleic anhydride) [67:33], Poly(styrene/maleic anhydride) [75:25], Poly(styrene/maleic anhydride) [9011-13-6], Poly(vinyl methyl ketone), Poly(vinylferrocene) and Polyacrolein.

49. The pharmaceutical composition of claim 46, wherein the polyvalent dendrimeric complex comprises a biocompatible polymer selected from the group consisting of three or more lysine residues and an optional spacer molecule, polyamidoamine, and a plurality of monomeric peptides covalently bound thereto, wherein said peptides are selected from the group consisting of a soluble gp340 fragment, analog or mimic thereof as set forth in SEQ ID NOS: 1, 2, 3, 4 or 7

50. The pharmaceutical composition of claim 36, comprising a conformationally restricted protein-secondary structure mimetic having anti-HIV activity, wherein said mimetic exhibits anti-HIV activity that ranges about 10 times the anti-viral activity of the parental anti-HIV peptide and lower cytotoxicity, wherein said parental anti-HIV peptide consists essentially of the sequence of any one of SEQ ID NOS: 1, 2, 3, 4, or 7 and wherein said mimetic is identified on the basis of its ability to bind to the stem of the V3 loop of the HIV envelope protein and prevent the infectivity or spread of the HIV virus.

51. A pharmaceutical composition comprising the mimetic of claim 35 and a pharmaceutically acceptable carrier.

52. The pharmaceutical composition of claim 50, formulated for oral delivery, parenteral delivery or topical delivery.

53. The pharmaceutical composition of claim 51 useful for prophylactic or therapeutic treatment.

54. A polyvalent complex of a biocompatible polymer, said biocompatible polymer selected from a polymaleic anhydride and poly (ethylene/maleic anhydride) copolymer, said biocompatible polymer having a molecular weight in the range of about 1,000 to about 1,000,000; and a plurality of monomeric peptides that inhibit HIV infectivity covalently bound thereto, wherein said peptides are selected from the group consisting of SEQ ID NOS: 1, 2, 3, 4, or 7 or fragments, analogs, derivatives, variants or mimics thereof, and wherein the polyvalent complex exhibits activity that ranges up to about 10 times the activity demonstrated by the peptides alone.

55. A pharmaceutical composition comprising the polyvalent complex of claim 53 and a pharmaceutically acceptable carrier.

56. The pharmaceutical composition of claim 54, wherein the composition is formulated for oral delivery, parenteral delivery or topical delivery.

57. A polyvalent dendrimeric complex, said complex comprising a biocompatible polymer selected from the group consisting of three or more lysine residues and an optional spacer molecule, and a plurality of monomeric peptides covalently bound thereto, and wherein said peptides are selected from the group consisting of soluble gp340 fragments, analogs, derivatives, variants or mimics thereof as set forth in SEQ ID NOS: 1, 2, 3, 4 or 7

58. A polyvalent dendrimeric complex, said complex comprising a dendrimer selected from the group consisting of a polyamidoamine (PAMAM) dendrimer, a PAMAM (EDA) dendrimer, a poly(Propyleneimine) (PPI) dendrimer and a polylysine dendrimer, and a plurality of monomeric peptides covalently bound thereto, and wherein said peptides are selected from the group consisting of soluble gp340 fragments, analogs, derivatives, variants or mimics thereof as set forth in SEQ ID NOS: 1, 2, 3, 4 or 7

59. A pharmaceutical composition comprising the polyvalent dendrimeric complex of claim 54 and a pharmaceutically acceptable carrier.

60. The pharmaceutical composition of claim 57, formulated for delivery via the oral route, the parenteral route or the topical route.

61. The composition of claim 57, for prophylactic or therapeutic use.

62. A method of inhibiting HIV-1 infectivity comprising contacting the HIV-1 virion with a pharmaceutical composition of either one of claims 36 or 46 and incubating for a time period sufficient to prevent or inhibit HIV-1 infectivity.

63. The method of claim 60, wherein the prevention or inhibition of HIV-1 infectivity is achieved by the peptides in the composition binding to gp120 of the HIV-1 envelope.

64. The method of claim 61, wherein the binding of the gp120 envelope of HIV-1 occurs in the V3 loop of the envelope protein.

65. A method of inhibiting the HIV-1 infectivity of a biological fluid comprising treating said fluid with the composition of either one of claims 36 or 46.

66. The method of claim 65, wherein the biological fluid is selected from the group consisting of cervical vaginal lavage, semen, tears, saliva, bronchial alveolar fluid, blood, serum, plasma and infective oral secretions.


SEQUENCE LISTING (8 sequences)

SEQ ID NO: 1 - protein, 269 aa, Artificial Sequence
  synthetic
MGISTVILEMCLLWGQVLSTRTTDYASLIPSEVPLDPTVAEGSPFPSESTLESTVAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISRELVDPNSQISARLQVDGTGSKLAAAQLWTRASQPELAPEDPEDLEHHHHHHHH

SEQ ID NO: 2 - protein, 11 aa, Artificial Sequence
  synthetic
GRVEVLYRGSW

SEQ ID NO: 3 - protein, 16 aa, Artificial Sequence
  synthetic
QGRVEVLYRGSWGTVC

SEQ ID NO: 4 - protein, 109 aa, Artificial Sequence
  synthetic
GSESSLALRLVNGGDRCQGRVEVLYRGSWGTVCDDSWDTNDANVVCRQLGCGWAMSAPGNARFGQGSGPIVLDDVRCSGHESYLWSCPHNGWLSHNCGHHEDAGVICSA

SEQ ID NO: 5 - protein, 1785 aa, Homo sapiens [sequence omitted]

SEQ ID NO: 6 - protein, 15 aa, Artificial Sequence
  synthetic
CTRPNYNKRKRIHIG

SEQ ID NO: 7 - protein, 16 aa, Artificial Sequence
  synthetic
QGRVEVLYRGSWGEVC

SEQ ID NO: 8 - protein, 15 aa, Artificial Sequence
  synthetic
VQINCTRPNYNKRKR